(19) United States
(12) Patent Application Publication
(10) Pub. No.: US 2026/0275370 A1
(43) Pub. Date: Sep. 17, 2026

(54) PLANTS HAVING INCREASED YIELD, BIOMASS, AND STRESS TOLERANCE

(71) Applicant: Aferna, Inc., Saint Louis, MO (US)

(72) Inventors: Ye Fu, Belmont, MA (US); Jon Schmuke, Saint Louis, MO (US)

(21) Appl. No.: 19/472,276

(22) PCT Filed: Apr. 3, 2024
(86) PCT No.: PCT/US2024/022871

§ 371 Date: Oct. 3, 2025

Related U.S. Application Data
(60) Provisional application No. 63/494,448, filed on Apr. 5, 2023.

(51) Int. Cl. C12N 15/82; C12N 9/02

(52) U.S. Cl. CPC C12N 15/8273; C12N 9/0071

(57) ABSTRACT

The present invention is directed towards a transgenic plant that has an increased biomass, yield and/or abiotic stress tolerance. In particular, the present invention is directed to a transgenic plant or plant cell in which a nucleic acid molecule encoding an RNA demethylase FTO (fat mass and obesity-associated) protein is introduced. Also provided by the disclosure are methods of producing the plants with increased biomass, yield and/or abiotic stress tolerance.

### CROSS-REFERENCE TO RELATED APPLICATIONS

This application claims priority to U.S. Provisional Application No. 63/494,448, filed Apr. 5, 2023, the entire contents of which is incorporated by reference herein.

### TECHNICAL FIELD

The present invention is directed to plants exogenously expressing the polynucleotide(s), the nucleic acid construct(s), polypeptide(s) of the invention and methods of producing the same. In particular, the present invention is directed to introducing polynucleotide(s), the nucleic acid construct(s), and/or polypeptide(s) of the invention into plant(s) to increase the biomass and/or yield, and/or drought tolerance of the plant(s).

### INCORPORATION BY REFERENCE OF SEQUENCE LISTING

The present application is being filed along with a Sequence Listing in electronic format. The Sequence Listing is provided as a file entitled INGRN.005WO_ST26.xml, created on Apr. 2, 2024 which is 108,117 bytes in size. The information in the electronic Sequence Listing is incorporated herein by reference in its entirety.

### BACKGROUND

Changing climate conditions, increasing population, and food scarcity present a formidable challenge in agriculture. Conventional methods for improving crops and horticultural plants have often utilized selective breeding technologies to identify plants with desired properties. This kind of selective breeding technology has several disadvantages in being labor intensive and resulting in plants with heterogeneous genetic complements.

In agriculture, genetic engineering has led to crops with a variety of advantageous enhancements including yield, biomass, pest resistance, herbicide resistance, vigor, and stress tolerance. Transgenic varieties in commercial use have decreased the use of chemical pesticides by 37%, increased the yield of crops by 22%, and increased the profit of farmers by 68%. Most current transgenic technologies, however, increase the yield indirectly through their anti-insect and anti-herbicide properties, etc. Due to increasing needs and challenges, there remains a strong need for development of plants that can withstand their ever-changing environment while increasing yield and biomass.

### SUMMARY OF THE INVENTION

In some aspects, the techniques described herein relate to a plant in which a nucleic acid molecule encoding a fat mass and obesity-associated factor (FTO) is introduced, wherein the FTO has at least the following domains—nuclear localization signal, promoter, coding sequence, and terminating sequence.

In some aspects, a plant in which a nucleic acid molecule encoding an FTO is introduced, wherein the FTO has at least the following domains—a promoter, a nuclear localization signal, a coding sequence, and a terminator.

In other aspects, the FTO nucleic acid molecule is derived from vertebrates, invertebrates, algae, or oomycetes.

In some aspects the plant contains a nucleic acid molecule encoding an FTO that has at least 80% identity to any one of SEQ ID NOs: 34-38.

The plant exhibits at least a twofold increase in biomass relative to a plant not containing the nucleic acid molecule in some aspects. In other aspects the plant exhibits at least a twofold increase in yield relative to a plant not containing the nucleic acid molecule.

The plant, in some aspects is selected from the group consisting of Arabadopsis, canola. wheat, corn, soy, millet, sorghum, cotton, tomato, grapes, peanuts, rice, lettuce, melon, sweet potato, carrot, cucumber, sugar cane, alfalfa, Camelina or PennyCress.

In some aspects a plant, plant cell, tissue, or seed, each having in their genome a nucleic acid molecule comprising a nucleotide sequence of SEQ ID No: 34-38. In other aspects, the plant, plant cell, tissue, or seed has a nucleotide sequence having at least 80% homology to any one of SEQ ID NOs. 34-38.

The nucleic acid molecule further has a promoter, a nuclear localization signal, a coding sequence, and a termination sequence and is expressed in the nucleus in some aspects

The plant expressing the nucleic acid sequence exhibits at least a twofold increase in seed yield, biomass, or yield in some aspects.

The plant expressing the nucleic acid sequence is selected from the group consisting of canola, wheat, soy, millet, sorghum, cotton, tomato, grapes, peanuts, rice, lettuce, melon, sweet potato, carrot, cucumber, sugar cane, alfalfa, Camelina, or PennyCress.

In other aspects, a plant expressing the amino acid sequence having at least a 90% homology to any one of SEQ ID NOs 6-30, and 39-71. The plant further expressing the amino acid sequence in the nucleus in some aspects.

The plant expressing the amino acid sequence exhibits at least a twofold increase in seed yield, biomass, or yield in some aspects and may be selected from the group consisting of canola, wheat, soy, millet, sorghum, cotton, tomato, grapes, peanuts, rice, lettuce. melon, sweet potato, carrot, cucumber, sugar cane, alfalfa, Camelina, or PennyCress.

In some embodiments, a plant comprising plant cells comprising a nucleic acid molecule encoding a fat mass and obesity-associated factor (FTO) gene is provided. In some embodiments, the expression of the said nucleic acid molecule results in one or more of increased yield, biomass, growth rate, drought tolerance and abiotic stress tolerance relative to a plant of same variety lacking the nucleic acid molecule encoding the FTO gene. In some embodiments, the FTO nucleic acid molecule is from vertebrates, invertebrates, algae, or oomycetes. In some embodiments, the nucleic acid molecule further comprises a promoter, a nuclear localization signal, a coding sequence, and a termination sequence.

In some embodiments, the nucleic acid molecule encoding an FTO has at least 80% identity to any one of SEQ ID NOs: 34-38.

In some embodiments, the plant exhibits at least a twofold increase in biomass relative to the plant not containing the nucleic acid molecule. In some embodiments, the plant exhibits at least a twofold increase in yield relative to the plant not containing the nucleic acid molecule. In some embodiments, the plant exhibits an increase in drought tolerance relative to the plant not containing the nucleic acid molecule.

In some embodiments, the plant is selected from the group consisting of Arabadopsis, canola, wheat, corn, soy, millet, sorghum, cotton, tomato, grapes, peanuts, rice, lettuce, melon, sweet potato, carrot, cucumber, sugar cane, alfalfa, Camelina or PennyCress.

In some embodiments, the FTO gene is expressed in a nucleus of said plant cells.

In some embodiments, the FTO gene encodes a polypeptide comprising any one of SEQ ID NOs 6-30 or 39-71 or a polypeptide comprising at least 90% identity therewith.

In some embodiments, a method of generating a modified plant comprising increased yield, growth rate, biomass, vigor, oil content, seed yield, fiber yield, fiber quality, fiber length, photosynthetic capacity, nitrogen use efficiency, drought tolerance and/or abiotic stress tolerance as compared to a wild type plant of the same variety is provided. In some embodiments, the method comprises: (a) genetically modifying a plant cell with an exogenous polynucleotide encoding a polypeptide comprising an amino acid sequence with at least 80% identity to the sequence selected from the group comprising any one of SEQ ID NOs: 6-30, and 39-71, and (b) regenerating the modified plant from said plant cell modified with an exogenous polynucleotide. In some embodiments, the method further comprises producing modified plants from the plant produced in step (b). In some embodiments, the polypeptide is from vertebrates, invertebrates, algae, or oomycetes.

IE some embodiments, the modified plant exhibits at least a twofold increase in biomass relative to a plant not containing the nucleic acid molecule. In some embodiments, the modified plant exhibits at least a twofold increase in yield relative to a plant not containing the nucleic acid molecule. In some embodiments, the modified plant exhibits an increase in drought tolerance relative to a plant not containing the nucleic acid molecule. In sonic embodiments, the plant is selected from the group consisting of Arabadopsis, canola, wheat, corn, soy, millet, sorghum, cotton, tomato, grapes, peanuts, rice, lettuce, melon, sweet potato, carrot, cucumber, sugar cane, alfalfa, Camelina or PennyCress.

### BRIEF DESCRIPTION OF THE DRAWINGS

Some embodiments of the invention are herein described, by way of example only, with reference to the accompanying drawings. The drawings are by way of example and for purposes of illustrative discussion of the embodiments of the invention.

FIG. 1 is a schematic illustration of the pET-28a(+) plasmid containing the T7 promoter used for expressing the polynucleotide sequences of the invention, as in some embodiments of the invention.

FIG. 2 is a schematic illustration of the pET-28a plasmid containing acFTO, SEQ ID No. 9 as in one embodiment of the invention.

FIG. 3 is a picture of a gel illustrating the expression of FTO homologs in E. coli after induction, as in some embodiments of the invention.

FIG. 4 is a schematic representing root length in Seteria plants transformed with hFTO-GFP and mcFTO (SEQ ID No. 7), as in an embodiment of the invention.

FIG. 5 is a schematic illustrating seed weights by event and gene of interest (GOI), as in an embodiment of the invention.

FIG. 6 is a schematic illustrating FTO transgene expression in Seteria expressing SEQ ID Nos. 1, 2, and 7, as in an embodiment of the invention.

FIG. 7 is a schematic illustrating root length in Seteria expressing SEQ ID No. 7, as in one embodiment of the invention.

FIG. 8 is a schematic representing biomass of Seteria transformed with SEQ ID Nos. 9. 6, 8, and 3, as in an embodiment of the invention.

FIG. 9 is a schematic representing seed yield of Seteria transformed with SEQ ID Nos. 9, 6, 8, and 3 as in one embodiment.

FIG. 10 is a schematic representing the expression of SEQ ID NO. 1 in soy, as in one embodiment of the invention.

FIGS. 11A and 111B illustrate images of drought treated soy with an empty vector control (FIG. 11A) and soy transformed with SEQ ID No. 8 (FIG. 11B).

FIGS. 12A, 12B and 12C illustrate images of drought treated soy with an empty vector control (FIG. 12A), soy transformed with SEQ ID. No. 7 (FIG. 12B), and soy transformed with SEQ ID No. 7 (FIG. 12C).

FIG. 13 illustrates expression of hFTO acFTO, niFTO protein detected by Western Blot in Soy plants.

FIG. 14 illustrates detection of hFTO-GFP expression in nuclei in Soy plants.

FIGS. 15A and 15B illustrate the position of the plants used for the drought assay in soy plants transformed with SEQ ID NO:9. FIG. 15A shows a map used to place the correct plant in the right position for the experiment and FIG. 15B shows the well-watered and droughted plants surrounded by the soy border to prevent edge effects.

FIG. 16A illustrates an embodiment of images for score determination of wilting and FIG. 16B illustrates the root length of the well-watered and droughted soy plants transformed with SEQ. ID NO. 9.

FIGS. 17A, 17B and 17C illustrate the drought assay in soy plants transformed with SEQ ID NO:9. FIG. 17A shows a map used to place the correct plant in the right position for the experiment, FIG. 17B shows the wilt score of the well-watered and droughted soy plant leaves and FIG. 17C shows the root length of the well-watered and droughted soy plants transformed with SEQ. ID NO. 9.

FIGS. 18A, 18B, 18C, 18D and 18E illustrate the drought assay in soy plants transformed with SEQ ID NO:7. FIG. 18A shows an overhead image of the plants, FIG. 18B shows the side view of the plants, FIG. 18C shows the root morphology of the well-watered and droughted soy plants transformed with SEQ. ID NO. 7, FIG. 18D shows the root length of the well-watered and droughted soy plants transformed with SEQ. ID NO. 7 and FIG. 18E shows the wilt score of the well-watered and droughted soy plant leaves transformed with SEQ. ID NO. 7.

### DETAILED DESCRIPTION

Previous epigenetic research has generally focused on reversible modifications of DNA and histone. While hundreds of RNA modifications have been discovered, N6-methyladenosine (m6A) has been found to be the most abundant RNA modification in mRNA across all eukaryotes.

mRNAs of all plants contain N6-methyladenoesine (m6A) modification. m6A regulates the metabolic processing of mRNAs, including splicing, nuclear export, stability and protein translation via m6A binding proteins. The modifications are produced by m6A modifying enzymes (currently reported modifying enzyme subunits are classified as MTA (METTL3 homologous gene) and FIP37 (WTAP homologous gene)) and have important regulatory effect on the growth and development of plants. Although the modification has been known for over forty years, its function was not known until demethylase of m6A, FTO protein was characterized (Jia et al, N6-methyladenosine in nuclear RNA is a major substrate of the obesity-associated FTO. Nat Chem Biol, 2011, 7 (12): 885-887), reporting the reversibility of RNA modification for the first time and started the “RNA epigenetics (or Epitranscriptome)” research in 2011.

During the demethylation process of m6A in RNA, FTO produces two relatively stable new modifications, hm6A (N6-hydroxymethyladenosine) and f6A (N6-formyladenosine), which have potential regulatory effects on RNA processing (Fu et al, FTO-mediated formation of N6-hydroxymethyladenosine and N6-fonnyladenosine in mammalian RNA. Nat Commun, 2013, 4:1798). Given the importance of this modification, there are advantages to discovering FTO homologs that produce increased yield and biomass in other plants. However, such efforts are difficult and time consuming as cell localization, transcription efficiency, species specificity and phenotypic effects cannot be predicted owing to variations in the FTO sequences across species.

In some embodiments, homologs of FTO that increase biomass, yield, and stress tolerance in crops disclosed herein is provided. Also provided herein are FTO homolog polypeptides, compositions thereof, and methods of developing plants with increased beneficial traits, including yield, growth rate, biomass, vigor, oil content, seed yield, fiber yield, fiber quality, fiber length, photosynthetic capacity, and/or abiotic stress tolerance.

### Definitions

The illustrative embodiments described in the detailed description, drawings, and claims are not meant to be limiting. Other embodiments may be utilized, and other changes may be made, without departing from the spirit of scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, all technical and scientific terms used herein have the same meaning as commonly understood by one of ordinary skill in the art to which the invention pertains.

The terms “comprises”, “comprising”, “includes” “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

As used herein the phrase “plant yield” refers to the amount (e.g., as determined by weight, volume, processed product, or size) or quantity (numbers) of tissues or organs produced per plant or per growing season. Hence increased yield could affect the economic benefit one can obtain from the plant in a certain growing area and/or growing time.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

The term “isolated” refers to at least partially separated from the natural environment e.g., from a plant cell.

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements. Nucleic acid sequences encoding the polypeptides of the present invention may be optimized for expression. Examples of such sequence modifications include, but are not limited to, an altered G/C content to more closely approach that typically found in the plant species of interest, and the removal of codons atypically found in the plant species commonly referred to as codon optimization.

The phrase “codon optimization” refers to the selection of appropriate DNA nucleotides for use within a structural gene or fragment thereof that approaches codon usage within the plant of interest. Therefore, an optimized gene or nucleic acid sequence refers to a gene in which the nucleotide sequence of a native or naturally occurring gene has been modified in order to utilize statistically-preferred or statistically-favored codons within the plant. The naturally-occurring encoding nucleotide sequence may already, in advance of any modification, contain a number of codons that correspond to a statistically-favored codon in a particular plant species. Therefore, codon optimization of the native nucleotide sequence may comprise determining which codons, within the native nucleotide sequence, are not statistically-favored with regards to a particular plant, and modifying these codons in accordance with a codon usage table of the particular plant to produce a codon optimized derivative. A modified nucleotide sequence may be fully or partially optimized for plant codon usage provided that the protein encoded by the modified nucleotide sequence is produced at a level higher than the protein encoded by the corresponding naturally occurring or native gene. Construction of synthetic genes by altering the codon usage is described in for example PCT Patent Application 93/07278, incorporated by reference herein in its entirety. Thus, the invention encompasses nucleic acid sequences described hereinabove; fragments thereof, sequences hybridizable therewith, sequences homologous thereto, sequences encoding similar polypeptides with different codon usage, altered sequences characterized by mutations, such as deletion, insertion or substitution of one or more nucleotides, either naturally occurring or man induced, either randomly or in a targeted fashion.

As used herein, the term “plant” includes a whole plant and any descendant, cell, tissue, or part of a plant. The term “plant parts” include any part(s) of a plant, including, for example and without limitation: seed (including mature seed and immature seed); a plant cutting; a plant cell; a plant cell culture: a plant organ (e.g., pollen, embryos, flowers, fruits, shoots, leaves, roots, stems, and explants). A plant tissue or plant organ may be a seed, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks.

Plant parts include harvestable parts and parts useful for propagation of progeny plants. Plant parts useful for propagation include, for example and without limitation: seed; fruit: a cutting; a seedling; a tuber; and a rootstock. A harvestable part of a plant may be any useful part of a plant, including, for example and without limitation: flower; pollen: seedling; tuber; leaf; stem; fruit: seed; and root.

A plant cell is the structural and physiological unit of the plant. Plant cells, as used herein, includes protoplasts and protoplasts with a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete producing cell, or a cell or collection of cells that can regenerate into a whole plant. As such, a seed, which comprises multiple plant cells and is capable of regenerating into a whole plant, is considered a “plant part” in embodiments herein.

The plant may be in any form including suspension cultures, embryos, meristematic regions, callus tissue, leaves, gametophytes, sporophytes, pollen, and microspores.

It should be noted that a plant yield can be affected by various parameters including, but not limited to, plant biomass; plant vigor, stress tolerance, growth rate; seed yield; seed or grain quantity; seed or grain quality; oil yield; content of oil, starch and/or protein in harvested organs (e.g., seeds or vegetative parts of the plant); number of flowers (florets) per panicle (expressed as a ratio of number of filled seeds over number of primary panicles); harvest index; number of plants grown per area; number and size of harvested organs per plant and per area; number of plants per growing area (density); number of harvested organs in field; total leaf area; carbon assimilation and carbon partitioning (the distribution/allocation of carbon within the plant): resistance to shade; number of harvestable organs (e.g. seeds), seeds per pod, weight per seed; and modified architecture [such as increase stalk diameter, thickness or improvement of physical properties (e.g. elasticity)].

As used herein the phrase “seed yield” refers to the number or weight of the seeds per plant, seeds per pod. or per growing area or to the weight of a single seed, or to the oil extracted per seed. Hence seed yield can be affected by seed dimensions (e.g., length, width, perimeter, area and/or volume), number of (filled) seeds and seed filling rate and by seed oil content. Hence increase seed yield per plant could affect the economic benefit one can obtain from the plant in a certain growing area and/or growing time; and increase seed yield per growing area could be achieved by increasing seed yield per plant, and/or by increasing number of plants grown on the same given area.

The term “seed” (also referred to as “grain” or “kernel”) as used herein refers to a small embryonic plant enclosed in a covering called the seed coat (usually with some stored food), the product of the ripened ovule of gymnosperm and angiosperm plants which occurs after fertilization and some growth within the mother plant.

The phrase “oil content” as used herein refers to the amount of lipids in a given plant organ, either the seeds (seed oil content) or the vegetative portion of the plant (vegetative oil content) and is typically expressed as percentage of dry weight (10% humidity of seeds) or wet weight (for vegetative portion). It should be noted that oil content is affected by intrinsic oil production of a tissue (e.g., seed, vegetative portion), as well as the mass or size of the oil-producing tissue per plant or per growth period. In one embodiment, increase in oil content of the plant can be achieved by increasing the size/mass of a plant's tissue(s) which comprise oil per growth period. Thus, increased oil content of a plant can be achieved by increasing the yield, growth rate, biomass and vigor of the plant.

As used herein the phrase “plant biomass” refers to the amount (e.g., measured in grams of air-dry tissue) of a tissue produced from the plant in a growing season, which could also determine or affect the plant yield or the yield per growing area. An increase in plant biomass can be in the whole plant or in parts thereof such as aboveground (harvestable) parts, vegetative biomass, roots and seeds.

As used herein the term “root biomass” refers to the total weight of the plant's root(s). Root biomass can be determined directly by weighing the total root material (fresh and/or dry weight) of a plant. Additionally or alternatively, the root biomass can be indirectly determined by measuring root coverage, root density and/or root length of a plant. It should be noted that plants having a larger root coverage exhibit higher fertilizer (e.g., nitrogen) use efficiency and/or higher water use efficiency as compared to plants with a smaller root coverage.

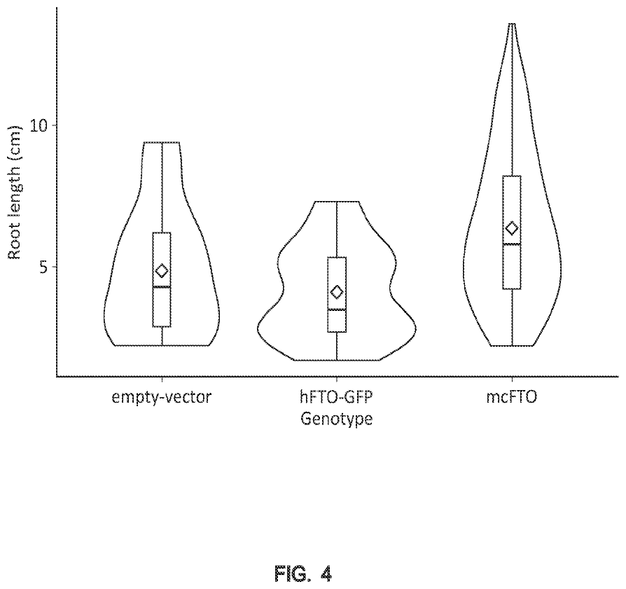

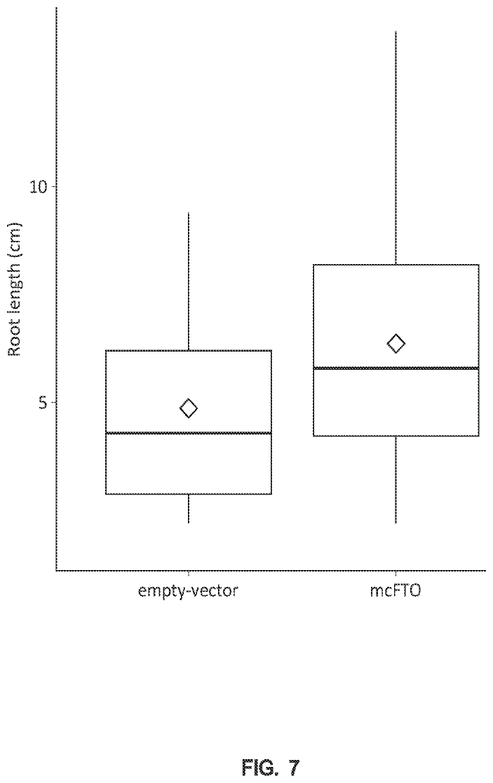

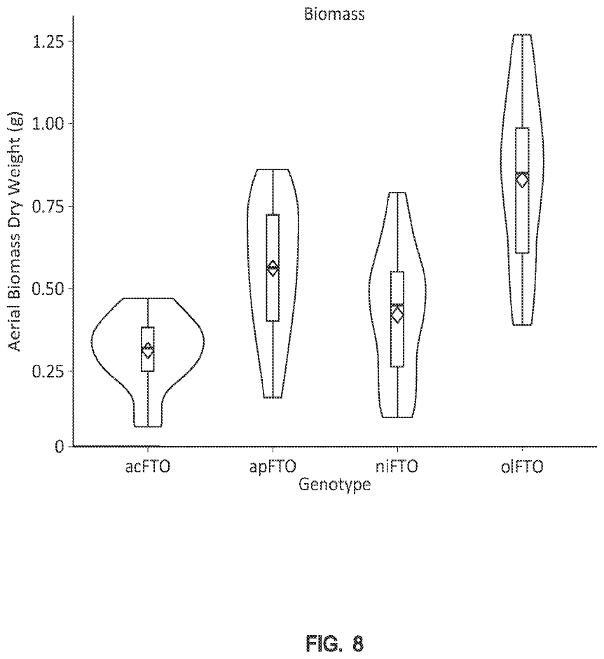

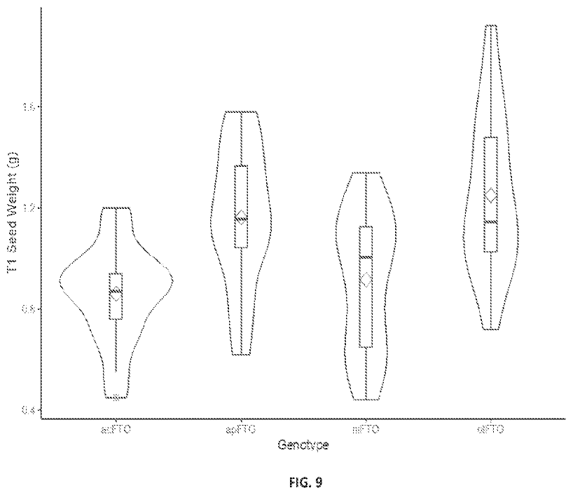

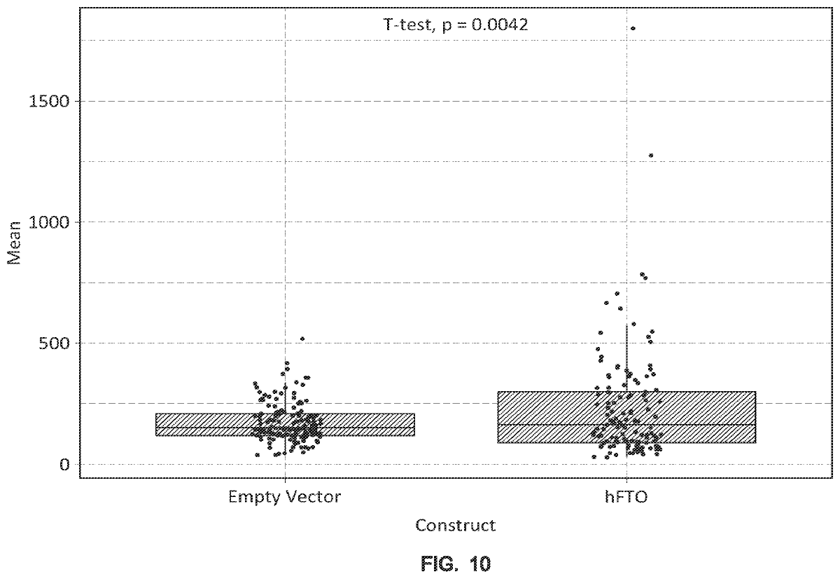

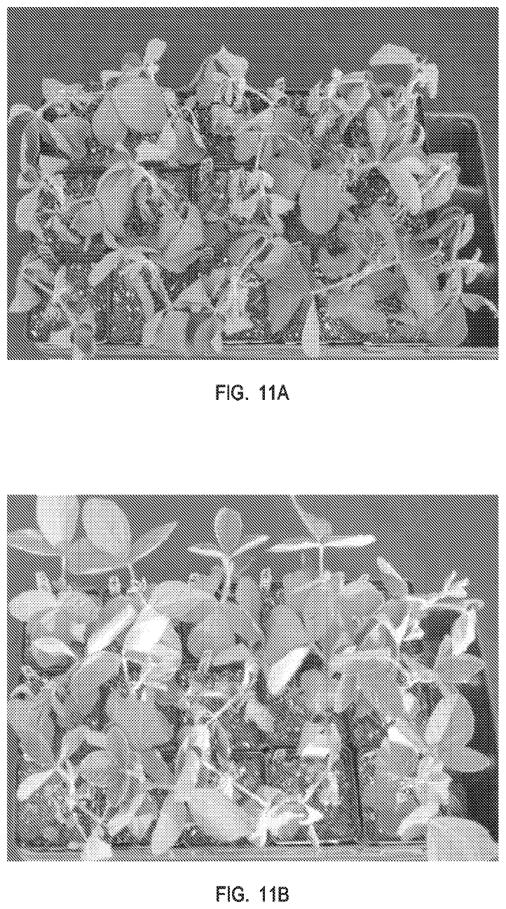

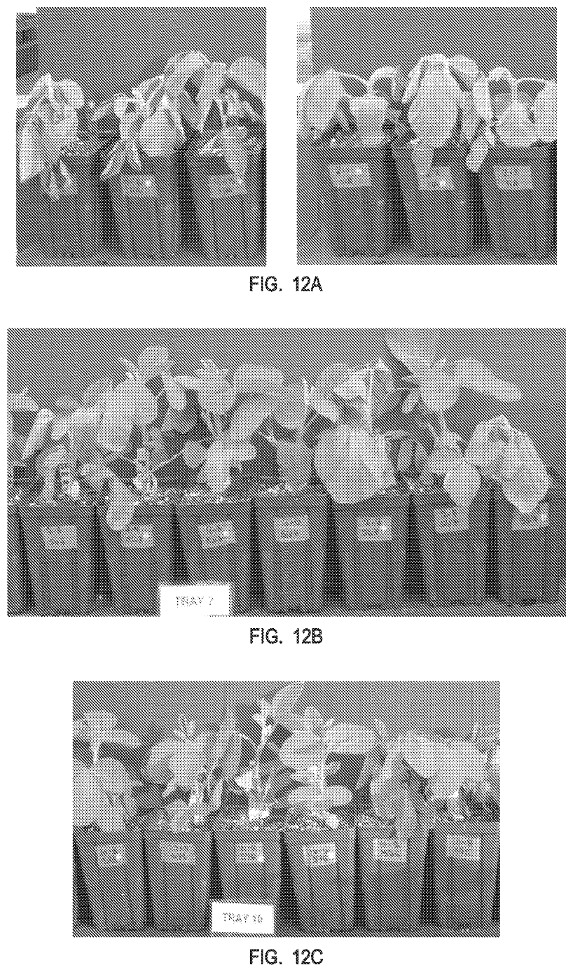

As used herein the phrase “root coverage” refers to the total area or volume of soil or of any plant-growing medium encompassed by the roots of a plant. According to some embodiments of the invention, the root coverage is the minimal convex volume encompassed by the roots of the plant. It should be noted that since each plant has a characteristic root system, e.g., some plants exhibit a shallow root system (e.g., only a few centimeters below ground level), while others have a deep in soil root system (e.g., a few tens of centimeters or a few meters deep in soil below ground level), measuring the root coverage of a plant can be performed in any depth of the soil or of the plant-growing medium, and comparison of root coverage between plants of the same species (e.g., a transgenic plant exogenously expressing the polynucleotide of some embodiments of the invention and a control plant) should be performed by measuring the root coverage in the same depth. According to some embodiments of the invention, the root coverage is the minimal convex area encompassed by the roots of a plant in a specific depth. A non-limiting example of measuring root coverage is shown in FIGS. 4 and 7. As used herein the term “root density” refers to the density of roots in a given area (e.g., area of soil or any plant growing medium). The root density can be determined by counting the root number per a predetermined area at a predetermined depth (in units of root number per area, e.g., mm², cm² or m²).

As used herein the phrase “root length” refers to the total length of the longest root of a single plant.

As used herein the phrase “root length growth rate” refers to the change in total root length per plant per time unit (e.g., per day).

As used herein the phrase “growth rate” refers to the increase in plant organ/tissue size per time (can be measured in cm² per day or cm/day).

As used herein the phrase “photosynthetic capacity” (also known as “Amax”) is a measure of the maximum rate at which leaves are able to fix carbon during photosynthesis. It is typically measured as the amount of carbon dioxide that is fixed per square meter per second, for example as μmol m-2 sec-1. Plants are able to increase their photosynthetic capacity by several modes of action, such as by increasing the total leaves area (e.g., by increase of leaves area, increase in the number of leaves, and increase in plant's vigor, e.g., the ability of the plant to grow new leaves along time course) as well as by increasing the ability of the plant to efficiently execute carbon fixation in the leaves. Hence, the increase in total leaves area can be used as a reliable measurement parameter for photosynthetic capacity increment.

As used herein the phrase “plant vigor” refers to the amount (measured by weight) of tissue produced by the plant in a given time. Hence increased vigor could determine or affect the plant yield or the yield per growing time or growing area. In addition, early vigor (seed and/or seedling) results in improved field stand. Improving early vigor is an important objective of modem rice breeding programs in both temperate and tropical rice cultivars. Long roots are important for proper soil anchorage in water-seeded rice. Where rice is sown directly into flooded fields, and where plants must emerge rapidly through water, longer shoots are associated with vigor. Where drill-seeding is practiced, longer mesocotyls and coleoptiles are important for good seedling emergence. The ability to engineer early vigor into plants would be of great importance in agriculture. For example, poor early vigor has been a limitation to the introduction of maize (Zea mays L.) hybrids based on Corn Belt germplasm in the European Atlantic.

In some embodiments, a plant trait such as yield, growth rate, biomass, vigor, oil content, fiber yield, fiber quality, fiber length, photosynthetic capacity, fertilizer use efficiency (e.g., nitrogen use efficiency) can be determined under stress (e.g., abiotic stress, nitrogen-limiting conditions) and/or non-stress (normal) conditions.

As used herein, the phrase “non-stress conditions” refers to the growth conditions (e.g., water, temperature, light-dark cycles, humidity, salt concentration, fertilizer concentration in soil, nutrient supply such as nitrogen, phosphorous and/or potassium), that do not significantly go beyond the everyday climatic and other abiotic conditions that plants may encounter, and which allow optimal growth, metabolism, reproduction and/or viability of a plant at any stage in its life cycle (e.g., in a crop plant from seed to a mature plant and back to seed again). Persons skilled in the art are aware of normal soil conditions and climatic conditions for a given plant in a given geographic location. It should be noted that while the non-stress conditions may include some mild variations from the optimal conditions (which vary from one type/species of a plant to another), such variations do not cause the plant to cease growing without the capacity to resume growth.

The term “fiber” is usually inclusive of thick-walled conducting cells such as vessels and tracheids and to fibrillar aggregates of many individual fiber cells. Hence, the term “fiber” includes lignin and also refers to (a) thick-walled conducting and non-conducting cells of the xylem: (b) fibers of extraxylary origin, including those from phloem, bark, ground tissue, and epidermis; and (c) fibers from stems, leaves, roots, seeds, and flowers or inflorescences (such as those of Sorghum vulgare used in the manufacture of brushes and brooms). Examples of fiber producing plants, include, but are not limited to, agricultural crops such as cotton, silk cotton tree (Kapok. Ceiba pentandra), desert willow, creosote bush, winterfat, balsa, kenaf, roselle, jute, sisal abaca, flax, corn, sugar cane, hemp, ramie, kapok, coir, bamboo, Spanish moss and Agave spp. (e.g. sisal).

As used herein the phrase “fiber quality” refers to at least one fiber parameter which is agriculturally desired, or required in the fiber industry (further described hereinbelow). Examples of such parameters, include but are not limited to, fiber length, fiber strength, fiber fitness, fiber weight per unit length, maturity ratio and uniformity (further described hereinbelow). In some embodiments, cotton fiber (lint) quality is typically measured according to fiber length, strength and fineness. Accordingly, the lint quality is considered higher when the fiber is longer, stronger and finer.

As used herein the phrase “fiber yield” refers to the amount or quantity of fibers produced from the fiber producing plant.

As disclosed herein, transgenic plants of the present invention can be used for improving myriad of commercially desired traits which are all interrelated as is discussed hereinbelow.

As used herein the term “trait” refers to a characteristic or quality of a plant which may overall (either directly or indirectly) improve the commercial value of the plant.

As used herein the term “increasing” refers to at least about 2%, at least about 3%, at least about 4%, at least about 5%, at least about 10%, at least about 15%, at least about 20%, at least about 30%, at least about 40%, at least about 50%, at least about 60%, at least about 70%, at least about 80%, increase in the trait [e.g., yield. seed yield, biomass, growth rate, vigor, oil content, fiber yield, fiber quality, fiber length, photosynthetic capacity, abiotic stress tolerance, and/or nitrogen use efficiency] of a plant as compared to a native plant or a wild type plant [i.e., a plant not modified with the biomolecules (polynucleotide or polypeptides) of the invention, e.g., a non-transformed plant of the same species which is grown under the same (e.g., identical) growth conditions], or any value in between.

The phrase “expressing within the plant an exogenous polynucleotide” as used herein refers to upregulating the expression level of an exogenous polynucleotide within the plant by introducing the exogenous polynucleotide into a plant cell or plant and expressing by recombinant means, as further described herein below.

As used herein “expressing” refers to expression at the mRNA and optionally polypeptide level.

As used herein, the phrase “exogenous polynucleotide” refers to a heterologous nucleic acid sequence which may not be naturally expressed within the plant (e.g., a nucleic acid sequence from a different species) or which overexpression in the plant is desired. The exogenous polynucleotide may be introduced into the plant in a stable or transient manner, so as to produce a ribonucleic acid (RNA) molecule and/or a polypeptide molecule. It should be noted that the exogenous polynucleotide may comprise a nucleic acid sequence which is identical or partially homologous to an endogenous nucleic acid sequence of the plant.

As used herein, the term “endogenous” as used herein refers to any polynucleotide or polypeptide which is present and/or naturally expressed within a plant or a cell thereof.

As used herein, homologous sequences include both orthologous and paralogous sequences. The term “paralogous” relates to gene-duplications within the genome of a species leading to paralogous genes. The term “orthologous” relates to homologous genes in different organisms due to ancestral relationship. Thus, orthologs are evolutionary counterparts derived from a single ancestral gene in the last common ancestor of given two species (Koonin E V and Galperin M Y (Sequence-Evolution-Function: Computational Approaches in Comparative Genomics. Boston: Kluwer Academic; 2003. Chapter 2, Evolutionary Concept in Genetics and Genomics. Available from: ncbi(dot)nlm(dot)nih(dot)gov/books/NBK20255) and therefore have great likelihood of having the same function. Homology (e.g., percent homology, sequence identity+sequence similarity) can be determined using any homology comparison software computing a painise sequence alignment.

As used herein, “sequence identity” or “identity” in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. [Amino acid substitution matrices from protein blocks. Proc. Natl. Acad. Sci. U.S.A. 1992, 89(22): 10915-9]. Identity (e.g., percent homology) can be determined using any homology comparison software, including for example, the BlastN software of the National Center of Biotechnology Information (NCBI) such as by using default parameters. According to some embodiments of the invention, the identity is a global identity, i.e., an identity over the entire amino acid or nucleic acid sequences of the invention and not over portions thereof.

According to some embodiments of the invention, the term “homology” or “homologous” refers to identity of two or more nucleic acid sequences; or identity of two or more amino acid sequences; or the identity of an amino acid sequence to one or more nucleic acid sequence. According to some embodiments of the invention, the homology is a global homology, i.e., an homology over the entire amino acid or nucleic acid sequences of the invention and not over portions thereof. The degree of homology or identity between two or more sequences can be determined using various known sequence comparison tools.

### FTO Homologs

In some embodiments, the FTO refers to any FTO gene selected from a species comprising Homo sapiens, Acanthaster planci, Patiria miniata, Asterias rubens, Saccoglossus kowalevskii, Patiria miniata, Lytechinus vanegatus, Aphanomyces mvadans, Aphanomyces astaci, Aphanomyces stellatus, Ostreococcus tauri, Micromonas commoda, Aphanomyces cochlioides, Aphanomyces euteiches, Aphanomyces invadans, Ostreococcus lucimarinus CCE9901, Micromonas pusilla CCMP1545, Ostreococcus tauri, Chaetoceros tenuissimus, Pseudo-nitzschia multistriata, Fragilariopsis cylindrus CCMP1102, Thalassiosira pseudonana CCMP1335, Lytechinus variegatus, Nitzschia inconspicua, Guillardia theta CCMP2712, Ectocarpus sp CCAP 1310/34, Ectocarpus siliculosus, Thecamonas trahens ATCC 50062, Polarella glacialis, Aphanomyces invadans, Tribonema minus, Seminavis robusta, Aphanomyces cochlioides, Fragilaria crotonensis, Mayamaea pseudoterrestris, Capsaspora owczarzaki ATCC 30864, Cryptophyta sp CCMP2293, Fragilaria crotonensis, Chaetoceros tenuissimus, synthetic construct, Ectocarpus sp CCAP 1310/34, Aphanomyces astaci, Pseudo-nitzschia multistriata, Ochromonadaceae sp CCMP2298, Ectocarpus siliculosus, Seminavis robusta, Nitzschia inconspicua, Sphaeroforma arctica JP610, Nitzschia inconspicua, Fragilariopsis cylindrus CCMP1102, Fragilaria crotonensis, Thalassiosira oceanica, Polarella glacialis, Lytechinus pictus, Corticium candelabrum, Cylindrotheca closterium, Skeletonema marinoi, Tetraparma gracilis, Triparma retinervis, Triparma columacea, Triparma laevis f inornata, Cylindrotheca closterium, Triparma laevis f longispina, Parmales sp. scaly parma, Triparma columacea, Triparma verrucosa, Triparma strigata, Triparma strigata, Chrysophaeum taylorii, Triparma retinervis, Triparma strigata, Parmales sp. scaly parma, Parmales sp. scaly parma, Alaria esculenta, Alaria esculenta, Cladosiphon okamuranus S strain, Cladosiphon okamuranus S strain, Cyclotella cryptica CCMP332, Macrocystis pyrifera, Macrocystis pyrifera, Minidiscus variabilis CCMP495, Monodopsis C141, Monodopsis C73, Nemacystus decipiens Onna-1, Nemacystus decipiens Onna-1, Nitzschia putrida NJES-4239, Nitzschia putrida NIES-4239, Ochromonas CCMP139, Paraphysomonas imperforata CCMP1604, Phaeocystis antarctica CCMP1374, Prasinoderma coloniale CCMP1413, Proteomonas sulcata CCMP 1175 Saccharina japonica, Tetraselmis striata, Undaria pinnatifida M23, Undaria pinnatifida M23.

In some embodiments, the FTO gene is derived from vertebrates, invertebrates, algae, and/or oomycetes.

In some embodiments, the FTO gene is identified by accession number selected from the group comprising “NP_001073901.1. AAI48443.1, XP_022097537.1. XP_038078604.1, XP_033625687.1, XP_002738337.1, ALR88588.1, XP_038078612.1, XP_038049512.1, XP 041454524.1. XP_041454523.1. XP_008874274.1, RHZ09857.1, RHY54141.1, RHY97650.1, KAF0719040.1, RHY17698.1, RHY56488.1, XP_022840077.1. RQM30574.1, XP_009841386.1, XP_002502764.1. KAG9401836.1. KAF0726784.1, RHY29166.1. XP_001420808.1. XP_003060875.1. OUS43030.1, KAH9102871.1. KAH9187704.1. GFH54458.1, VEU39385.1. OEU13050.1, XP_002287957.1, XP_041454525.1, KAG7347203.1, XP_009841387.1, XP_005838228.1, CAB1105374.1, KAG7364155.1. CBJ31658.1, XP_013753876.1, CAE8644684.1, CAE8611733.1, CAE8644257.1, RHZ18017.1, RHY92689.1, RHY56741.1, RHY00398.1, XP_008874276.1, KAG5193084.1, CAB9501471.1. KAG9401837.1, KAI2507824.1, GKY90516.1, XP_004347827.1, KAJ1495429.1, KA12498707.1, GFH54956.1, AIC63558.1, CAB1117017.1, KAF0752546.1, VEU37468.1, KAJ1427805.1, RL012103.1, CBJ31645.1, CAB9497616.1, RLO00695.1, KAG7357268.1, XP_014148728.1, KAG7360647.1, OEU10178.1, KAI2500024.1, RHY56738.1, RHY00397.1, RHY92688.1, EJK46618.1, CAE8633430.1, XP_054763800.1, XP_054769608.1, XP_062505117.1, XP_062505118.1, CAJ1967500.1, KAK1734719.1, GMI22625.1, GMH73802.1, GMI46646.1, MDA9098936.1, GMH59919.1, CAJ1942268.1, GMH75049.1, GM153731.1, GM145036.1, GMH96566.1, GMH91706.1, GMH91703.1, KAJ8609712.1, GMI10743.1, GMH93314.1, GMI61299.1, esubft_2221_2, esubft_6442, g17329.t1, g9595.t1, CCRYP_016297-RB, estExt_fgenesh2_pg.C_8_t10199, fgenesh2_pm.17_#_163, estExt_Genewisel.C_190154, Monodopsis_C141_contig_8_g54760.t1, Monodopsis_C73_contig_18_g112030.t1, g1621.t1, g11573.t1. NAA08P02010.m1, NAA05P01980.m1, CE122323_2229, fgenesh1_kg.94_#_167_#_TRINITY_DN11056_c0_g1_i3, Phant.0041s0181.1, PRCOL_00005642-RA, fgenesh1_kg.8_#_1316_#_TRINITY_DN4680_c0_g1_i6, SJ15771.1, SJ04115.1, TSEL_005271.t1, TSEL_005270.t1, TSEL_010033.t1, HiC_scaffold 21.g09429.m1, HiC_scaffold_11.g04962.m1.

In some embodiments, the FTO gene comprises an exogenous polynucleotide or a nucleic acid sequence comprising SEQ ID NO: 34, SEQ ID NO: 35, SEQ ID NO: 36. SEQ ID NO: 37 or SEQ ID NO: 38. In some embodiments, the exogenous polynucleotide or the nucleic acid sequence comprises at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 93%, at least about 94%, at least about 95%, at least about 96%, at least about 97%, at least about 98%, at least about 99%, e.g., 100% identical to the nucleic acid sequence selected from the group consisting of SEQ ID NO: 34, SEQ ID NO: 35, SEQ ID NO: 36, SEQ ID NO: 37 or SEQ ID NO: 38.

According to some embodiments of the invention, the exogenous polynucleotide encodes a polypeptide consisting of the amino acid sequence set forth by any one of SEQ ID NOs: 6-30, or 39-58. According to some embodiments of the invention, the exogenous polynucleotide of the disclosure comprises a nucleic acid sequence encoding a polypeptide having an amino acid sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more say 100% homologous (e.g., identical) to the amino acid sequence selected from the group consisting of SEQ ID NOs: 6-30 or 39-58.

In some embodiments, the exogenous polynucleotide or the nucleic acid sequence further comprises at least one nuclear localization signal (NLS) sequence. In some embodiments, the exogenous polynucleotide or the nucleic acid sequence comprises 1, 2, 3, 4, 5, 6, 7, 8, 9, 10 NLS sequences or ranges in between. In some embodiments, the exogenous polynucleotide or the nucleic acid sequence comprises one NLS sequence. In some embodiments, the exogenous polynucleotide or the nucleic acid sequence comprises two NLS sequences. In some embodiments, the exogenous polynucleotide or the nucleic acid sequence comprises three NLS sequences.

In some embodiments, the NLS sequence is about or at least 5, 6, 7, 8, 9, 10, 11, 12, 13, 14, 15, 16, 17, 18, 19, 20, 25, 30, 35, 40, 45, 50, 60, 70, 80, 90 or 100 nucleotides long, or ranges in between. In some embodiments, the NLS sequence is about 10 nucleotides long. In some embodiments, the NLS sequence is about 11 nucleotides long. In some embodiments, the NLS sequence is about 15 nucleotides long. In some embodiments, the NLS sequence is about 16 nucleotides long. In some embodiments, the NLS sequence is about 21 nucleotides long. In some embodiments, the strength of NLS is weak. In some embodiments, the strength of NLS is strong. In some embodiments, the strength of NLS is weak, strong or combinations thereof.

The following Table 1 illustrates the amino acid locations of the NLS or predicted NLS and the relative strength thereof, when present.

TABLE 1

| SEQ ID No. | Name | Predicted NLS | Strength |
|---|---|---|---|
| 1 | hFTO | 8-18 | Weak |
| 2 | mhFTO | 8-18 | Weak |
| 3 | olFTO | 338-352 | Very weak |
| 4 | ssFTO | 8-18 | Weak |
| 5 | btFTO | 10-18 | Weak |
| 6 | apFTO | 18-33 | Strong |
| 7 | mcFTO | 3-45 | Strong |
| 8 | niFTO | 8-20, 537-551 | Strong |
| 9 | acFTO | 4-60, 495-512 | Strong |
| 10 | ctFTO | 2-38, 523-557 | Strong |
| 11 | crtFTO | 2-19 | Strong |
| 12 | cmFTO | None | None |
| 13 | mecFTO | 3-17 | Strong |
| 14 | anaFTO | 2-17 | Strong |
| 15 | pmFTO | 4-28 | Strong |
| 16 | arFTO | 51-67 | Strong |
| 17 | skFTO | 3-13 | Weak |
| 18 | otFTO | None | None |
| 19 | mpFTO | 10-40, 527-541 | Strong |
| 20 | esFTO-1 | 40-53, 85-95, 383-398, 652-715 | Strong |
| 21 | csFTO-2 | 5-31, 187-209, 486-502 | Strong |
| 22 | tpFTO | 3-31, 581-613 | Strong |
| 23 | pgFTO | 661-674 | Strong |
| 24 | pnmFTO | 4-18, 316-326, 514-549 | Strong |
| 25 | tmFTO | 78-84, 538-551, 588-610 | Strong |
| 26 | aeFTO | 4-56, 491-512 | Strong |
| 27 | aaFTO | 3-35, 505-518 | Strong |
| 28 | aiFTO | 4-25, 522-535 | Strong |
| 29 | asFTO | 4-70, 501-516 | Strong |

In the composition and methods of the present disclosure, the exogenous polynucleotide or a nucleic acid sequence disclosed herein are cloned into transformation vectors. Vectors used for plant transformation may include any vector that is generally know in in the art.

In some embodiments, the nucleic acid construct according to some embodiments of the invention comprises a promoter sequence and the isolated polynucleotide of some embodiments of the invention.

According to some embodiments of the invention, the isolated polynucleotide is operably linked to the promoter sequence. A coding nucleic acid sequence is “operably linked” to a regulatory sequence (e.g., promoter) if the regulatory sequence is capable of exerting a regulatory effect on the coding sequence linked thereto. As used herein, the term “promoter” refers to a region of DNA which lies upstream of the transcriptional initiation site of a gene to which RNA polymerase binds to initiate transcription of RNA. The promoter controls where (e.g., which portion of a plant) and/or when (e.g., at which stage or condition in the lifetime of an organism) the gene is expressed.

According to some embodiments of the invention, the promoter is heterologous to the isolated polynucleotide and/or to the host cell. As used herein the phrase “heterologous promoter” refers to a promoter from a different species or from the same species but from a different gene locus as of the isolated polynucleotide sequence. According to some embodiments of the invention, the isolated polynucleotide is heterologous to the plant cell (e.g., the polynucleotide is derived from a different plant species when compared to the plant cell, thus the isolated polynucleotide and the plant cell are not from the same plant species).

Any suitable promoter sequence can be used by the nucleic acid construct of the present invention. Preferably the promoter is a constitutive promoter, a tissue-specific, or an abiotic stress-inducible promoter. According to some embodiments of the invention, the promoter is a plant promoter, which is suitable for expression of the exogenous polynucleotide in a plant cell.

Suitable promoters for expression in wheat include, but are not limited to, Wheat SPA promoter (SEQ ID NO: 6590; Albanietal, Plant Cell, 9: 171-184, 1997, which is fully incorporated herein by reference), wheat LMW (SEQ ID NO: 6591 (longer LMW promoter), and SEQ ID NO: 6592 (LMW promoter) and HMW glutenin-1 (SEQ ID NO: 6593 (Wheat HMW glutenin-1 longer promoter); and SEQ ID NO: 6594 (Wheat HMW glutenin-1 Promoter); Thomas and Flavell, The Plant Cell 2:1171-1180; Furtado et al., 2009 Plant Biotechnology Journal 7:240-253, each of which is fully incorporated herein by reference), wheat alpha, beta and gamma gliadins [e.g., SEQ ID NO: 6595 (wheat alpha gliadin, B genome, promoter); SEQ ID NO: 6596 (wheat gamma gliadin promoter); EMBO 3:1409-15, 1984, which is fully incorporated herein by reference], wheat TdPR60 [SEQ ID NO: 6597 (wheat TdPR60 longer promoter) or SEQ ID NO: 6598 (wheat TdPR60 promoter); Kovalchuk et al., Plant Mol Biol 71:81-98, 2009, which is fully incorporated herein by reference], maize Ub1 Promoter [cultivar Nongda 105 (SEQ ID NO: 6599); GenBank: DQ141598.1; Taylor et al., Plant Cell Rep 1993 12: 491-495, which is fully incorporated herein by reference; and cultivar B73 (SEQ ID NO: 6600); Christensen, A H, et al. Plant Mol. Biol. 18 (4), 675-689 (1992), which is fully incorporated herein by reference]; rice actin 1 (SEQ ID NO: 6601; Mc Elroy et al. 1990, The Plant Cell, Vol. 2, 163-171, which is fully incorporated herein by reference), rice GOS2 [SEQ ID NO: 6602 (rice GOS2 longer promoter) and SEQ ID NO: 6603 (rice GOS2 Promoter): De Pater et al. Plant J. 1992: 2: 837-44, which is fully incorporated herein by reference], Arabidopsis Pho1 [SEQ ID NO: 6604 (arabidopsis Pho1 Promoter); Hamburger et al., Plant Cell. 2002; 14: 889-902, which is fully incorporated herein by reference], ExpansinB promoters, e.g., rice ExpB5 [SEQ ID NO: 6605 (rice ExpB5 longer promoter) and SEQ ID NO: 6606 (rice ExpB5 promoter)] and Barley ExpB1 [SEQ ID NO: 6607 (barley ExpB1 Promoter), Won et al. Mol Cells. 2010: 30:369-76, which is fully incorporated herein by reference], barley SS2 (sucrose synthase 2) [(SEQ ID NO: 6608), Guerin and Carbonero, Plant Physiology May 1997 vol. 114 no. 1 55-62, which is fully incorporated herein by reference], and rice PG5a [SEQ ID NO: 6609, U.S. Pat. No. 7,700,835, Nakase et al., Plant Mol Biol. 32:621-30, 1996, each of which is fully incorporated herein by reference].

Suitable constitutive promoters include. for example, CaMV 35S promoter [SEQ ID NO: 6610 (CaMV 35S (pQXNc) Promoter); SEQ ID NO: 6611 (PJJ 35S from Brachypodium); SEQ ID NO: 6612 (CaMV 35S (OLD) Promoter) (Odell et al., Nature 313:810-812, 1985)], Arabidopsis At6669 promoter (SEQ ID NO: 6613 (Arabidopsis At6669 (OLD) Promoter); see PCT Publication No. WO04081173A2 or the new At6669 promoter (SEQ ID NO: 6614 (Arabidopsis At6669 (NEW) Promoter)); maize Ub1 Promoter [cultivar Nongda 105 (SEQ ID NO: 6599); GenBank: DQ141598.1; Taylor et al., Plant Cell Rep 1993 12: 491-495, which is fully incorporated herein by reference; and cultivar B73 (SEQ ID NO: 6600): Christensen. A H. et al. Plant Mol. Biol. 18 (4), 675-689 (1992), which is fully incorporated herein by reference]; rice actin 1 (SEQ ID NO: 6601, McElroy et al., Plant Cell 2:163-171, 1990); pEMU (Last et al., Theor. Appl. Genet. 81:581-588, 1991); CaMV 19S (Nilsson et al., Physiol. Plant 100:456-462, 1997); rice GOS2 [SEQ ID NO: 6602 (rice GOS2 longer Promoter) and SEQ ID NO: 6603 (rice GOS2 Promoter), de Pater et al, Plant J November; 2(6):837-44. 1992]; RBCS promoter (SEQ ID NO: 6615): Rice cyclophilin (Bucholz et al, Plant Mol Biol. 25(5):837-43, 1994): Maize H3 histone (Lepetit et al, Mol. Gen. Genet. 231: 276-285, 1992); Actin 2 (An et al, Plant J. 10(1); 107-121, 1996) and Synthetic Super MAS (Ni et al., The Plant Journal 7: 661-76, 1995). Other constitutive promoters include those in U.S. Pat. Nos. 5,659,026, 5,608,149; 5,608,144: 5,604,121; 5,569,597: 5,466,785; 5,399,680; 5,268,463; and 5,608,142.

Suitable tissue-specific promoters include, but not limited to, leaf-specific promoters [e.g., AT5G06690 (Thioredoxin) (high expression, SEQ ID NO: 6616), AT5G61520 (AtSTP3) (low expression, SEQ ID NO: 6617) described in Buttner et al 2000 Plant, Cell and Environment 23, 175-184, or the promoters described in Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant Mol. Biol. 23:1129-1138, 1993; and Matsuoka et al., Proc. Natl. Acad. Sci. USA 90:9586-9590, 1993; as well as Arabidopsis STP3 (AT5G61520) promoter (Buttner et al., Plant, Cell and Environment 23:175-184, 2000)], seed-preferred promoters [e.g., Napin (originated from Brassica napus which is characterized by a seed specific promoter activity; Stuitje A. R. et. al. Plant Biotechnology Journal 1 (4): 301-309; SEQ ID NO: 6618 (Brassica napus NAPIN Promoter) from seed specific genes (Simon, et al., Plant Mol. Biol. 5. 191, 1985; Scofield. et al., J. Biol. Chem. 262: 12202, 1987; Baszczynski, et al., Plant Mol. Biol. 14: 633. 1990), rice PG5a (SEQ ID NO: 6609; U.S. Pat. No. 7,700,835), early seed development Arabidopsis BAN (AT1G61720) (SEQ ID NO: 6619. US 2009/0031450 A1), late seed development Arabidopsis ABI3 (AT3G24650) (SEQ ID NO: 6620 (Arabidopsis ABI3 (AT3G24650) longer Promoter) or 6621 (Arabidopsis ABI3 (AT3G24650) Promoter)) (Ng et al., Plant Molecular Biology 54: 25-38. 2004), Brazil Nut albumin (Pearson' et al., Plant Mol. Biol. 18: 235-245, 1992), legumin (Ellis, et al. Plant Mol. Biol. 10: 203-214, 1988), Glutelin (rice) (Takaiwa, et al., Mol. Gen. Genet. 208: 15-22, 1986; Takaiwa, et al., FEBS Letts. 221: 43-47, 1987), Zein (Matzke et al Plant Mol Biol, 143). 323-32 1990), napA (Stalberg, et al, Planta 199: 515-519, 1996), Wheat SPA (SEQ ID NO: 6590; Albanietal, Plant Cell, 9: 171-184, 1997), sunflower oleosin (Cummins, et al., Plant Mol. Biol. 19: 873-876, 1992)]. endospern specific promoters [e.g., wheat LMW (SEQ ID NO: 6591 (Wheat LMW Longer Promoter), and SEQ ID NO: 6592 (Wheat LMW Promoter) and HMW glutenin-1 [(SEQ ID NO: 6593 (Wheat HMW glutenin-1 longer Promoter)); and SEQ ID NO: 6594 (Wheat HMW glutenin-1 Promoter), Thomas and Flavell, The Plant Cell 2:1171-1180, 1990: Mol Gen Genet 216:81-90, 1989; NAR17:461-2), wheat alpha, beta and gamma gliadins (SEQ ID NO: 6595 (wheat alpha gliadin (B genome) promoter); SEQ ID NO: 6596 (wheat gamma gliadin promoter); EMBO 3:1409-15. 1984), Barley ltrl promoter, barley B1, C, D hordein (Theor Appl Gen 98:1253-62, 1999; Plant J 4:343-55, 1993; Mol Gen Genet 250:750-60. 1996), Barley DOF (Mena et al, The Plant Journal, 116(1): 53-62. 1998), Biz2 (EP99106056.7), Barley SS2 (SEQ ID NO: 6608 (Barley SS2 Promoter); Guerin and Carbonero Plant Physiology 114: 1 55-62, 1997), wheat Tarp60 (Kovalchuk et al., Plant Mol Biol 71:81-98, 2009), barley D-hordein (D-Hor) and B-hordein (B-Hor) (Agnelo Furtado. Robert J. Henry and Alessandro Pellegrineschi (2009)], Synthetic promoter (Vicente-Carbajosa et al., Plant J. 13: 629-640, 1998), rice prolamin NRP33, rice-globulin Glb-1 (Wu et al, Plant Cell Physiology 39(8) 885-889. 1998), rice alpha-globulin REB/OHP-1 (Nakase et al. Plant Mol. Biol. 33: 513-S22, 1997), rice ADP-glucose PP (Trans Res 6:157-68, 1997), maize ESR gene family (Plant J 12:235-46, 1997), sorgum gamma-kafirin (PMB 32:1029-35, 1996)], embryo specific promoters [e.g., rice OSH1 (Sato et al, Proc. Natl. Acad. Sci. USA, 93: 8117-8122), KNOX (Postma-Haarsma et al, Plant Mol. Biol. 39:257-71, 1999), rice oleosin (Wu et at, J. Biochem., 123:386, 1998)], and flower-specific promoters [e.g., AtPRP4, chalene synthase (chsA) (Van der Meer, et al., Plant Mol. Biol. 15, 95-109, 1990), LAT52 (Twell et al Mol. Gen Genet. 217:240-245; 1989), Arabidopsis apetala 3 (Tilly et al., Development. 125:1647-57, 1998), Arabidopsis APETALA 1 (AT1G69120, API) (SEQ ID NO: 6622 (Arabidopsis (AT1G69120) APETALA 1)) (Hempel et al., Development 124:3845-3853, 1997)], and root promoters [e.g., the ROOTP promoter [SEQ ID NO: 6623]; rice ExpB5 (SEQ ID NO: 6606 (rice ExpB5 Promoter); or SEQ ID NO: 6605 (rice ExpB5 longer Promoter)) and barley ExpB1 promoters (SEQ ID NO: 6607) (Won et al. Mol. Cells 30: 369-376. 2010); arabidopsis ATTPS-CIN (AT3G25820) promoter (SEQ ID NO: 6624; Chen et al., Plant Phys 135:1956-66, 2004); arabidopsis Pho1 promoter (SEQ ID NO: 6604, Hamburger et al., Plant Cell. 14: 889-902, 2002), which is also slightly induced by stress].

Suitable abiotic stress-inducible promoters include, but not limited to, salt-inducible promoters such as RD29A (Yamaguchi-Shinozalei et al., Mol. Gen. Genet. 236:331-340. 1993); drought-inducible promoters such as maize rab17 gene promoter (Pla et. al., Plant Mol. Biol. 21:259-266, 1993), maize rab28 gene promoter (Busk et. al., Plant J. 11:1285-1295. 1997) and maize Ivr2 gene promoter (Pelleschi et. al., Plant Mol. Biol. 39:373-380, 1999); heat-inducible promoters such as heat tomato hsp80-promoter from tomato (U.S. Pat. No. 5,187,267).

According to some embodiments of the invention, expressing the exogenous polynucleotide of the invention within the plant is effected by transforming one or more cells of the plant with the exogenous polynucleotide, followed by generating a mature plant from the transformed cells and cultivating the mature plant under conditions suitable for expressing the exogenous polynucleotide within the mature According to some embodiments of the invention, the transformation is effected by introducing to the plant cell one or more nucleic acid construct which include the exogenous polynucleotide of some embodiments of the invention and at least one promoter for directing transcription of the exogenous polynucleotide in a host cell (e.g., a plant cell).

The nucleic acid construct of some embodiments of the invention can further include an appropriate selectable marker and/or an origin of replication. According to some embodiments of the invention, the nucleic acid construct utilized is a shuttle vector, which can propagate both in E. coli (wherein the construct comprises an appropriate selectable marker and origin of replication) and be compatible with propagation in cells. The construct according to the present invention can be, for example, a plasmid, a bacmid, a phagemid, a cosmid, a phage, a virus or an artificial chromosome.

The nucleic acid construct of some embodiments of the invention can further include appropriate localization signals, such as nuclear localization signals (NLS). According to some embodiments of the invention, the nucleic acid construct utilized has a nuclear localization signal such as described in Raikhel, Plant Physiol. 100: 1627-1632 (1992) and references there, such as the SV40 long T antigen NLS [Kalderon et al. Cell 39: 499-509 (1984)]. The nuclear localization signal can be located anywhere in the protein but is conveniently located at the N-terminal end of the protein. As is illustrated in Table 1, there are predicted sites in the FTO homologs for the NLS>

The nucleic acid construct of some embodiments of the invention can be utilized to stably or transiently transform plant cells. In stable transformation, the exogenous polynucleotide is integrated into the plant genome and as such it represents a stable and inherited trait. In transient transformation, the exogenous polynucleotide is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principal methods of causing stable integration of exogenous DNA into plant genomic DNA include two main approaches:

(i) Agrobacterium-mediated gene transfer: Klee et al. (1987) Annu. Rev. Plant Physiol. 38:467-486: Klee and Rogers in Cell Culture and Somatic Cell Genetics of Plants. Vol. 6, Molecular Biology of Plant Nuclear Genes, eds. Schell. J., and Vasil. L. K., Academic Publishers. San Diego. Calif. (1989) p. 2-25; Gatenby, in Plant Biotechnology, eds. Kung, S. and Arntzen, C. J., Butterworth Publishers, Boston, Mass. (1989) p. 93-112.

(ii) Direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment. Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman. London, (1985) p. 197-209; and Ohta. Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. See, e.g., Horsch et al. in Plant Molecular Biology Manual AS, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledonous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

According to some embodiments of the invention, transgenic plants are generated by transient transformation of leaf cells, meristematic cells or the whole plant. Transient transformation can be affected by any of the direct DNA transfer methods described above or by viral infection using modified plant viruses.

Viruses that have been shown to be useful for the transformation of plant hosts include but not limited to, CaMV, Tobacco mosaic virus (TMV), brome mosaic virus (BMV) and Bean Common Mosaic Virus (BV or BCMV). Transformation of plants using plant viruses is described in U.S. Pat. No. 4,855,237 (bean golden mosaic virus: BGV), EP-A 67,553 (TMV), Japanese Published Application No. 63-14693 (TMV), EPA 194.809 (BV), EPA 278.667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants are described in WO 87/06261.

According to some embodiments of the invention, the virus used for transient transformations is avirulent and thus is incapable of causing severe symptoms such as reduced growth rate, mosaic, ring spots, leaf roll, yellowing, streaking, pox fonnation, tumor formation and pitting. A suitable avirulent virus may be a naturally occurring avirulent virus or an artificially attenuated virus. Virus attenuation may be affected by using methods well known in the art including, but not limited to, sub-lethal heating, chemical treatment or by directed mutagenesis techniques such as described, for example, by Kurihara and Watanabe (Molecular Plant Pathology 4:259-269, 2003), Gal-on et al. (1992), Atreya et al. (1992) and Huet et al. (1994).

Suitable virus strains can be obtained from available sources such as, for example, the American Type culture Collection (ATCC) or by isolation from infected plants. Isolation of viruses from infected plant tissues can be affected by techniques well known in the art such as described, for example by Foster and Taylor, Eds. “Plant Virology Protocols: From Virus Isolation to Transgenic Resistance (Methods in Molecular Biology (Humana Pr), Vol 81)”, Humana Press. 1998. Briefly, tissues of an infected plant believed to contain a high concentration of a suitable virus, preferably young leaves and flower petals, are ground in a buffer solution (e.g., phosphate buffer solution) to produce a virus infected sap which can be used in subsequent inoculations.

Construction of plant RNA viruses for the introduction and expression of non-viral exogenous polynucleotide sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; Takamatsu et al. FEBS Letters (1990) 269:73-76; and U.S. Pat. No. 5,316,931.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsulate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsulate the viral RNA.

According to an aspect of some embodiments of the invention, the method of increasing yield, biomass, growth rate, vigor, oil content, fiber yield, fiber quality, fiber length, photosynthetic capacity, abiotic stress tolerance, and/or nitrogen use efficiency of a plant, is effected by expressing within the plant an exogenous polynucleotide comprising a nucleic acid sequence encoding a polypeptide comprising an amino acid sequence selected from the group consisting of SEQ ID NOs: 6-30, and 39-58, thereby increasing the yield, biomass, growth rate, vigor, oil content, fiber yield, fiber quality, fiber length, photosynthetic capacity, abiotic stress tolerance, and/or nitrogen use efficiency of the plant.

According to an aspect of some embodiments of the invention, there is provided a method of increasing yield, biomass, growth rate, vigor, oil content, fiber yield, fiber quality, fiber length, photosynthetic capacity, abiotic stress tolerance, and/or nitrogen use efficiency of a plant, comprising expressing within the plant an exogenous polynucleotide comprising a nucleic acid sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 93%, at least about 94%, at least about 95%, at least about 96%, at least about 97%, at least about 98%, at least about 99%. e.g., 100% identical to the nucleic acid sequence selected from the group consisting of SEQ ID NOs: 34-38, thereby increasing the yield, biomass, growth rate, vigor, oil content, fiber yield, fiber quality, fiber length, photosynthetic capacity, abiotic stress tolerance, and/or nitrogen use efficiency of the plant.

In some embodiments, methods of the invention include all plants which belong to the superfamily Viridiplantae, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Cannabis ssp., Capsicum spp., Cassia spp., Centroema pubescens, Chacoomeles spp., Cinnamomum cassia, Coffea arabica, Colophospermum mopane, Coronillia varia, Cotoncaster scrotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalypfus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Gossypium spp., Grevillea spp., Guibourtia coleosperma. Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Linum spp., Linum usitatissimum, Loudetia simplex, Lotonus bainesli, Lotus spp., Macrotyloma axillare, Malus spp., Manihot esculenta, Medicago saliva. Metasequoia glyptostroboides. Musa sapientum, Nicotianum spp. Onobrychis spp., Ornithopus spp., Oryza spp., Peltophorum africanum, Pennisetum spp., Persea gratissima, Petunia spp., Phaseolus spp., Phoenix canariensis, Phonnium cookianum, Photinia spp., Picea glauca, Pinus spp., Pisum sativam, Podocarpus totara, Pogonarthria fleckii, Pogonaffiria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens. Sequoiadendron giganteum, Sorghum bicolor, Spinacia spp., Sporobolus fimbriatus. Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera. Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, maize, wheat, barley, rye, oat, peanut, pea, lentil and alfalfa, cotton, rapeseed, canola, pepper, sunflower, tobacco, eggplant. eucalyptus, a tree, an ornamental plant, a perennial grass and a forage crop. In some embodiments, algae and other non-Viridiplantae can be used for the methods of the present invention.

According to some embodiments of the invention, the plant used by the method of the invention is a crop plant such as rice, maize, wheat, barley, peanut, potato, sesame, olive tree, palm oil, banana, soybean, sunflower, canola, sugarcane, alfalfa, millet, leguminosae (bean, pea), flax, Lupinus, rapeseed, tobacco, poplar, and cotton.

In some embodiments, the plant used by the method is selected from the group consisting of canola, wheat, soy, millet, sorghum, cotton, tomato, grapes, peanuts, rice, lettuce, melon, sweet potato, carrot, cucumber, sugar cane, alfalfa, Camelina, or PennyCress.

In some embodiments, the FTO gene comprising an exogenous poluymileotide or a nucleic acid sequence comprising SEQ ID NO: 34, SEQ ID NO: 3, SEQ ID NO. 36. SEQ ID NO: 37 or SEQ ID NO: 38 is expressed in a nucleus of said plant, plant cell, tissue, or seed.

In some embodiments, the exogenous polynucleotide encodes a FTO polypeptide comprising the amino acid sequence set forth by any one of SEQ ID NOs: 6-30, or 39-71 is expressed in a nucleus of said plant, plant cell, tissue, or seed.

According to an aspect of some embodiments of the invention, there is provided a method of producing a plant having increased yield, growth rate, biomass, vigor, oil content, seed yield, fiber yield, fiber quality, fiber length, photosynthetic capacity, nitrogen use efficiency, drought tolerance and/or abiotic stress tolerance as compared to a wild type plant of the same species which is grown under the same growth conditions, the method comprising:
(a) transforming the plant with an exogenous polynucleotide encoding a polypeptide comprising an amino acid sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 93%, at least about 94%, at least about 95%, at least about 96%, at least about 97%, at least about 98%, at least about 99%, e.g., 100% homologous (e.g., having sequence similarity or sequence identity) to the amino acid sequence selected from the group consisting of SEQ ID NOs: 6-30, or 39-71, and
(b) selecting from the plants of step (a) a plant having increased yield, growth rate, biomass, vigor, oil content, seed yield, fiber yield, fiber quality, fiber length, photosynthetic capacity, nitrogen use efficiency, drought tolerance and/or abiotic stress tolerance (e.g., by selecting the plants for the increased trait).

According to an aspect of some embodiments of the invention, there is provided a method of selecting a transformed plant having increased yield, growth rate, biomass, vigor, oil content, seed yield, fiber yield, fiber quality, fiber length, photosynthetic capacity, nitrogen use efficiency, drought tolerance and/or abiotic stress tolerance as compared to a wild type plant of the same species which is grown under the same growth conditions, the method comprising:
(a) providing plants transformed with an exogenous polynucleotide encoding a polypeptide comprising an amino acid sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 93%, at least about 94%, at least about 95%, at least about 96%, at least about 97%, at least about 98%, at least about 99%, e.g., 100% homologous (e.g., having sequence similarity or sequence identity) to the amino acid sequence selected from the group consisting of SEQ ID NOs: 6-30, or 39-71,
(b) selecting from the plants of step (a) a plant having increased yield, growth rate, biomass, vigor, oil content, seed yield, fiber yield, fiber quality, fiber length, photosynthetic capacity, nitrogen use efficiency, drought tolerance and/or abiotic stress tolerance (e.g., by selecting the plants for the increased trait), thereby selecting the plant having increased yield, growth rate, biomass, vigor, oil content, seed yield, fiber yield, fiber quality, fiber length, photosynthetic capacity, nitrogen use efficiency, and/or abiotic stress tolerance as compared to the wild type plant of the same species which is grown under the same growth conditions.

According to an aspect of some embodiments of the invention, there is provided a method of selecting a transformed plant having increased yield, growth rate, biomass, vigor, oil content, seed yield, fiber yield, fiber quality, fiber length, photosynthetic capacity, nitrogen use efficiency, and/or abiotic stress tolerance as compared to a wild type plant of the same species which is grown under the same growth conditions, the method comprising:
(a) providing plants transformed with an exogenous polynucleotide at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 93%, at least about 94%, at least about 95%, at least about 96%, at least about 97%, at least about 98%, at least about 99%, e.g., 100% identical to the nucleic acid sequence selected from the group consisting of SEQ ID NOs: 34-38,
(b) selecting from the plants of step (a) a plant having increased yield, growth rate, biomass, vigor, oil content, seed yield, fiber yield, fiber quality, fiber length, photosynthetic capacity, nitrogen use efficiency, and/or abiotic stress tolerance,
thereby selecting the plant having increased yield, growth rate, biomass, vigor, oil content, seed yield, fiber yield, fiber quality, fiber length, photosynthetic capacity, nitrogen use efficiency, and/or abiotic stress tolerance as compared to the wild type plant of the same species which is grown under the same growth conditions.

According to some embodiments of the invention the plant is a dicotyledonous plant.

According to some embodiments of the invention the plant is a monocotyledonous plant.

According to some embodiments of the invention, there is provided a plant cell exogenously expressing the polynucleotide of some embodiments of the invention, the nucleic acid construct of some embodiments of the invention and/or the polypeptide of some embodiments of the invention.

In some embodiments, following stable transformation, plant propagation is exercised. In some embodiments, seed propagation is used. In some embodiments, the transformed plant is regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced from the seedlings to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

According to an aspect of some embodiments of the invention there is provided a method of growing a crop comprising seeding seeds and/or planting plantlets of a plant transformed with the exogenous polynucleotide of the invention, e.g., the polynucleotide which encodes the polypeptide of some embodiments of the invention, wherein the plant is derived from plants which have been transformed with the exogenous polynucleotide and which have been selected for at least one trait selected from the group consisting of increased abiotic stress tolerance, increased water use efficiency, increased growth rate, increased vigor, increased biomass, increased oil content, increased yield, increased seed yield, increased fiber yield, increased fiber quality, increased fiber length, increased photosynthetic capacity, and/or increased fertilizer use efficiency (e.g., increased nitrogen use efficiency) as compared to a non-transformed plant.

According to some embodiments of the invention, increased yield of corn may be manifested as one or more of the following: increase in the number of plants per growing area, increase in the number of ears per plant, increase in the number of rows per ear, number of kernels per ear row, kernel weight, thousand kernel weight (1000-weight), ear length/diameter, increase oil content per kernel and increase starch content per kernel.

According to some embodiments of the disclosure, the increase of plant yield can be determined by various parameters. For example, increased yield of rice may be manifested by an increase in one or more of the following: number of plants per growing area, number of panicles per plant, number of spikelets per panicle, number of flowers per panicle, increase in the seed filling rate, increase in thousand kernel weight (1000-weight), increase oil content per seed, increase starch content per seed, increased tiller number, among others. An increase in yield may also result in modified architecture or may occur because of modified architecture.

According to some embodiments of the disclosure, increased yield of soybean may be manifested by an increase in one or more of the following: number of plants per growing area, number of pods per plant, number of seeds per pod, increase in the seed filling rate, increase in thousand seed weight (1000-weight), reduce pod shattering, increase oil content per seed, increase protein content per seed, among others. An increase in yield may also result in modified architecture or may occur because of modified architecture.

According to some embodiments of the disclosure, increased yield of canola may be manifested by an increase in one or more of the following: number of plants per growing area, number of pods per plant, number of seeds per pod, increase in the seed filling rate, increase in thousand seed weight (1000-weight), reduce pod shattering, increase oil content per seed, among others. An increase in yield may also result in modified architecture or may occur because of modified architecture.

According to some embodiments of the disclosure, increased yield of cotton may be manifested by an increase in one or more of the following: number of plants per growing area, number of bolls per plant, number of seeds per boll, increase in the seed filling rate, increase in thousand seed weight (1000-weight), increase oil content per seed, improve fiber length, fiber strength, among others. An increase in yield may also result in modified architecture or may occur because of modified architecture.

In some embodiments, increased oil content can be determined by extraction of the oil from the seed or the vegetative portion of the plant. Briefly, lipids (oil) can be removed from the plant (e.g., seed) by grinding the plant tissue in the presence of specific solvents (e.g., hexane or petroleum ether) and extracting the oil in a continuous extractor. Indirect oil content analysis can be carried out using various known methods such as Nuclear Magnetic Resonance (NMR) Spectroscopy, which measures the resonance energy absorbed by hydrogen atoms in the liquid state of the sample [See for example, Conway T F. and Earle F R., 1963, Journal of the American Oil Chemists' Society; Springer Berlin/Heidelberg. ISSN: 0003-021X (Print) 1558-9331 (Online)]; the Near Infrared (NI) Spectroscopy, which utilizes the absorption of near infrared energy (1100-2500 in) by the sample; and a method described in WO/2001/023884, which is based on extracting oil a solvent, evaporating the solvent in a gas stream which forms oil particles, and directing a light into the gas stream and oil particles which forms a detectable reflected light.

Non-limiting examples of abiotic stress conditions include, salinity, osmotic stress, drought, water deprivation (e.g., drought), excess of water (e.g., flood, waterlogging), etiolation, low temperature (e.g., cold stress), high temperature, heavy metal toxicity, anaerobiosis, nutrient deficiency (e.g., nitrogen deficiency or nitrogen limitation), nutrient excess, atmospheric pollution and UV irradiation.

According to some embodiments of the disclosure, the method further comprises growing the plant expressing the exogenous polynucleotide under fertilizer limiting conditions (e.g., nitrogen-limiting conditions). Non-limiting examples include growing the plant on soils with low nitrogen content (40-50% Nitrogen of the content present under normal or optimal conditions), or even under sever nitrogen deficiency (0-10% Nitrogen of the content present under normal or optimal conditions), wherein the normal or optimal conditions include about 6-15 mM Nitrogen, e.g., 6-10 mM Nitrogen).

According to some embodiments of the disclosure, the method further comprises growing the plant expressing the exogenous polynucleotide under drought conditions (e.g., water depriving conditions). In some embodiments, drought conditions occur when the plant is not watered for about 2 days, about 4 days, about 6 days, about 7 days, about 10 days, about 20 days, about 30 days, about 60 days, or any number of days between 2 and 60 days. In some embodiments, the plants exogenously expressing the polynucleotide(s), the nucleic acid constructs and/or polypeptide(s) of the invention exhibit increased drought tolerance as compared to the plants not expressing the plants exogenously expressing the polynucleotide(s), the nucleic acid constructs and/or polypeptide(s) of the invention. In some embodiments, the methods of determining drought tolerance of the plant include, but not limited to measuring root length and wilting of leaves.

In some embodiments, once expressed within the plant cell or the entire plant, the level of the polypeptide encoded by the exogenous polynucleotide can be determined by methods well known in the art such as, activity assays, Western blots using antibodies capable of specifically binding the polypeptide. Enzyme-Linked Immuno Sorbent Assay (ELISA), radio-immuno-assays (RIA), immunohistochemistry, immunocytochemistry, immunofluorescence and the like or combinations thereof.

Methods of determining the level in the plant of the RNA transcribed from the exogenous polynucleotide are well known in the art and include, for example, Northern blot analysis, reverse transcription polymerase chain reaction (RT-PCR) analysis (including quantitative, semi-quantitative or real-time RT-PCR) and RNA-m situ hybridization or combinations thereof.

The sequence information and annotations uncovered by the present teachings can be harnessed in favor of classical breeding. Thus, sub-sequence data of those polynucleotides described above, can be used as markers for marker assisted selection (MAS), in which a marker is used for indirect selection of a genetic determinant or determinants of a trait of interest (e.g., biomass, growth rate, oil content, yield, abiotic stress tolerance, water use efficiency, nitrogen use efficiency and/or fertilizer use efficiency). Nucleic acid data of the present teachings (DNA or RNA sequence) may contain or be linked to polymorphic sites or genetic markers on the genome such as restriction fragment length polymorphism (RFLP), microsatellites and single nucleotide polymorphism (SNP), DNA fingerprinting (DFP), amplified fragment length polymorphism (AFLP), expression level polymorphism, polymorphism of the encoded polypeptide and any other polymorphism at the DNA or RNA sequence.

Examples of marker assisted selections include, but are not limited to, selection for a morphological trait (e.g., a gene that affects form, coloration, male sterility or resistance such as the presence or absence of awn, leaf sheath coloration, height, grain color, aroma of rice); selection for a biochemical trait (e.g., a gene that encodes a protein that can be extracted and observed; for example, isozymes and storage proteins); selection for a biological trait (e.g., pathogen races or insect biotypes based on host pathogen or host parasite interaction can be used as a marker since the genetic constitution of an organism can affect its susceptibility to pathogens or parasites).

In some embodiments, the polynucleotides and polypeptides described herein can be used in a wide range of economical plants, in a safe and cost effective manner. [0162] in some embodiments, the plant lines exogenously expressing the polynucleotide or the polypeptide of the invention are screened to identify those that show the greatest increase of the desired plant trait.

Thus, according to an additional embodiment of the present disclosure, there is provided a method of evaluating a trait of a plant, the method comprising: (a) expressing in a plant or a portion thereof the nucleic acid construct of some embodiments of the invention; and (b) evaluating a trait of a plant as compared to a wild type plant of the same type (e.g., a plant not transformed with the claimed biomolecules); thereby evaluating the trait of the plant.

According to an aspect of some embodiments of the disclosure there is provided a method of producing a crop comprising growing a crop of a plant expressing an exogenous polynucleotide comprising a nucleic acid sequence encoding a polypeptide at least about 80%, at least about 81%, at least about 82%, at least about 83%, at least about 84%, at least about 85%, at least about 86% at least about 87%, at least about 88%, at least about 89%, at least about 90%, at least about 91 0 at least about 92%, at least about 93%, at least about 94%, at least about 95%, at least about 96%, at least about 97%, at least about 98%, at least about 99%, or more say 100% homologous (e.g., identical) to the amino acid sequence selected from the group consisting of SEQ ID NOs: 34-38, wherein the plant is derived from a plant (parent plant) that has been transformed to express the exogenous polynucleotide and that has been selected for increased abiotic stress tolerance, increased water use efficiency, increased growth rate, increased vigor, increased biomass, increased oil content, increased yield, increased seed yield, increased fiber yield, increased fiber quality, increased fiber length, increased photosynthetic capacity, and/or increased fertilizer use efficiency (e.g., increased nitrogen use efficiency) as compared to a control plant, thereby producing the crop.

According to an aspect of some embodiments of the disclosure there is provided a method of growing a crop comprising seeding seeds and/or planting plantlets of a plant transformed with the exogenous polynucleotide of the invention, e.g., the polynucleotide which encodes the polypeptide of some embodiments of the invention, wherein the plant is derived from plants which have been transformed with the exogenous polynucleotide and which have been selected for at least one trait selected from the group consisting of increased abiotic stress tolerance, increased water use efficiency, increased growth rate, increased vigor, increased biomass, increased oil content, increased yield, increased seed yield, increased fiber yield, increased fiber quality, increased fiber length, increased photosynthetic capacity, and/or increased fertilizer use efficiency (e.g., increased nitrogen use efficiency) as compared to a non-transformed plant.

It is appreciated that certain features of the disclosure, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### Examples

Reference is now made to the following examples, which together with the above descriptions illustrate some embodiments of the invention in a non-limiting fashion.

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press. New York (1998): methodologies as set forth in U.S. Pat. Nos. 4,666.828: 4,683,202; 4,801,531; 5.192,659 and 5,272,057; “Cell Biology: A Laboratory Handbook”, Volumes I-III Cellis. J. E., ed. (1994); “Current Protocols in Immunology” Volumes I-III Coligan J. E., ed. (1994); Stites et al. (eds); available immunoassays are extensively described in the patent and scientific literature; “Nucleic Acid Hybridization” Hames, B. D., and Higgins S. J., eds. (1985); “Transcription and Translation” Hames, B. D., and Higgins S. J., Eds. (1984); “Immobilized Cells and Enzymes” IRL Press. (1986): “A Practical Guide to Molecular Cloning” Perbal, B., (1984); “PCR Protocols: A Guide to Methods and Applications”, Academic Press, San Diego, Calif. (1990); Marshak et al., “Strategies for Protein Purification and Characterization-A Laboratory Course Manual” CSHL Press (1996): all of which are incorporated by reference as if fully set forth herein. Other general references are provided throughout this document. The procedures therein are believed to be well known in the art and are provided for the convenience of the reader. All the information contained therein is incorporated herein by reference.

### Example 1: Obtaining cDNA of the FTO Gene

FTO genes were identified from the database of National Center for Biotechnology Information (NCBI. FTO Nucleic acid sequences (SEQ ID NOs: 31-38), and amino acid sequences (SEQ ID NOs: 1-30) were obtained. The corresponding cDNAs were purchased or synthesized by GenScript.

### Example 2: E. coli Expression of FTO

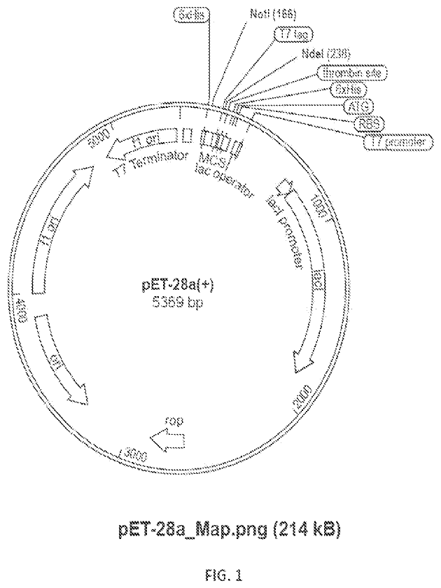

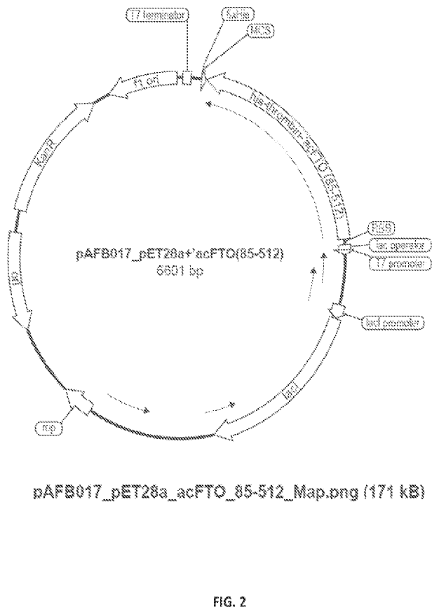

The FTO cDNAs (SEQ ID NOs: 1-9) were amplified after being cloned into the vector pET28a(+) using NdeI/NotI restriction sites. The gene of interest is under the regulation of the T7 promoter/lacI operator and inducible with IPTG. The average size of the inserted gene sequence was 510 bp. The plasmid used for cloning is illustrated in FIG. 1 and the pET-28a plasmid containing acFTO, SEQ ID No. 9 is illustrated in FIG. 2. E coli transformed with different FTO constructs disclosed herein were grown overnight at 37° C. At OD600, the culture was induced of 0.6 to 1.0 with 200 μl of 100 mM Isopropyl β-d-1-thiogalactopyranoside (IPTG) to each flask and moved to 16° C. with shaking at 80 rpm and incubated overnight. After approximately 18 hours, the culture was harvested by centrifugation at 4000K for 10 minutes. The supernatant was discarded, and pellets were harvested and frozen at −20° C. Samples of 1 ml were taken at induction and at harvest.

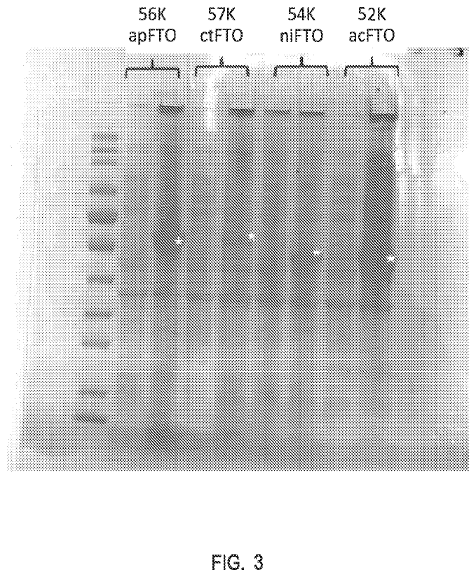

Pellets of the saved samples were resuspended in running buffer equivalent to OD (1 μl for each OD unit) and boiled for 10 minutes. Samples were run on a 4-20% gradient acrylamide gel at constant 140 mAmps. The resulting gel was stained with 50 ml of LabSafe™ GEL Blue for 20 minutes, de-stained overnight with water as exemplified in FIG. 3. As shown in FIG. 3, expression of various FTO homologs was observed upon induction with IPTG.

### Example 3: Protein Purification

The frozen cell pellets were resuspended in 10 ml of Lysis Buffer and lysed by passing through a Frech Press three times. Lysis Buffer was prepared by adding right before use, 5 mM beta-mercaptoethanol, lysozyme and protease inhibitor to 50 mM Tris-HCl, pH 8.0, 300 mM NaCl. Debris was removed by centrifuging at 4000 rpm for 10 min. The supernatant was transferred to a new tube and the pellet was discarded. NEBExpress® Ni Spin Columns (S1427L) were used to purify FTO protein following manufacturer's instructions. For each sample, five columns were run, and the eluates pooled, yielding 1 ml total of each. To concentrate and exchange the buffer for one more suitable for activity assays, Amicon Ultra-4 Centifugal Filer Devices with a molecular weight cut off of 10000K were used. The entire 1 ml sample of each purified enzyme was run over the column, spun at 4000 rpm for 20 minutes. Five milliliters of 50 mM Tris-HCl buffer, pH7.5 was added to the column and spun again at 4000 rpm for 15 minutes. The concentrated sample, about 200 ul each, was recovered from the column and frozen until use.

### Example 4: Detection of full-length FTO protein in Soy

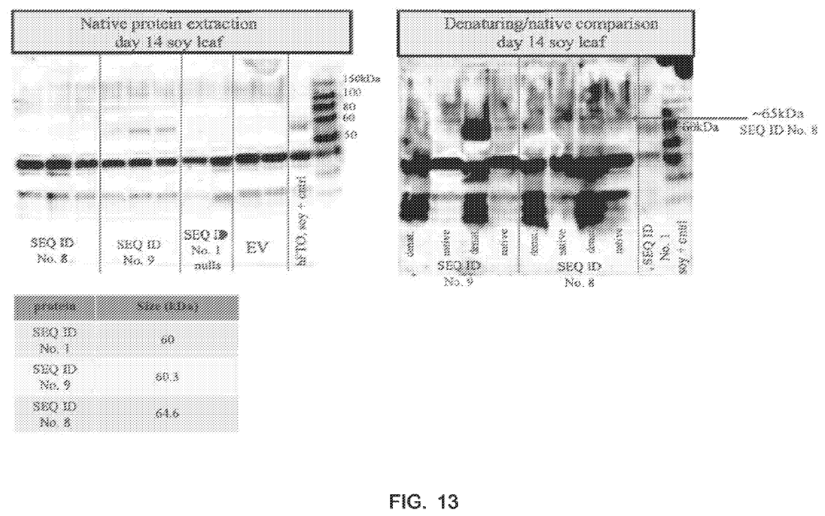

Constructs expressing hFTO homologs were transformed into soy plants. Samples of soy plants expressing hFTO (SEQ ID NO:1), acFTO (SEQ ID NO:9), niFTO (SEQ ID NO:8) were extracted and the FTO protein was detected by Western Blots shown in FIG. 13.

### Example 5: A. thaliana ecotype screening

Constructs expressing hFTO homologs were transformed into various A. thaliana ecotypes to determine if response differs between ecotypes. The following ecotypes were selected: Col-0 (standard), Ws-2 (standard), Lan-0 (Lansberg erecta) (standard). Some ecotypes having low freezing tolerance as defined by Maximilian Boinot et al. 2022.planta were also selected: C24 (Portugal 41° 15′00.0″N 8° 27′00.0″W), Can-0 (Canary Islands, low freezing tolerance), different M6A (Guan-Zheng Luo. Alice MacQueen et al. 2015 Nat. Commun.), Sah-0 (Sierra Alhambra—Spain). Other ecotypes having high freezing tolerance, as defined by Maximilian Boinot et al. 2022 planta were selected: Hen-16 (Northern Sweden, different M6A—Guan-Zheng Luo, Alice MacQueen et al. 2015 Nat. Commun.), Ms-0—Moscow (similar climate to Hen-16—Maximilian Boinot et al. 2022. Planta). Seeds from all ecotypes were stratified for 7 days at 4° C. in the dark. Eight (8) days later, the seeds were transferred to soil. Approximately two weeks later, plant growth was observed. Most ecotypes were observed to be germinating well (excepting Col-0 and Sah-0). Two days later new Col-0 seeds were stratified to sow two days later. Three weeks after observation, La-0 and C24 plants were cut back in preparation for floral dip. Agro (GV301) was streaked out with hFTO and mhFTO constructs. Two days later single colonies of hFTO and mhFTO were picked to grow on small scale. One day later they were transferred from small scale agro to large scale (125 mL flast with 75 mL LB). One day later Agro was spun down and resuspended in buffer with 1% sucrose and 500 μL/L silwet-77 (see Clough and Bent 1997 for details). This was placed in tupperware (plastic container) and floral parts were dipped in them, shaking off excess. One day later all plants were tied with string and kabob skewers.

After issues with Hen-16 and Can-0 plants, plants were restarted on ½ MS plates in a tissue culture chamber. Plants were exposed to 24 hours of continuous light in the growth chamber which resulted in stressed seedlings. The seedlings were transferred to soil in another chamber with standard A. thaliana growth conditions (16 hrs light/8 hrs dark 22 deg day, 19 deg night). Due to the error in light cycles, the Hen-16 plants which normally would require vernalization of several weeks at 4 deg C. and short days (8 hrs light/16 dark), ended up bolting early under the standard conditions.

This then allowed for the floral dipping of the Hen-16 plants with hFTO and the hFTO mutant in Agro. The plants were then tied up, staked and placed in seed collection bags to continue to grow. The primary bolts were cut back and 5 days later dipped. To help the plants recover from the cutting of the bolts a weak mix of water and fertilizer was applied to aid in fast recovery.

Unlike the Hen-16 plants, the Can-0 plants were not impacted by the erroneous growth conditions and took longer to form primary bolts. They were cut back and fertilized like the Hen-16 but the Can-0 plants took longer to recover and make lateral shoots with inflorescences.

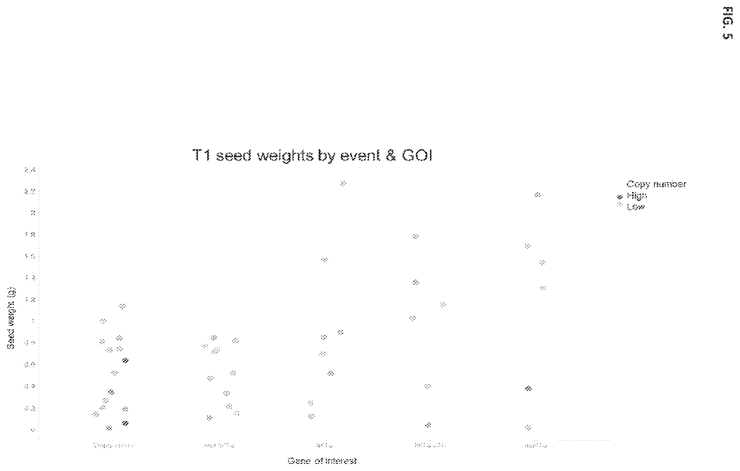

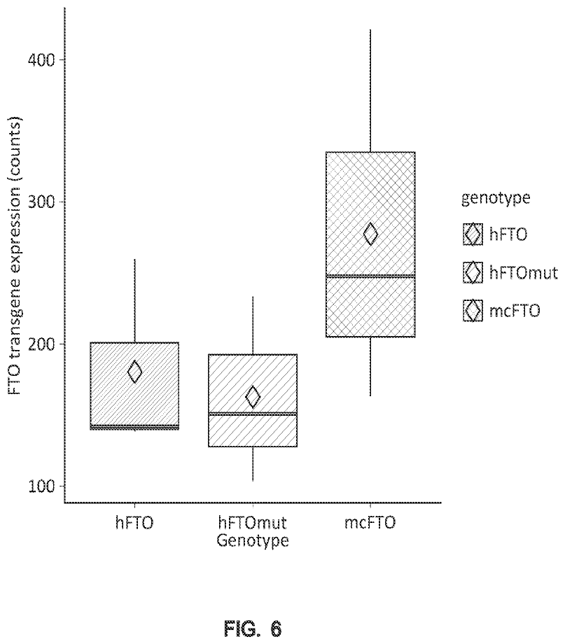

Harvested seeds were placed at 30° C. for 2 days in magenta boxes with dri-rite with the lid cracked open. After two days of drying, a 5% solution of liquid smoke was made to add to the seeds (Wright's hickory liquid smoke). Seeds were placed in mesh bags (NYLON BIOPSY BAG, 35×50 MM, 100—Electron Microscopy Services via VWR) and were sealed by folding and securing with tape tag and standard paper clip. Seed bags were then placed vertically in magenta boxes and ~75 mL of 5% liquid smoke added. They were then placed in the fume hood and allowed to soak overnight. Seeds were then removed from the solution and laid out on a bench pad to absorb liquid in the fume hood. After allowing the seeds to dry for 3 hours, they were placed in a ziplock bag with moist/damp sphagnum moss. The moss was wet but not saturated and most of the air was removed but not completely. Seeds were then placed in a box, taped shut to prevent light and put at 4° C. for a minimum of 2 weeks. Afterward, seeds could be planted in soil or kept in a seed dryer at 30 C for 2-3 days followed by storing in envelopes for a few months longer before planting. Seeds retained a 95% germination rate after a few years of storage. Seeds were categorized by copy number and weight as shown in FIG. 5.

### Example 6: Seed Weight of Seteria Viridis Transformed with FTO

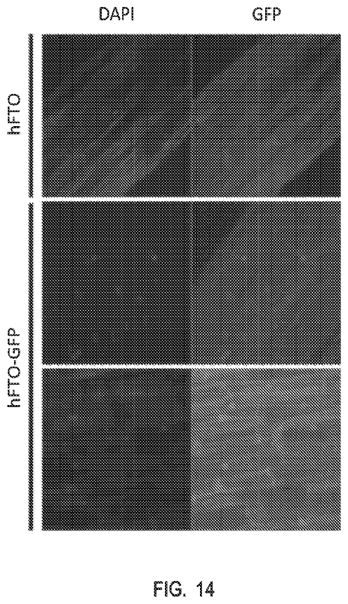

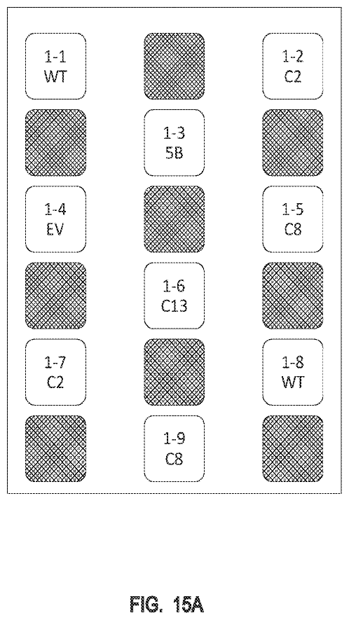

Constructs expressing hFTO homologs were transformed into Seteria viridis plants. To analyze the expression of FTO in S. viridis, S. virdis plants expressing GFP tagged hFTO was processed for DAPI and GFP visualization. As illustrated in FIG. 14, hFTO-GFP was detected in nuclei.

### Example 7: Root Assay on Seteria viridis

Next, root growth of transgenic Seteria virdis plants was determined by comparing each of the mcFTO expressing plants along will null and empty vectors. Seteria seeds that were potted in soil, were returned to 28° C. in magenta boxes with dri-rite to prevent spurious germination of the seeds within in the mesh bags. 4 days later, 10-12 seeds were placed in 1.5 mL epi tubes with 0.1% Triton-X and 20% volume bleach for surface sterilization. This was. Seeds were placed on the rocker for 20 minutes to sterilize. The Triton-X and bleach solutions were washed off and followed by 8 washes with sterile autoclaved diH2O. As the seeds were washed with the water, they began to imbibe the water and produce mucilage and protrude their radicals. This allowed for individual seeds to be pulled out of the water and placed into the pouches. Pouches were moistened with 15 mL of ½MS media and squished to ensure all of the pouch material was wet. With forceps, each seed was individually placed in a notch to allow for germination and plant growth. Once complete, the pouches were placed in the Conviron growth chamber with the following settings: 450 umol/s light intensity, 12 hours light/12 hours dark, 30° C. during the day and 22° C. at night. Pouches were checked daily to determine if any more ½MS should be added to the samples for watering. This usually was ~5 mL every few days. At day 14, the primary root was traced with a black sharpie marker. The pouch was then backlight with a white lightbox and photographed for measurement in ImageJ software. Root length was determined and plotted as illustrated in FIGS. 4 and 7.

### Example 8: Analysis ATACseq (Assay for Transposase-Accessible Chromatin with Sequencing

For determining regions of accessible chromatin, ATAC sequence analysis was performed. The nf-core ATACseq (Assay for Transposase-Accessible Chromatin using sequencing) pipeline doi: 10.5281/zenodo.2634132 was used to analyze ATACseq sequencing data following these steps:
1. Raw sequencing read Quality Control (FastQC)
2. Sequencing adapter trimming (Trim Galore!)
3. Trimmed reads aligned to reference genome (BWA)
4. Duplicate reads counted and marked (picard)
5. Call broad peaks, or regions of accessible chromatin based of read pileups (MACS2)
6. Annotate peaks relative to gene features (HOMER)—Create consensus peak set across all samples and create tabular file to aid in the filtering of the data (BEDTools)
   Count reads in consensus peaks relative to merged library-level alignments (featureCounts)
   Differential accessibility analysis, PCA and clustering (R, DESeq2)
7. Present QC for raw read, alignment, peak-calling and differential accessibility results (ataqv, MultiQC, R)

The results of the ATAC analysis are provided in Table 2 below. As shown in Table 2, a majority of peaks that are unique to mcFTO plants are in intergenic regions (between protein coding genes, may contain regulatory elements).

TABLE 2

| | mcFTO | hFTOmut | Shared |
|---|---|---|---|
| Intergenic | 2633 | 620 | 6948 |
| Exon | 484 | 952 | 5732 |
| Promoter - Transcription Start Site | 461 | 742 | 10006 |
| Transcription Termination Site | 303 | 573 | 4549 |
| Intron | 216 | 511 | 2174 |
| Other | 2 | 0 | 0 |
| Total | 4099 | 3398 | 29409 |

### Example 9: Soy Drought Assay #1

For this assay, construct containing AFN008 (Seq. ID No. 9) was transformed into a vector for use, pAFB070—[pSOLIS21-070]. The empty vector was the binary vector pSOLIS21 (a Solis specific vector). Positive transformants were moved to soil and seeds (T1 seeds) were collected from plants. T1 seeds were then planted and genotyped for the presence of AFN008 using a copy number assay. GOI positive plants were transplanted into larger pots to generate seeds (T2 seeds). Of the events shown above the T1 parent plant was used to generate T2 seed. T2 seeds were used in the drought assay. The growth conditions of the plants were (14 hrs light/10 hrs dark 25° C. day, 23° C. night). The plants were grown on Berger BM7-35% soil with bark high porosity and were fertilized on Monday, Wednesday and Friday of every week with Nitrogen:Phosphorous:Potassium (NPK) fertilizer in the ratio 15-16-17.

For the assay, 1801 trays were filled with BM7-35 soil and saturated. 1 seed per cell was planted at around 1-1.5 inches deep. Each cell had a stake label with the individual seed information. At the same time, WT Illini-3025N seed was sown to create a border/buffer row around all of the experimental soy flats.

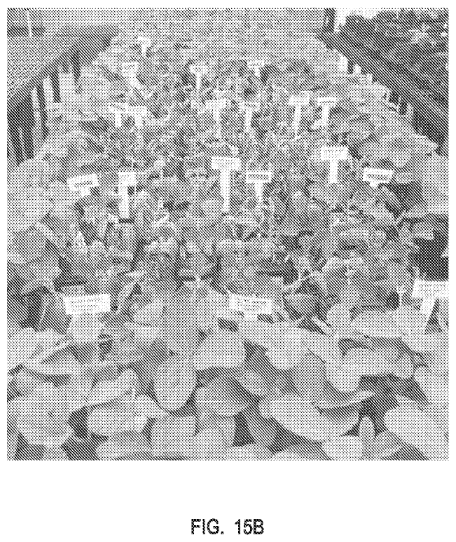

Between day 12-13, the newest leaf was clipped to collect tissue for DNA extraction and copy number analysis. The plants were allowed to grow until day 17 and drought was started for half of the plants. The well-watered and drought plants were put into 1801 flats, and checkerboarded to allow for gaps between the plants as shown below. 10 flats were generated using a Random Complete Block Design (RCBD) for each of the events along with the controls. Each event had 15 plants per treatment spread among the 10 flats. 10 flats were well-watered, and 10 flats were put under total drought conditions with all water withheld for the entire process. The exact positions were determined for the plants and each map used to place the correct plant in the right position for the experiment as shown in FIG. 15A. FIG. 15B shows the well-watered and droughted plants surrounded by the soy border to prevent edge effects and additional (non-drought related water loss). After placing the plants into the RCBD, two photos were taken of the pots from the trays. There was an aerial picture that showed the entire flat for each tray. A side view was also taken where each pot was lined up by genotype to capture the shoot. Photos were then taken every day, an aerial and side view for the next 7 days. After taking a final aerial photo of the plants on Day 7 of drought treatment, all the roots were washed to remove as much soil as possible. Five plants were laid down for an aerial picture with scale and color correction card to capture the root morphology. The total root length was also manually measured and recorded.

### Determination of Wilt Score

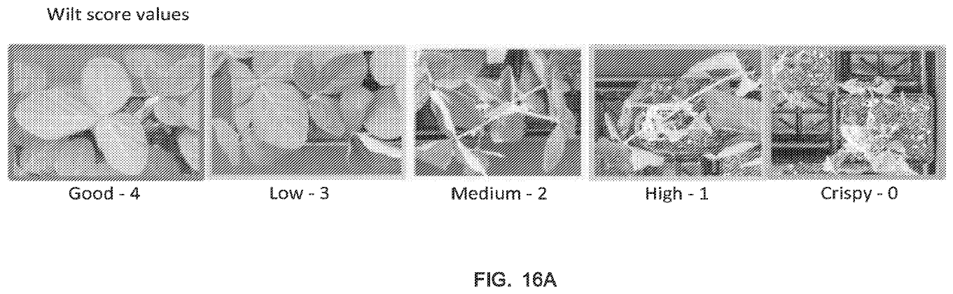

Aerial photos from the final day of drought were then examined independently by two people. The scoring system was as follows; good—4, low—3, medium—2, high—1, crispy—0 and as illustrated in FIG. 16A. Using the RCBD map, the score for each individual plant was recorded based on appearance. The higher the score the less wilt, the lower the score the greater the degree of wilting. The individual scores were averaged for each plant and then combined for an event specific average wilt score. The standard deviation was also calculated for each average event. To determine the significance of the wilt, a t-test was used to compare to the empty vector plants wilting values.

### Measurement of Root Length

Next, the length of each individual root was measured and recorded per plant. The results for each event were averaged for treatment either well-watered or drought. Standard derivation was determined. To determine significance, a t-test was used to compare the empty vector plants to AFN008 plants. The results of the root length are depicted in FIG. 16B. The star indicates an event where it is statistically significant from the empty vector plants with a p value of less than 0.05.

### Example 10: Soy Drought Assay #2

For this assay, constructs containing AFN008 (acFTO, Seq ID No. 9) was transformed into a vector for use, pAFB070—[pSOLIS21-070]. The empty vector was the binary vector pSOLIS21 (a Solis specific vector). Positive transformants were moved to soil and seeds (T1 seeds) were collected from plants. T1 seeds were then planted and genotyped for the presence of AFN008 using a copy number assay. GOI positive plants were transplanted into larger pots to generate seeds (T2 seeds). T1 parent plant was used to generate T2 seed. T2 seeds were used in the drought assay. For AFN008m the initial plants for the drought assay are shown planted in the 1801 flats on the day of sowing. The growth conditions of the plants were (14 hrs light/10 hrs dark 25° C. day. 23° C. night). The plants were grown on Berger BM7-35% soil with bark high porosity and were fertilized on Monday, Wednesday and Friday of every week with Nitrogen:Phosphorous:Potassium (NPK) fertilizer in the ratio 15-16-17.

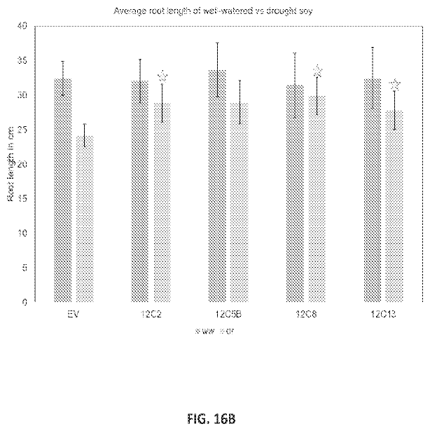

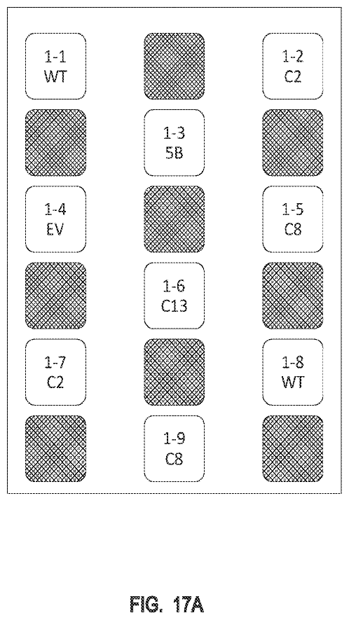

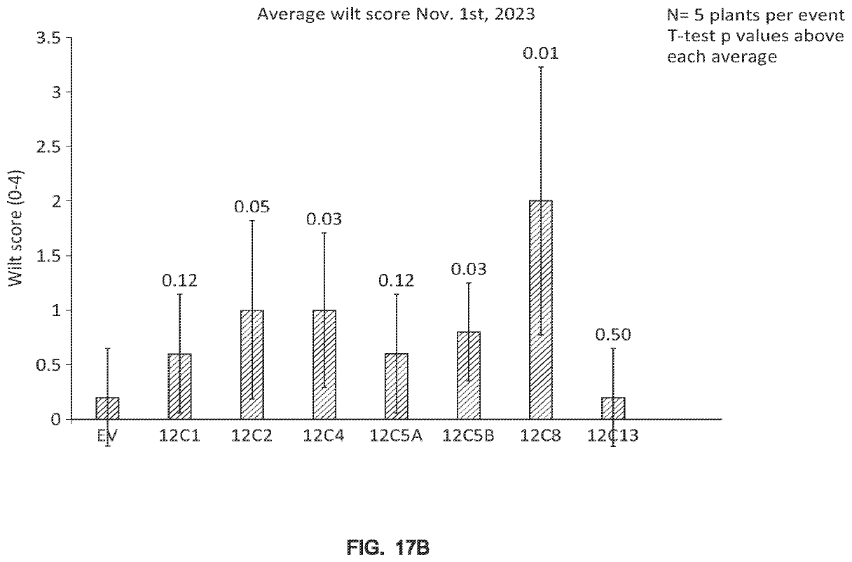

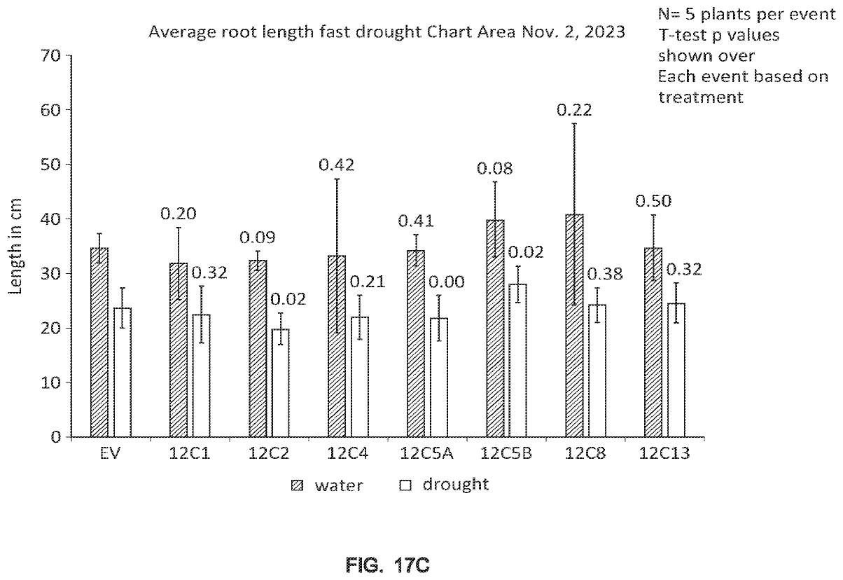

For this assay, 1801 trays were filled with BM7-35 soil and saturate. 1 seed per cell was planted at around 1-1.5 inches deep. Each cell had a stake label with the individual seed information. Between day 16-18, clipped the newest leaf to collect tissue for DNA extraction and copy number analysis. The plants were allowed to grow until day 18. Drought was started for half of the plants. The well-watered and drought plants were put into 1801 flats, and checkerboarded to allow for gaps between the plants as shown here. A total of 10 flats were generated using a Random Complete Block Design (RCBD) for each of the events along with the controls. Each event had 5 plants per treatment spread among the 10 flats. 10 flats were well-watered, and 10 flats were put under total drought conditions with all water withheld for the entire process. The exact positions were determined for the plants and each map used to place the correct plant in the right position for the experiment as shown in FIG. 17A. After placing the plants into the RCBD, two photos were taken of the pots from the trays. There was an aerial picture that showed the entire flat for each tray. A side view was also taken where each pot was lined up by genotype to capture the shoot. Photos were then taken every day, an aerial and side view for the next 7 days. After taking a final aerial photo of the plants on Day 7 of drought treatment, all the roots were washed to remove as much soil as possible. Five plants were laid down for an aerial picture with scale and color correction card to capture the root morphology. The total root length was also manually measured and recorded.

### Determination of Wilt Score

Aerial photos from the final day of drought were examined independently by two people. The scoring system was as follows; good—4, low—3, medium—2, high—1, crispy—0 as illustrated in FIG. 16A. Using the RCBD map, the score for each individual plant was recorded based on appearance. The higher the score the less wilt, the lower the score the greater the degree of wilting. The individual scores were averaged for each plant and then combined for an event specific average wilt score. The standard deviation was also calculated for each average event. To determine the significance of the wilt, a t-test was used to compare to the empty vector plants wilting values. Wilt score results are represented in FIG. 17B.

### Measurement of Root Length

The length of each individual root was measured and recorded per plant. The results for each event were averaged for treatment either well-watered or drought. Standard derivation was determined as well. To determine significance, a t-test was used to compare the empty vector plants to AFN008 plants. The results of the root length are depicted in FIG. 17C. The star indicates an event where it is statistically significant from the empty vector plants with a p value of less than 0.05.

### Example 11: Sov Drought Assay #3

For this assay, constructs containing AFN004 (mcFTO, Seq. ID No. 7), AFN007 (niFTO, Seq. ID No. 8), and AFN011 (mutant control for AFN004) were transformed into a vector for use by Solis. At the time of transformation, empty vector controls were created for each transformation instance. The mutant control of AFN004 has R321Q and R327Q substitutions.

Positive transformants were moved to soil and seeds (T1 seeds) were collected from plants. T1 seeds were then planted and genotyped for the presence of AFN008 using a copy number assay. GOI positive plants were transplanted into larger pots to generate seeds (T2 seeds). T1 parent plant was used to generate T2 seed. T2 seeds were used in the drought assay. For AFN008m the initial plants for the drought assay are shown planted in the 1801 flats on the day of sowing. The growth conditions of the plants were (14 hrs light/10 hrs dark 25° C. deg day, 23° C. deg night). The plants were grown on Berger BM7-35% soil with bark high porosity and were fertilized on Monday, Wednesday and Friday of every week with Nitrogen:Phosphorous:Potassium (NPK) fertilizer in the ratio 15-16-17. Seeds were planted, genotyped for the presence of the construct using a copy number assay specific for either the promoter region or the antibiotic resistance region. GOI positive plants were selected to continue in the drought assay—with the exception of AFN004 event 570, which contained no plants containing the construct, but was carried forward anyway. Table 3 contains information on the number of plants per treatment carried forward in the drought experiment. For AFN004, AFN011, and AFN004_EV controls, only T1 seeds were available, so were utilized in this experiment. For AFN007 and AFN007_EV. T2 seeds were available for use in this experiment, therefore parent plant information is noted for AFN007 events and EV controls only.

TABLE 3

| | | | | # of seeds | | | |
|---|---|---|---|---|---|---|---|
| | | | | planted for | | | |
| Species | Genotype | Event | Parent Plant | drought exp | # germinated | # positive | #nulls |
| Glycine max | AFN004 | 567 | na(T1) | 20 | 20 | 17 | 3 |
| Glycine max | AFN004 | 570 | na(T1) | 20 | 20 | 0 | 17 |
| Glycine max | AFN004 | 575 | na(T1) | 20 | 18 | 16 | 2 |
| Glycine max | AFN004 | 582 | na(T1) | 20 | 19 | 15 | 4 |
| Glycine max | AFN004_EV | 535 | na(T1) | 20 | 18 | Na | Na |
| Glycine max | AFN011 | 935 | na(T1) | 20 | 20 | 15 | 5 |
| Glycine max | AFN011 | 939 | na(T1) | 20 | 14 | 13 | 1 |
| Glycine max | AFN007 | 11B25 | 9 | 45 | 45 | 29 | 16 |
| Glycine max | AFN007 | 11B19B | 25 | 45 | 40 | 24 | 15 |
| Glycine max | AFN007 | 11B19A | 20 | 45 | 43 | 29 | 12 |
| Glycine max | AFN007_EV | 11A2 | 33 | 40 | 38 | na | na |
| Glycine max | AFN007_EV | 11A2 | 36 | 40 | 37 | na | na |

| | | Field | | | | | | # in |
|---|---|---|---|---|---|---|---|---|
| | | copy # | #no | Germination | % | | Total # | experiment |
| Species | Genotype | rxn | germination | success % | positives | % nulls | planted | per trtmnt* |
| Glycine max | AFN004 | 0 | 0 | 100 | 85.0 | 15.0 | 20 | 7 |
| Glycine max | AFN004 | 3 | 0 | 100 | 0.0 | 100.0 | 20 | 7 |
| Glycine max | AFN004 | 0 | 2 | 90 | 88.9 | 11.1 | 20 | 7 |
| Glycine max | AFN004 | 0 | 1 | 95 | 78.9 | 21.1 | 20 | 7 |
| Glycine max | AFN004_EV | na | 2 | 90 | na | na | 20 | 7 |
| Glycine max | AFN011 | 0 | 0 | 100 | 75.0 | 25.0 | 20 | 7 |
| Glycine max | AFN011 | 0 | 6 | 70 | 92.9 | 7.1 | 20 | 7 (drought) |
| | | | | | | | | 6 (well-watered) |
| Glycine max | AFN007 | 0 | 0 | 100.0 | 64.4 | 35.6 | 45 | 14 |
| Glycine max | AFN007 | 1 | 5 | 88.9 | 61.5 | 38.5 | 45 | 12 |
| Glycine max | AFN007 | 2 | 2 | 95.6 | 70.7 | 29.3 | 45 | 14 |
| Glycine max | AFN007_EV | na | 2 | 95 | na | na | 40 | 7 |
| Glycine max | AFN007_EV | na | 3 | 92.5 | na | na | 40 | 8 |

For planting, 1801 trays were filled with BM7-35 soil and saturated with water. 1 seed per cell was planted at a depth of 1-1.5 inches. Each cell was labelled with a tag indicating individual seed information. At the same time as experimental plants were seeded, WT IL3025N seeds were planted to create a WI border to surround experimental flats and lessen positional effects and drying out of experimental plants on the edges.

Copy number assay s were performed to determine which plants contain gene of interest. Between day 12-13, a portion of the newest leaf was collected for DNA extraction and copy number analysis. Copy #analysis was used to determine nulls (i.e., plants not containing the gene of interest) and positive (i.e., plants containing the gene of interest) plants. This information was used to cull the null plants and only positive plants moved forward into the drought experiment (for this experiment, the exception was AFN004 event 570, which only contained null plants and was carried forward).

Plants for the experiment were grown for 17 days under normal conditions (i.e., normal watering and fertilizing). After 17 days of growth under normal conditions, plants were split into two treatments: droughted (i.e., complete withholding of water) and well-watered (i.e., normal-watering schedule).

The well-watered and drought plants for each event separated and put into 1801 flats in a checkerboard pattern to allow for gaps between the plants as shown above. Plants within each treatment were arranged in a Randomized Complete Block Design (RCBD) along with controls to reduce positional effects. Additionally, a border of WT plants surrounded the greenhouse bench to prevent border plants from drying out more than plants in an internal position. The number of plants per treatment for each construct/event is shown in Table 3.

Half of the plants per event were well-watered and half per event were put under total drought conditions (all water was withheld) for an additional 6 or 7 days. Droughted plants were grown for 6 days without water or fertilizer and were measured and sampled on the 6^{th} day of drought. Well-watered plants were grown for 7 days under normal conditions and measured and sampled on the 7^{th} day (see below for measuring and sampling protocols). Wilt scores were determined post-measuring and sampling from photographs.

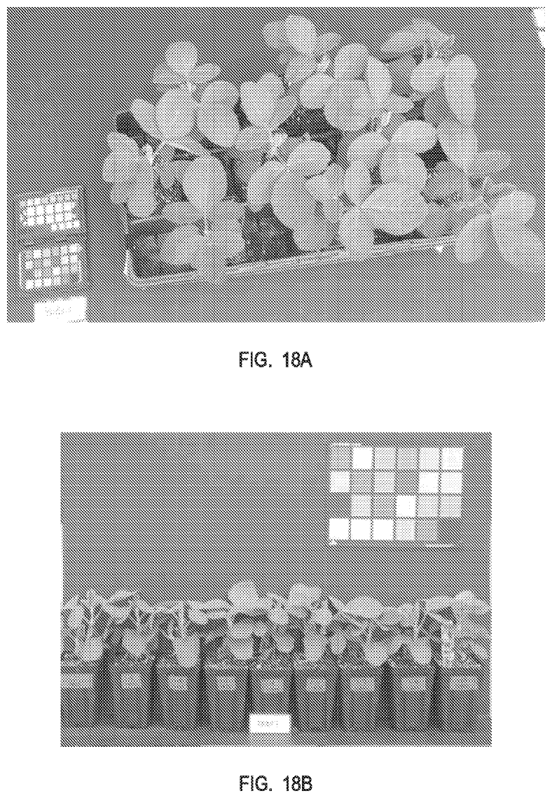

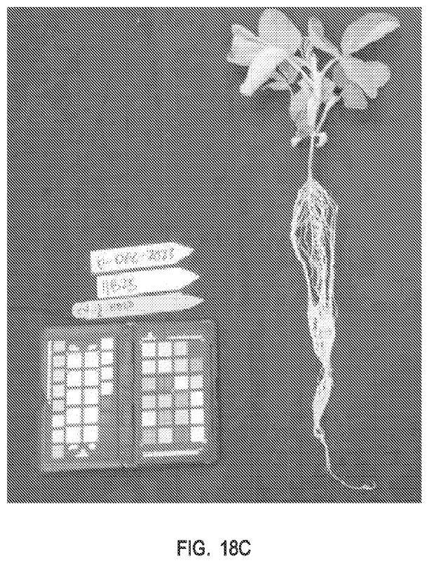

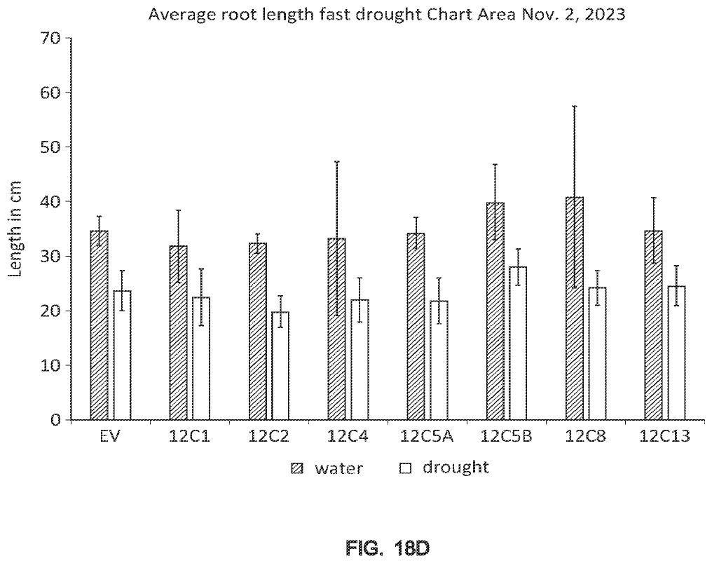

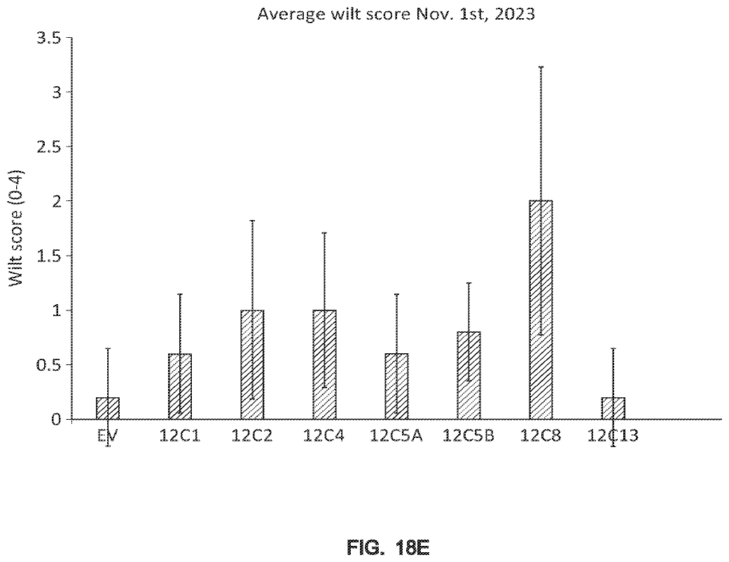

After the beginning of the droughting period, photos were taken of the experimental (droughted and well-watered) plants daily. This included an overhead photo of each tray, as well as a photo of all the plants per tray from a side view as illustrated in FIG. 18B. Examples of each photo type are illustrated in FIGS. 18A and 18B. Two photos were taken of the pots from the trays. Photos were taken through day 6 and day 7 for the droughted and well-watered plants, respectively.

Measuring and Sampling: On day 6 droughted plants were taken for measuring and sampling. Roots were washed to remove soil. Root length was measured from the base of the plant to the tip of the root and record in cm. Total aerial height was measured and recorded for each plant as the length (cm) from the top of the root to the top of the stem. These values were added to give a total plant size for analysis. Plants placed on black velvet for an aerial picture with scale and color correction card to capture root morphology as illustrated in FIG. 18C. Photos were used to determine total root area (cm2) as described below. Additionally, 4 plants per construct/event per treatment were sampled on liquid nitrogen and stored on dry ice for subsequent molecular analyses.

Total root area was determined using the program PlantCV. The software was used to split the image into color spaces and then a subsequent mask for the root tissue. The final root mask after cleaning was run through the “pcv.analze.shape” function. The “pcv.analze.shape” is able to measure in pixels outputs such as, total area, longest path, width, height and other metrics. The main measurement that was used was the “principle measurement”. The images were processed as .csv file outputs with further analyses performed in R. The area measures were converted from pixels to cm2 and allowed for the data to be normalized to compensate for differences between individual images. The normalized data were then analyzed using an ANOVA to compare the differences in the root mean area between groups with a Tukey HSD correction.

### Determination of Wilt Score

Aerial photos from the final day of drought were examined independently by two people, using the following scoring system: good—4, low—3, medium—2, high—1, crispy—0 as illustrated in FIG. 16A. Using the RCBD map, the score for each individual plant was recorded based on appearance. The higher the score the less wilt, the lower the score the greater the degree of wilting. The individual scores were averaged for each plant and then combined to give an average wilt score for each event for droughted plants. The standard deviation was also calculated for each average event. The results of the wilt score are depicted in FIG. 18E.

The measurements, including root length (cm), root area (cm2), aerial height (cm), total plant height (cm), as well as wilt score, were averaged for an event for data analysis. Standard deviation for each measurement within an event was calculated. Droughted events were compared to their well-watered counterparts. Events in both treatments were compared to both empty vector and mutant (for ANF004) controls to determine effect of the presence of the FTO homolog. Results are presented below in Table 4. The plants were photographed as shown in FIG. 18D. Overall results were summarized into a decision table as illustrated in Table 4 to compare indifference to drought, performance under drought, performance under well-watered (“normal”) conditions, and wilt score. Green indicates a favorable result for that event.

TABLE 4
Decision Table

| | | | | | | Performance under drought | | | |
|---|---|---|---|---|---|---|---|---|---|
| | | Indifference to Drought | | | | | | D total | |
| | | | | | Root | D Roots | D shoots | Plan | D Root |
| | | Root | Shoot | Total | surface | Longer | taller | Bigger | surface |
| Plant Information | | Length | height | Plan (cm) | area | than | than | than | area |
| Genotype | Event | (cm) DI | (cm DI | DI | (cm2) DI | controls | controls | Controls | (cm2) |
| ni | 11B19A | N | N | N | N | N | Y | N | N |
| | 11B19B | Y | N | N | N | Y | N | Y | N |
| | 11B25 | N | N | N | N | Y | Y | Y | Y |
| mc | 567 | Y | Y | Y | N | Y | Y | Y | N |
| | 570 | N | N | N | N | N | N | N | N |
| | (all nulls) | | | | | | | | |
| | 575 | N | Y | N | N | Y | Y | Y | N |
| | 582 | N | Y | N | N | Y | Y | Y | N |

Decision Table

| | | Performance under “normal” | | | | |
|---|---|---|---|---|---|---|
| | | | | | ND Root | |
| | | ND Roots | ND Shoots | ND Total | surface area | Wilt score |
| Plant Information | | Longer than | taller than | Plant bigger | (cm2) - | better than |
| Genotype | Event | Controls | controls | than controls | Imaging | controls |
| ni | 11B19A | N | N | N | N | Y |
| | 11B19B | N | N | N | N | Y |
| | 11B25 | Y | Y | Y | N | Y |
| mc | 567 | N | N | N | N | Y* |
| | 570 | Y | Y | Y | N | Y* |
| | (all nulls) | | | | | |
| | 575 | Y | N | Y | N | Y* |
| | 582 | Y | N | Y | N | Y* |

*mut 939 had highest wilt score; evaluation does not include 939

1. A plant comprising plant cells comprising a nucleic acid molecule encoding a fat mass and obesity-associated factor (FTO) gene, wherein the expression of the said nucleic acid molecule results in one or more of increased yield, biomass, growth rate, drought tolerance and abiotic stress tolerance relative to a plant of same variety lacking the nucleic acid molecule encoding the FTO gene.

2. The plant of claim 1, wherein said FTO nucleic acid molecule is from vertebrates, invertebrates, algae, or oomycetes.

3. The plant of claim 1 wherein the nucleic acid molecule encoding an FTO that has at least 80% identity to any one of SEQ ID NOs: 34-38.

4. The plant of claim 1, wherein the plant exhibits at least a twofold increase in biomass relative to the plant not containing the nucleic acid molecule.

5. The plant of claim 1, wherein the plant exhibits at least a twofold increase in yield relative to the plant not containing the nucleic acid molecule.

6. The plant of claim 1, wherein the plant exhibits an increase in drought tolerance relative to the plant not containing the nucleic acid molecule.

7. The plant of claim 1, wherein said plant is selected from the group consisting of Arabadopsis, canola, wheat, corn, soy, millet, sorghum, cotton, tomato, grapes, peanuts, rice, lettuce, melon, sweet potato, carrot, cucumber, sugar cane, alfalfa, Camelina or PennyCress.

8. The plant of claim 1, wherein the nucleic acid molecule further comprises a promoter, a nuclear localization signal, a coding sequence, and a termination sequence.

9. The plant of claim 1, wherein the FTO gene is expressed in a nucleus of said plant cells.

10. The plant of claim 1, wherein the FTO gene encodes a polypeptide comprising any one of SEQ ID NOs 6-30 or 39-71 or a polypeptide comprising at least 90% identity therewith.

11. A method of generating a modified plant comprising increased yield, growth rate, biomass, vigor, oil content, seed yield, fiber yield, fiber quality, fiber length, photosynthetic capacity, nitrogen use efficiency, drought tolerance and/or abiotic stress tolerance as compared to a wild type plant of the same variety, the method comprising
(a) genetically modifying a plant cell with an exogenous polynucleotide encoding a polypeptide comprising an amino acid sequence with at least 80% identity to the sequence selected from the group comprising any one of SEQ ID NOs: 6-30, and 39-71, and
(b) regenerating the modified plant from said plant cell modified with art exogenous polynucleotide.

12. The method of claim 11, wherein the method further comprises producing modified plants from the plant produced in step (b).

13. The method of claim 11, wherein the said polypeptide is from vertebrates, invertebrates, algae, or oomycetes.

14. The method of claim 11, wherein the modified plant exhibits at least a twofold increase in biomass relative to a plant not containing the nucleic acid molecule.

15. The method of claim 11, wherein the modified plant exhibits at least a twofold increase in yield relative to a plant not containing the nucleic acid molecule.

16. The method of claim 11, wherein the modified plant exhibits an increase in drought tolerance relative to a plant not containing the nucleic acid molecule.

17. The method of claim 11, wherein said plant is selected from the group consisting of Arabadopsis, canola, wheat, corn, soy, millet, sorghum, cotton, tomato, grapes, peanuts, rice, lettuce, melon, sweet potato, carrot, cucumber, sugar cane, alfalfa, Camelina or PennyCress.


SEQUENCE LISTING (71 sequences)

SEQ ID NO: 1 - protein, 505 aa, Homo sapiens
MKRTPTAEEREREAKKLRLLEELEDTWLPYLTPKDDEFYQQWQLKYPKLILREASSVSEELHKEVQEAFLTLHKHGCLFRDLVRIQGKDLLTPVSRILIGNPGCTYKYLNTRLFTVPWPVKGSNIKHTEAEIAAACETFLKLNDYLQIETIQALEELAAKEKANEDAVPLCMSADFPRVGMGSSYNGQDEVDIKSRAAYNVTLLNFMDPQKMPYLKEEPYFGMGKMAVSWHHDENLVDRSAVAVYSYSCEGPEEESEDDSHLEGRDPDIWHVGFKISWDIETPGLAIPLHQGDCYFMLDDLNATHQHCVLAGSQPRFSSTHRVAECSTGTLDYILQRCQLALQNVCDDVDNDDVSLKSFEPAVLKQGEEIHNEVEFEWLRQFWFQGNRYRKCTDWWCQPMAQLEALWKKMEGVTNAVLHEVKREGLPVEQRNEILTAILASLTARQNLRREWHARCQSRIARTLPADQKPECRPYWEKDDASMPLPFDLTDIVSELRGQLLEAKP

SEQ ID NO: 2 - protein, 475 aa, synthetic construct
MTPKDDEFYQQWQLKYPKLILREASSVSEELHKEVQEAFLTLHKHGCLFRDLVRIQGKDLLTPVSRILIGNPGCTYKYLNTRLFTVPWPVKGSNIKHTEAEIAAACETFLKLNDYLQIETIQALEELAAKEKANEDAVPLCMSADFPRVGMGSSYNGQDEVDIKSRAAYNVTLLNFMDPQKMPYLKEEPYFGMGKMAVSWHHDENLVDRSAVAVYSYSCEGPEEESEDDSHLEGRDPDIWHVGFKISWDIETPGLAIPLHQGDCYFMLDDLNATHQHCVLAGSQPQFSSTHQVAECSTGTLDYILQRCQLALQNVCDDVDNDDVSLKSFEPAVLKQGEEIHNEVEFEWLRQFWFQGNRYRKCTDWWCQPMAQLEALWKKMEGVTNAVLHEVKREGLPVEQRNEILTAILASLTARQNLRREWHARCQSRIARTLPADQKPECRPYWEKDDASMPLPFDLTDIVSELRGQLLEAKP

SEQ ID NO: 3 - protein, 419 aa, Ostreococcus lucimarinus
MSPSSSVLEPEDGEPFARVHRAHYRGFVVDAPSVLPASLHDDVERAFDDMRSRGEFTHDVVSAGNKVSTTYVRRCLLGEDGMTYHYQKLRLFAQPWRGREAYEVVRRLNETLTRSARERCEKLGGAFAESECEYNVTLINYMETEGESEIELRNEEKFDLGTTSVSWHSDSSLRENSTVAVYHTYEAPERKDWRVALRALNAECEVLCVPLEDKATYYMCGEFNATHHHAVLTGSSARYSSTHRVAVVAKDTFQYIKRRCIDALAIVPDLERENKPLDAKQIQFLADVHREVEFQWIRMFHLQGEAHAAWHDTYWTRKIAELTEAWDRMEACFRVILSKLKRSSRSPESPPRAYAMLLYALKTVKELRDEYTKRTKASAYASLPPSQRPVDLPAYDNTSPLPFELKPVIYFLEEEQNKV

SEQ ID NO: 4 - protein, 505 aa, Sus scrofa
MKRTPTAEERERGAKKLRLLEELEDTWLPYLTPKDDEFYQQWQLKYPKLILREAGSVPEGLHKEVQEAFLALHKHGCLFRDLVRIQGKDLLTPVSRLLIGNPGCTYKYLNTRLFTVPWPVKGSDAKYNEAEIGAACQTFLKLNDYLQIETIQALEELAAKEKANIDTVPACIGPDFPRVGMGSSFDGHDEVDRKSRAAYNLTLLSFMDPQKMPYLKEEPYFGMGKMAVSWHHDENLVDRSAVAVYNYSCEGPEEESEDDPQLEGRNPDVWHVGFKISWDIETPGLAIPLHQGDCYFMLDDLNATHQHCVLAGLPPRFSSTHRVAECSTGALDYILQRCQLALQNVRDEADSGEVSLKSLEPAVLKQGEEIHNEVEFEWLRQFWFQGNRYKKCTDWWCQPMTQLEELWKKMEGATHAVLREVRREGAPVEQSSDILTAILAVLTTRQNLRREWHARCQSRIARTLPVDQKPECRPYWEKDDPSMPLPFDLTDTVAELRGLLLEAKP

SEQ ID NO: 5 - protein, 505 aa, Bos taurus
MKRTPTAEEREREAKKLRLLEELEDTWLPYLTPKDDEFYQQWQLKYPKLILREAASVPELLHKEVQQAFLTLHKHGCLFRDLVRIQGKDLLTPVSRILIGNPGCTYKYLNTRLFTVPWPVKGSDAKYNEAEIAAACQTFLKLNSYLQVETIQALEELAAKEKANIDAVPVCIGPDFPRVGMGSSFDGHDEIDMKNRAAYNVTLLNFMDPQKMPYLKEEPYFGMGKMAVSWHHDENLVDRSAVAVYSYSCEGPEEESEDDPQLEGRDPDIWHVGFKISWDIETPGLAIPLHQGDCYFMLDDLNATHQHCVLAGLPPRFSSTHRVAECSTGTLEYILQRCQVALQNVREEADNGEISLKSLESVVLKQGEEIHNEVEFEWLRQFWFQGSRYKKCTDWWCQPMSQLEEMWRKMEWLTSAVLREVRREGVPMEQKNEMLTSILASITTRQNLRREWHARCQSRIARTLPADQKPECRPYWEKGDPSMPLPFDLTEIVSELRGLLLETRP

SEQ ID NO: 6 - protein, 528 aa, Acanthaster planci
MRFLSLNVLRRWMSKMSKRRQTEDLGKEVKKKKLLSEVARGRLQFLMPDHPDFKSLMRKSYFNFAHEPSTGIPVSLHEELTWALDILRYHGYFYRDWVKVKGQTVQTPISRTLIGEPGTTYKYLGLRLFTIPWSFGHHKPESEVELASETIGKLNEHYKAVSRRLLRKKTAEQTHLKSSPSQEVAPCSSKDRPRERICVEEPKQMTHGLSIEEGTNFNVVLINYMDPEDPDLRLKPEPYYGMGPLAVSWHMDGNLVRGSTVAVYNHTCSSDGGDSTTDSDWMVGFKVSWDIETPGVAIPLQTGESYFMLGDLNDTHQHCVVAGSQARFSTTHRVADCQQGTLSYIQQRCLEALGNLSQLENGEYELKSFQAGHLDFTETVHNEVEFEWLRQFWMQGSRHAAERGGWVPAMECLEADWNKMEHMTKLAVEEANRLGPEEGSVIAEALLPHLKERRKLRQECLDKFPRRLLETIHRDFLPIHRPKWDNDDPSQPLPFDLKGIIRKMRLLCNGKRIKSPSDSESSDTGLHS

SEQ ID NO: 7 - protein, 520 aa, Micromonas commoda
MGKKHKPRPDDRARVVDASDVGGKVKKKKANGATMKASRRKGGGASANGDGRPYDPSRTLSKPAPPPPAYTPRVPTGAYITPEHDEYDACVARSYVGFVVDPPDILPDELHADVAAAFKTMAKRGLFTRDVLAAGKTVSPTFVSRTLVGERGMTYHYQKLRIFALPWSDEDTPEGSPLRVVRRLNDAMKRRSRALLTRHRDARVPGDVTGSCEFNVTLINLMEPERKESVALKDEGQFGMGKASVSWHSDSSLQDTSTVAVYHQTSGGIDGDDTSWHVALKCLDGVTPALRVPLRSHSAYYIMRDFNLHHHHAVLNGDTRRFSSTHRVAVTAKDTFDYVRGRCERALAVLPSLKWRAGRWPKGTKPPRDASSTQLSDAVRVLGETHREVEFQWIRMFWLQGTRHAAQHDGYWTRRIEELTQAWDVMELGLRFAMDACARAADGKAKAEPRAFEMLRYLLETIAELREEHDARTQSGAYAQVPDDCKPVDLPGFDDSSPLPRNLRPVLNTLERHRRRALDR

SEQ ID NO: 8 - protein, 486 aa, Nitzschia inconspicua
MSPTASNERKKKRKRRKSASGSVAESSPTGTFSDHLESTTRLSYNSSIILSPSSSQIPPVLSSRSFLTSNDNGFANYVKDHYPGFVHLPVVQYEQTLQQRDFHAYARQALERLRDASYYQYDIVMAGGKHSSRTFVKRTLVGEPGITYKYLGVRLFAHAWSGPLASPAMKAIGEVNRRMIEITKRMPNPGLYNYNLTLINFMEPTSHTSIGFKEEANYGMGKVSVSWHADSSLESGSSIGVYHCFPTQRAARWDWRIALRPSPDSNKPSNIAPVVVNTKDGDLYFLLGDFNENYQHCVLAGSQANRISSTHRVAVTAEDTYDYILKRVKIARKRFRLQLEGNEDEKTFKSPSPTTLDAKIIRYCQRVLTELEMEWIAQYWLQGAEHDKMHVWWQRPMKTLEAYFSALEEYTYKLCQLCVNIDTSDMAPVDVLKVLLAEFKSRQSLRRHWDERRGDKIYQRRISAEYRPVARPLFLSKEQQDGTKLD

SEQ ID NO: 9 - protein, 512 aa, Aphanomyces cochlioides
MDAKERKKLERLKKKLKAQKKKEASASKPASTAPSVPAKPAPKNLKRPAPEHPTAPATKKQDTTTKALPVPSPYSRPEKFLTPSHPQFNEILKRSYVGFVHEPGTTLPRDFHDGMRSAFEKLRDNEYFQYDVVMAGGKHLSRTFVRRTLVGDYGITYKYLGLRIFAHAWDKPHCAPVFKSLYQLNQRMIERTKRSMKPTPPLCDYNLTLINYMEPDNPMLGLKADPTYDMGKVSVSWHADSSLQDYSSIGVYHSLLGNKKKKAPCDWRIAMRLSPEVPGAATTPPLVLPTDDGDVYYLNGDFNHFHQHMVLAGTAVRVSSTHRVAVTDEDTLQYIQGRVARALKSSANGNSPFREGEVDAVREEQATLTVVEMDWIRQYWIQGVKHDELHTDWQGPMRKLEANWLVLEERTKTLVDALLKTQDIEHLKVLKAVLEGLRARQAGRDQFVHRMRDKVFKRIHADFQPTERPTWKSGILPKDLTDVIAKLNAHKEELEKEKKMQWKANHKKQQKK

SEQ ID NO: 10 - protein, 557 aa, Chaetoceros tenuissimus
MAKKNKGKKRKNDLSKRERDGKISKEKISLPQLLKKNRGTAATTPLPPAWSTLPPNPPKFLTPDDEGFESLKSECYRGFVYENPKDIDDDIHECFSSSFHGLDQSGLFLYDVVQPGGKKLSLTFVTRTLVGNAGSTYKYLGLRLFSHPWNDVTGDGDDDLNSNNASQKTTTGESLIELGYSKKCAKALIKLGIINKKLRKRTDHVLQREIAPLVKDNLVGSADYTITLINKMEPTRSKKDLKNDKVHGLGKTSVSWHKDSGLNDFSSIAVYHQLENTSDDIPWKVALRVADANVKTPALSVPLPSKSLYYLLDDFNHQHEHAVIAGSNSLRYSSTHRVARDGAGTYQYIRDKCRKVLSYTVCKTILKAIQSNTDPSFDDSDKPKVEKELRACNQLITELEFEWIRQWYIQGKLHARLHPYWEDKISYLEECYLKLHSIVNYINSLFEENRDKYGIITEDLFDIFIEYAESRNELRLAWKQRLKDSVFKSLDVQARPSECSTLNNCKEAGGIELRQWKSNFCSKGTASKKRKREKKSALTKKEKKRVASNWEKMKKKF

SEQ ID NO: 11 - protein, 515 aa, Crotalus tigris
MRRTRAEEREKEAKKRKLLDEIGESCLPYLTPKDAGFHELWETKYSKLLLKNSEKIPRDIHKEVQTAFSTLRKHVCFFQDLVRIKGKDFVTPVSRILIGNPGCTYKYLNTRLFTVPWPTEGYDINYSNPEISLACKAFLRLNDYLQRESLLSLRDLAETPGMQNSPLGDGNQDYAPNITEMEAVLQDQNPSPVPKSDSTVLEERTSYNLTLLNYMDPQEMPSLKPEPYFGMGRMAVSWHHDENLMERSTVAVYSYSVEEDDGGALEEKYAAGRDPDVWHVGLKVAWDIETPGLAVPLHQGDCYFMLDDLNTTHQHCVLAGRQSRFSSTHRVAECSAGTLPYILDKCKAALENLSTDLNLKGPSLKSLEIGDITRVEKTHNEVEFEWLRQFWFQGKRYRRCTDWWDKPMADLEELWRQMELVTSLLLCELRGGEEEQMEEQRNEKISCLLPLLIQRQGLRQEWLLRRKCPQAKDVGKGEGLGDARRGCSADGGHLDIGKQVSLKGAEGFPPGLSTL

SEQ ID NO: 12 - protein, 555 aa, Chelonia mydas
MSHQGPQTFSRVTASQERVPHYKQKLLEELGESWLPYLTPRDAEFHQLSWFLLLPTKLECKSPEVNIGTDPICQCSIYNVASLPNVWKTRYSKLFLRKADKIPQELHHIVQEAFLTLQKHDCFFQDLVRIKEKDFLTPVSRILIGNPGCTYKYLNTRLFTVPWPMEDYEIKYCHPELFAACKALIKLNDYLNVEAVQALQEQNLSETKETENTAVTDREQDFSTFIKGSLLEVQSSLYTPRNDCTSLKYRTSYNLTLLNYMDPLQMPYLKQEPYFGMGNMAVSWHHDENLIERSTVAVYNYSCEGSACDKVNEQYVEGRDPADWHVGLKVAWDIKTPGLAIPLHPGDCYFMLDDLNMTHQHCVLAGFEPRFSSTHRVAECSTGTLEYIFGQCEFALQNLQVDSDSAALSLKSLEVQVVKEVEGIHNEVEFEWLRQFWFQGKRYRKCTDWWFEPMAKLEEFWKKMEFMTSLVLCEVGKGERTMEQRNEIISCFLPLLTERQELRKEWTARCQCQLAMSLPEEQKPKCHPLWKDDDPSMPLPYDLEEVIANLQTLLE

SEQ ID NO: 13 - protein, 493 aa, Megalops cyprinoides
MKRSGDSERKKKRKRQKLLQELGGQHIPFLSPSDPGFQQLWESSYSGLALRPAGSLPADLHDRVQTALHTLQRRGCLQRDLVRIRGRDVFTAVSRALLGRPGHTYRYLGTRLFAIPWHEEGEGAGEGACCDAELRQACRSLWELNSYFCEDVERRETAGREGEKDEDPEGGQGTSQAEPPEAAPPAPQFNITLLNYMDPAAMNDLKEEPYYGMGKMAVSWHHDENLSPQSPVAVYNYSCKDDKGDKGGSSGDSDVDGASWRVGLKVAWDIHTPGLALPLRSGDCYYMRDDLNRTHQHCVLAGETARFSSTHRVAECSTGTLEYIQRRCREALENLQADPDTGTKSLRSLLPSAIQLSEEIHNEVEFEWLRQYWFQGQRCAKFCSWWTKPMEQLEESWGQMESMTQLVLAAAEDEGSTDDRRREVAETLLNALTDRQEQRQTWRDRCQSSLAQTLPPEEAPVNRPYWGEDDPPMPLPFDLTDIISRVESLLWRL

SEQ ID NO: 14 - protein, 489 aa, Anguilla anguilla
MKRSGDSERKKKRKRQKLLQELGGQRIPFLSPSDPGFQELWESSYTGLALRPADSLPAALHDRVRAALHALQRRGCLFRDLVRIRGRDVFTAVSRSLLGGPGDTYRYLGTRLFALPWHNDAQEDGEACCDRELREACRALWELNEYFCEDVERREAAGQPEADEDPPGGQGSSKTDASEATPAAAPRFNITLLNYMDPAAMNDLKEEPYYGMGKMAVSWHHDENLVPLSSVAVYNYSCPDEKGGGSGDGEVDGASWRVGLKVAWDIHTPGLALPLQSGDCYYMRDDLNRTHQHCVLAGGTARFSSTHRVAECSTGTLDYIQRRCQEALENLRTDPDTGTKTLHSLQPSTLQLSEEIHNEVEFEWLRQYWFQGRRCARFCSWWSKPMEQLEESWAQMESMTQLALAAAEDEGSAEDRRREVAETLLTALTDRQEQRQTWRDRCQSSLAQTLPPEEAPVNRPYWGEDDPPRPLPFDLTDIISRVESLLWRL

SEQ ID NO: 15 - protein, 517 aa, Patiria miniata
MSKMSKKRQNEDQGKELKRKKLLSEVARGKLQFLMPDHPDFKSLMREKYFNFAHEPCSCIPTSLQEELPWALNILHHHGYFYRDWVKVKGQTVQTPVSRTLIGEPETTYKYLGLRLFAIPWCFGDHQVESEVELACGIIGKLNDHFKAVSRKLLLERAAEKTEPESCISQGAAQSPLHHSPNEETKSCSEQKPTTCSPSIEEGTDFNVVLINYMDPKDPDLRLKAEPYYGMGPLAVSWHMDSNLVQGSTVAVYNHTCSSDGDSSSDSSDWMVGLKVSWDIETPGVVVPLQTGESYFMLGDLNDTHQHCVVAGGQARFSTTHRVADCCQGTLPYIQRRCKEALSNLGETENGEYKLTSLAADHLELTETIHNEVEFEWLRQFWMQGSRHAVERKSWVPAMEHLEADWHKMEHMTKLAVEEANVLDDEEGSAIAEVLLPHLEERQKLRQDCLDKFSPKILETMHADFLPIHRPKWEDDDLSRPLPFDLSNIISNMRLVTKHGKKSCISDSESSDSDYHS

SEQ ID NO: 16 - protein, 490 aa, Asterias rubens
MEEGNLPPWMNIPITETEPLVGHLPSSHTRHTQPSDNPTLRAFPRRWMSNMPKRKSSGDSGKEIKKRELLSEVAKGKLPFLTPADATFESLMKEKYSNFAHEPQSCIPESLQEDLTWALNVLYYHGYFYRDWVKVKGQTVQTPISRTLVGEPGTTYKYLGLRLFTIPWSFGDVQPENEIEIACGVIGKLNEHFKVASRKLLLEKENERLERAITRTKSDEGTPSCLDKEESRGCESRDGGILSVDDGTNFNVALINYMDPTDLDVRLKPEPYYGMGSLAVSWHMDGNLVRGSTVAVYSQTYSDEDDDSIDQTDWKVGLKVSWDIETPGVAIPLASGESYFMLGNLNDTHQHCVITGSQSRFSSTHRVADSRKGTLRYIKGRCQEALCNLLEETSGDYHLRSCDALHLQLTETIHNEVEFEWLRQFWMQGTRHATERRSWLPAIEALEEEWVKMEHITKLAIDKTNRDTEESIAIARILRPFLKERQKLRK

SEQ ID NO: 17 - protein, 485 aa, Saccoglossus kowalevskii
MNPRMKRKIPRIPLVTSEGEVAGQLALPIRKQKLLSAVKETQVKFLSPVDTDYKALVAENYDNFVVQPGTELSSELHTRLQQGFETLLTNGYLYQHRIRFRNKTVMTPVSRTLIGEPGMTYRYLNLRLFALPWTFDDKKKLSDVEKACNAFYELNTYFKDHVNKLITEKFASSTGRKIDETDDSLKTIIQRNTEFKATILNYMDPSEIRLKQEPYYNMGDIAVSWHKDESLVDGSTVAVYNHVPMEARQDCSKWKVGLKVAWDITTPAVCTPLNNKDCYYMLDTMNDTHQHCVIAGTLPRYSSTHRVVESSDSTLCYIQQRCQTALDNIGYKDQIPYLKSLETDVLQLIEEIHSEIEFEWLRQFWMQGTQHSQERQYWLKPMEQLQEDWKTMELMTKLMIDIVVKKKDQSHMIAKILLPSLVERNELRNEWMNRYSEKEFYHIPEEHRPIYEPCWTDANSEMPLPFDLSQHIDIIQECMDTNDSQ

SEQ ID NO: 18 - protein, 420 aa, Ostreococcus tauri
MPDVLGPGDGDAYERVRSFHYDGFVVDDPEILPEALHDDVARAFDSLLREGEFTHDVLSAGNKVSRTFVTRTLLGDRGTTYHYQKLRLFAQPWDGEKRWEYATARRLNEALTRRTREILRGTRHGESECEYNVTLINRMDTREESEVALKDEALFDLGTTSVSWHSDSSLRENSTVAVYSTVDDPERADWSVALRALGKQCPALRVPLKDKATYYMCGDFNATHHHAVLTGSSARYSSTHRVGVVEKDTFQYIKRRCLEALALIPDLENKSASLDAKKIQLLAEIHREVEFQWIRMFHLQGDTHARQHKTYWTQRIAELTEAWDRMEACFRAILSKLKLSPESTPQPSRAYSMMLYAMKEVKELRDEHVKRISASAYAALPESQRPVDLPQYDDTSPLPFELKPVIYFVEAEQAKLKGES

SEQ ID NO: 19 - protein, 541 aa, Micromonas commoda
MATATGSSGGKLEKRKKKKKKTTAVERAGSGGGGNAKKPTTTTASHRPTKPLLRAAPPPPSYRLRGTSGSDDDDDDAAPSFLTPSHAEYERCMKTSYLGFHVDDPSALPEKTHREIARAFERMASDGIFHRDVLAAGKSVSPTFVSRTLLGERGMTYHYQRLRIFAHPWDEETQPDASHPFRAVKRLNDALVSRSTAVLRASSDPRLGDGVDGSCDFNVTLINLMEPASRCAEVPLKDEALFGMGKARSTSVSWHSDSSLQDVSTVAVYHQTDDDDDDSWAVALKTLDGVTPAMRTPLRSREMYYMLRDFNAHHHHAVITGESRRFSSTHRVAVVAKDTFEHIKTRCERALRLLPGIKKIAQVGPGPGDDLAAFARSAQMMGETHRECEFQWIRMFWLQGSRHAASHSAYWRQRIAELTQAWDAMELGYRWALDALRAGASTGAMPLRAYDVVSYLLEETRELRAEHARRSRAGAYEAMPADCRPVDEPKFDEKSPLPFDLGPVLATLAGWRRDAERAQTTTTEGGSKTKKGKPGKGSKRR

SEQ ID NO: 20 - protein, 715 aa, Ectocarpus siliculosus
MTTTSATTAARSRLPSPPTSAATTPIQHSNNGTEASPEKTSNKKKAKNKRKRQQECSIPAASQQSRGSSSSSTRPPPPTTTTLKKHSRIKEKPARFVHPHLDAELRRLGRSWLSPGDPLFTPALDRSYRGVRHEPAGSLPQAVHSGFRQSFDSLHEAGLFLYDTVQAGGKRLSRTFVTRTLVGDAGITYKYLGLRLFAHPWGLGESDFRKGPGDGLADALNDPCLADLFSKDGASSVRKSKDNLRGSRFDDNDRVADTLRKVGDLSEWMRLRAGAMLKEEVREAGGERGSCAFNLTLINRMECSGAKRDLKPDPLFSMGKCSVSWHADSCLQDFSTIGVYHCTDPVPAEPDWGIALRVSTEARVGGDGGGDGDGNGVSNDEKTRRKEAERGLKPAGPKAVASVAASAAEAVPEKQGEQVLTPPLLIPLQSGDAYFLLDDFNHFHEHAVVSGSSTLRYSSTHRVADTDGNTFSHVQKRCEQALAGGGSAAAGGDDQILNLAQVRAEQALLTELESEWLNQFWVQGRRHASLHPWWRGPMALVLATWRELERRTSLTVQHLMSLRGQDGGEALWPAEGSGGGDVGEEGSTLLLPQSLGGVISRLRECYTVVYRAASGGKKAGSAKAIAAGAASNWELFRAASSSASSGGGGAGKKHGSALGKQQQQQQQRKKRGAAGQDGLEKKKRAGGVVKGGGDGGGGRTSKGQRSRRKGRVRER

SEQ ID NO: 21 - protein, 1065 aa, Ectocarpus siliculosus
MAKSSGDRGRGGRRGRGRGTLSKGKGGRGSGSSSQQRYQQTQQHHPARISVKPPSFLGFRGPSDRFVEESHPQFQEIVSKCFAGFHVEAAEAFDAKVHEEARQAFDGLEEGGVFRADVTQPFGLGTKCAVTYVTRTLAGDPGTTYKYLGLRMFSHPWTTTAKEADSDGGHGSGVPSALRRVGLLNNKLKERSLRLLRARGGRKGKARREGGEGNEFGSCEFNVALINRMEPSDDRPDLKLEPTFGQDRCSVSWHADSCLEHYSSIAVYHVTDPEAKNKNADADGGDGSPAVPADWRIALRVEPDAEGPSAGKLKLAATEDKSSRVDRAPAVAVALPSRSAYFLLDDFNHHHQHAVLAGQSHRWSSTHRVAKKQGHSFDYISGRCRTVLGDGHRRTLKQWKTEQLTLAELEFEWIRQFYVQGRAHHALHSWWHGPLQELLRFWSLLEAKTAVVVNTLQGAAESRFDHHQQQQSGGSVVEAAGAGADDKREKKARQKRAKQRQAVESLGGRDAYSAIADALGDRAEKRRQWAEREKDKALSALADAVLEWGKIFSGEDGVRGGGIVVAEEAGVGGGGRRVSGGCGENESGGTGEKPAGVADSGAGGVDNGPDGQEPCESSTSGGSSLPSPLVTSPAGTESARHAPPTPWETGRFALEMQAPWSRRLLDGEKTVETRGYPLPEGLVGRSIEVLESGRGRDGVSGVGDVVEAFSAGLSVVGRVVFGGTEAYPSREAWAADAERHLVPVPAEEEGSESGKGYGWKGPGSVHAWRVASVEAYPKPRAVRRMGRVFRSLFLVEQPKGKQPTGGGGGGGGGGGGNGANPDGSVGVGAADGRPEIGGDEGKRKRKPKKKRKRKGPPAGEVLGAADAAPGTAVSEKGNPLSASSAAVETPNATAAAAPTAVSPTGGAKGASAEKPKTKRPRHAANGQQGTVSKEGNVFDGPASGGEESLSNGSGSVQDGGGVGGDGGGGGIGVEDSKESGCGEKGTRRRAGKGSEQQKGLTRKGGEEEVGDEGVVALAAAAAAARSAGGGFDVVVGDGWGAVDAPAVKPGDGEMRKRHKRKKKRY

SEQ ID NO: 22 - protein, 546 aa, Thalassiosira pseudonana
MAKKRKSDKNTWQKDGLPPEKKSGKRKREKEGQLQLLPSAASTDAVKNKASILKKHRGVAANTPAPPIWKDTKPTPRAFLTPQEDPKAFDSCLKTSYTGFRYDSPSALPSTLHGEFESSFDGMDAGDLFLYDTVQPGKKRLTRTSVTRTLVGDPGSTYKYLGLRLFSHPWTDVDKDGNMQSKEQKRVGSTLHKLGYSEKTTSALLAMGRANAYLTERSNTMLKKYVAPNVQPPGLVGSAEYNLTLVNKMASTTMKRDLKNEMAYNMGKISVGWHRDSGLKDFSSIAVYQTLKDTATANSWGVAIRAMDGGTGGPLASVPALLVPLPSGSLYYMFDDFNHNHEHAVIAGSDGVRYSSTHRVAREGAGTWQYIRDKTKQVLDASTKFNSSEKTCQASKKTFEKFVAHVRLQQLLMNEVEFEWLRQWYVQGSKHASLHPYWHQPIEVLCDSYSNMEQRSVDLLVRMKRASHDNKTETFNTELSEDLFDVFIETLTERSNQRSGWTARYRDPIFGEIPADERPFPCPLLDRTGDMTLDESLKKGQMPEDL

SEQ ID NO: 23 - protein, 939 aa, Polarella glacialis
MSLSLQLVPLQAGLGGPWLLPPVGSAPLTIGRKTSCELLMPEAHAYVSGEHCRVRSVVQESKFCVDLEDLSGNGTYVNGGKVGKGNHRNVCTGDEISLAKPSRRGGAIMFRLEVVSAAVSAAVPAAVPAAPPVAHPVVPAVSATKQATPLEGRSASGDLAAALAVSARQRQGENHRLQALFEAEKVRCASLQAELLEERRQLAEERRRRQESQAAKVLVHAQAAASRAGSSDALLKNNARKQGDCEELRESLRRISAEHAPLERAQLSAEEAAKRESRECVRLRAELQEELRCAERAALEKDQLKRQAEEAIGRQLELEAEVRREAAFNASLEDVKSPAVCSGFGQLVQLLSFRFMAAFQKGLGMGQVSGCNCFDQEALGAQPPKFRLISKWCQFHRPKFRRHYAWRQNFKINTSTDTMVMQLMPLWNKKHRLYESLGFPSGRLLSAAMDMDRFVREPADASSESFHEEFRRCLHGLYEQGIFQYDLIQPVGPGTPVVSTRVTRCLIGEPGITYKYLGLRMFAHPWKGEKTSESCSAMYQHNQELISKAEEQAAAVGCDAPPYSSCFNLTLINRMDPADSGAPNKLGAVSVSWHADSSLVDYSTITVYVAEHDATQDSFGLSPAKPSKPCDDPWRVALRHTPNVQGPDKSCPWEVAAQETAKPAKRRAKAKVHRVPIHDGDIYHLIGDFNHHNQHAVLAGSDAVLAVRRGTPVLGGSGTIRYSSTHRVALTEGHSYRSVKQRCSTALSVALADMIPAAAQVHEEQQCSLELEFEWIRQFFIQGKAHREALPWWHSKMDELVFLWRQFEVLTAQRLTLLRRAAAGKVGTSELDPFCLALKNGLQKRAELREAWRQREHDPLLKKQPANRQPLQCLFDAPSDAPHGAGSEALPHNVWPLVEEISQIQLQLHETKKVGKPWYQFVLNVYQGCAFQGGEVTLV

SEQ ID NO: 24 - protein, 564 aa, Pseudo-nitzschia multistriata
MASKSSKGGGRKRRRESGRDNCDASTQPRPATKTTNTATKTTTTTTTTTTTTSYSSSSLPRFLTPSDPNFEKHVAEHYKGFAHCSVDDLRPEGFHDRARDTLERLRDANYYQYDVVMAGGKHSSRTFVRRTLVGNPGITYKYLGLRLFAHAWSGPGASPLMQSIGEMNRDMIRMTRRFEDHQHCEYNLTLINYMEPTIHTRVGFKEEADYGMGKVSVSWHADSSLEPGSSIGVYHCLPTQRSSRWDWRIALRPSPDGGSTARPIVVDTKDGDAYFLLGSFNETHQHCVLAGSQSNRISSTHRVAVTAEDTYEYILKRAKIARKRFRGQLEAIDGSSDSSDPAAEVDAKVVRYCQRVMTELESEWIAQYWLQGIQHDIMHTWWQKPMKTLEAYWRALELATARLVRRVCSREPSAKKIPRDVARVLVHEFKARQALREQWDDRRADKIYQRRISEEYRPVARPVFDAPDPSNNNDDDALLPKDLSPVLMDLRLVLEESAAGLAVQTPPVAKSEATPKPDKAAPKARANDGEAKHSNTKKRRKKNKKKKGSMNTPTDAGKSSTKPF

SEQ ID NO: 25 - protein, 1060 aa, Tribonema minus
MHSLVREAFSKVHEAGLFLYDIVQAGGKRLSSTFVTRTLVGEAGITYKYLGLRIFAHPWCGGGQADVGQQQQQQQHQRKKKHKGESGAAAAAAVVAAAEDAAVTELWGQGEVESLGPKVVTALRQLCTLNAWLRRRAERACAEEGITARLAAAGHSPGSFDFNLTLINRMEPSGHGDGYHAKLKPEPIFGMGKASVSWHADSCLEDYSTIGVYHVTTPPVTTSMTTSKNRSSSSGGGGGADWKAVASAKAEGEEVVTPPLVVPLRSGDAYFMLDDFNHYHEHLVLAGEDTLRYSSTHRVSVREGNTWAQISGRCREVLSVSAPTAPALPLAQLRAEQALQTELEGEWLYQFWAQGRRHARLHPWWRRPMNALVRWWRELERRSHAAVTHLLSPQSEGAELPRAVETLARCLEARRQQRARWAERLADAAYRALPPDAAPLRPGLWGGGGGGGGGSGGGSAALPYDLTGTVAKLRERYAAAHKAATGGTAVATSARALVAGATSNWAALQQGKGGLAAALAGAAAHRAQQQAPQTQNGGGPAAKQGKPKGLKLKNGVGVNSGNGYASGTSGGGRGGDGSGAGPDSGGAVMRGRRTANKPAVKKRAPKRKKDTSSSSPAVVYHATPGQMPPSVVPNMLDLLSVMDLCGLGRVSRAWRTESQRECHYARRATPAALDPYAFYAQDEETGERTEEVPFTELGEARLAGLIEACGAWRVVVALASRRCVGCGARTGWFSYCACARTCAACFRAPAAKLGSLSYAKVRARVVCACARGSDVGTYVMRFLRCVACGVRTGWISYCACARTCAACFRAPTAKLCSVSYTKLCSVSYAKAHFLLSASQEAELVTLVVPEAAESGCVANRKASLPITHLGRARDSAIRRFGSEEGIMHLSRASAIDRFGSEEAFDAEMTRRRSKAQDGHDARYRAAQVATTEARARGDASAVIKSPSSPRILGLANETNYVGANQASGHVYGAKYGHVFVSDDADEVRERLSELPPRLSQSAAVPPALRPAAYYLDMQPLHVLGSAAVPPALRPAAHYLDAPPLHVLGILFGMEIVHGDY

SEQ ID NO: 26 - protein, 512 aa, Aphanomyces eutreiches
MDAKERKKLERLKKKLKAQKKKEASAPKPASTAPSVPAKPAPKSLKRPAPEHPTAPATKKQDTTTKAFPVPSPYSGPEKFLTPSHPQFNEILKRSYVGFVHEPETTLPRDFHDGMRSAFEKLRDNEYFQYDVVMAGGKHLSRTFVRRTLVGDYGITYKYLGLRIFAHAWDKPHCAPVFKSLYQLNQRMIERTKRSMKPTPPLCDYNLTLINYMEPDNPMLGLKADPTYDMGKVSVSWHADSSLQDYSSIGVYHSLLGNKKKKAPCDWRIAMRLSPEVPGAATTPPLVLPTDDGDVYYLNGDFNHFHQHMVLAGTAVRVSSTHRVAVTDEDTLQYIQGRVARALKSSANGNSPFRDGEVDAVREEQATLTVVEMDWIRQYWIQGVKHDELHTDWQGPMRKLEASWLVLEERTKTLVDALLKTQDIEHLKVLKAVLEGLRARQAGRDQFVHRMRDKVFKRIHADFQPTERLTWKSGILPKDLTDVIAKLNAHKEELEKEKKKQWKANRKKQQKK

SEQ ID NO: 27 - protein, 522 aa, Aphanomyces astaci
MDPKERKKLERQKKKAKALERKSAKKPNVASLKPVSQPTTSHKRPSEPSGSSAAPPAKKQQIEKAKPLPVPSNYTGPEKFLTPSHPDFHDILTRSYVGFVHEPAAAQPREFHAGMRAAFEHLRDKGYFQYDVVMAGGKHLSRTFVRRTLVGNFGITYKYLGLRIFAHAWDVPSSAAVFRNLYALNQAIVARTKASASPAPPSCEYNLTLINYMEPDNPVLGLKDDLTYDMGKVSVSWHADSSLEDFSSIGVYHTLLPTKKSKKGPCDWRIAMRLSPDVPGAKTTPPLVVPTGDGDVYYLNGDFNHHHQHMVLSGTAVRVSSTHRVAVTAEDTLQYIQAKVTAALKSSAKDPQDKCRRGHADDVRHEQTTVTTLEMDWLRQYWIQGDKHNDLHVDWQQPMLALEASWRKLEARTKTLADALVQSHDVDDLKVLKAVLEGLRNRQVGRDQFVHRMKDKVFKRIAADFQPMERPVWTDWDDVHLLPKDLTATIAALHAHKLVLESEKKRQWKIHAGTRHNKINKA

SEQ ID NO: 28 - protein, 539 aa, Aphanomyces invadans
MDPKERKKLERQKKKAKAQQRKSSKLSNAIPPMPVAKQATNAGNSTSMSQKRPSENAPQCAAAPPTKKQQVKQAKALPIASNYTGPEKFLTPSHPSFHDILTRSYVGFVHEPAAKHPRGFHTGMRTAFETLRDKGYFQYDVVMAGGKHLSRTFVRRTLVGNYGITYKYLGLRIFAHAWDRPNSAPVFRNIHALNAAFIARTKASMTPPPPSCEYNLTLINYMEPDNPLLGLKEDPTYGMGKVSVSWHADSSLEDYSSIGVYHTLLPSKPAKKGTKVGVCDWRIAMRLSPDVPGAKTTPPLVLPTGDGDVYYLNGDFNHFHQHMVLSGTAVRVSSTHRVAVTAEDTLQFIQAKVSAALQSSTVQSVPNDADVACRRGHADAVRAEQATITTLEMEWIRQYWIQGAKHNDLHVDWQHPMAALEASWRKLEARTKTLVGALLATQHVEDLKVVKAVLEGLRGRQAGRDQFVHRMRDKVFKRIDPDFQPMERPVWSDWDDRHLLPKDLTSTIHALHSHKHVLEAEKKRQWKDHAKQHKKQLHA

SEQ ID NO: 29 - protein, 516 aa, Aphanomyces stellatus
MDAKERKKLERQKKKIKAQQKKKAAPPPPKPTPPPSKGKAAPPASGVKRPAADAEHLSAPPAKKKDVAPKAMPLPSPYTGSEKYLTPAHPMFDEILARSFVGFVHENAAKLPKDLHAGMRLAFEKLRDKGYFQYDVVMAGGKHLSRTFVRRTLVGDFGITYKYLGLRIFAHAWNRPSSAPVFQSLYDLNQQMIARTKRSMVPAPPLCEYNLTLINYMEPDNPLLGLKDDPTYGMGKVSVSWHADSSLQDYSSIGVYHTLLPTKQKQPKTRCDWRIALRLSPEVPGADQTPPLVLPTDDGDAYYLNGDFNHFHQHMVLSGTAVRVSSTHRVAVTDEDTLQYIQARVKTAVVSSESKTPFRLDEAEHVRVEQATITTVEMDWIRQYWIQGAKHDELHVDWQAPMQKLEGQWRLLEGRTKTLVDALLETHDTAHLKVLKAVLEGLRARQAGRDQFVHRMRDKVFKRIHADFQPMERPTWDETRRLLPKDLTDTIATLNTHKQALEKEKKKQWKHAKANK

SEQ ID NO: 30 - protein, 577 aa, Mayamaea pseudoterrestris
MGKNRGKRDRKTVKEESSDDDLLLAAAAAAFPPKQQQSTTTTTKKQHHAFKDIVPPPFPLPIPSSLKGFLSNTDNQHKHLYEAAMQTCYEGVTWEENVFSETRVQQALQTLDDAGFFRVDVTQPAGLGAKCAPTFVTRCLVGKRGMTYKYLGLRMFAHPWDGGAPGCYSEPVAHALRVISSLNDTLTERTKVHLTSLDARKRARGEPGTKGRPGFDVSLINKMASMPRLKDEPMYKQAKCTVSWHADSSLEHYSTIAVYQTIRQNLNKSQSRDNHEWSVALRVAHNAEGPKAARLGSIVVDTKSPPIKLSLPTASVYYLVDDANHHLQHAVLAPKVVGNISGTRYSSTHRLLREGAQVSSMISRCEAICQKFAHHGPKRWRIEQLLLNEIESEWLRQFFIQGQAHKDLLWGFWCEPISLLFKYWSKLEMRTFQVAVEEVYRTSTGIYESFSELLADRARNRGLWVERERDPSFKRVDDALKPLEFPAEYNVRNGKIKSEGQFLGVSPFPDGSNDFLEHIAEMVRQWGEAFANATADSLPSEDPVKVAGGKLIDLNFDLVPLVQSKKKKGKKRKASLT

SEQ ID NO: 31 - DNA, 1428 bp, Homo sapiens
  source 1..1428: mol_type = other DNA
atgacccccaaagatgatgaattctatcagcagtggcagctgaaatatcctaaactaattctccgagaagccagcagtgtatctgaggagctccataaagaggttcaagaagcctttctcacactgcacaagcatggctgcttatttcgggacctggttaggatccaaggcaaagatctgctcactccggtatctcgcatcctcattggtaatccaggctgcacctacaagtacctgaacaccaggctctttacggtcccctggccagtgaaagggtctaatataaaacacaccgaggctgaaatagccgctgcttgtgagaccttcctcaagctcaatgactacctgcagatagaaaccatccaggctttggaagaacttgctgccaaagagaaggctaatgaggatgctgtgccattgtgtatgtctgcagatttccccagggttgggatgggttcatcctacaacggacaagatgaagtggacattaagagcagagcagcatacaacgtaactttgctgaatttcatggatcctcagaaaatgccatacctgaaagaggaaccttattttggcatggggaaaatggcagtgagctggcatcatgatgaaaatctggtggacaggtcagcggtggcagtgtacagttatagctgtgaaggccctgaagaggaaagtgaggatgactctcatctcgaaggcagggatcctgatatttggcatgttggttttaagatctcatgggacatagagacacctggtttggcgataccccttcaccaaggagactgctatttcatgcttgatgatctcaatgccacccaccaacactgtgttttggccggttcacaacctcggtttagttccacccaccgagtggcagagtgctcaacaggaaccttggattatattttacaacgctgtcagttggctctgcagaatgtctgtgacgatgtggacaatgatgatgtctctttgaaatcctttgagcctgcagttttgaaacaaggagaagaaattcataatgaggtcgagtttgagtggctgaggcagttttggtttcaaggcaatcgatacagaaagtgcactgactggtggtgtcaacccatggctcaactggaagcactgtggaagaagatggagggtgtgacaaatgctgtgcttcatgaagttaaaagagaggggctccccgtggaacaaaggaatgaaatcttgactgccatccttgcctcgctcactgcacgccagaacctgaggagagaatggcatgccaggtgccagtcacgaattgcccgaacattacctgctgatcagaagccagaatgtcggccatactgggaaaaggatgatgcttcgatgcctctgccgtttgacctcacagacatcgtttcagaactcagaggtcagcttctggaagcaaaaccctaa

SEQ ID NO: 32 - DNA, 1428 bp, synthetic construct
  source 1..1428: mol_type = other DNA
atgacccccaaagatgatgaattctatcagcagtggcagctgaaatatcctaaactaattctccgagaagccagcagtgtatctgaggagctccataaagaggttcaagaagcctttctcacactgcacaagcatggctgcttatttcgggacctggttaggatccaaggcaaagatctgctcactccggtatctcgcatcctcattggtaatccaggctgcacctacaagtacctgaacaccaggctctttacggtcccctggccagtgaaagggtctaatataaaacacaccgaggctgaaatagccgctgcttgtgagaccttcctcaagctcaatgactacctgcagatagaaaccatccaggctttggaagaacttgctgccaaagagaaggctaatgaggatgctgtgccattgtgtatgtctgcagatttccccagggttgggatgggttcatcctacaacggacaagatgaagtggacattaagagcagagcagcatacaacgtaactttgctgaatttcatggatcctcagaaaatgccatacctgaaagaggaaccttattttggcatggggaaaatggcagtgagctggcatcatgatgaaaatctggtggacaggtcagcggtggcagtgtacagttatagctgtgaaggccctgaagaggaaagtgaggatgactctcatctcgaaggcagggatcctgatatttggcatgttggttttaagatctcatgggacatagagacacctggtttggcgataccccttcaccaaggagactgctatttcatgcttgatgatctcaatgccacccaccaacactgtgttttggccggttcacaacctcagtttagttccacccaccaagtggcagagtgctcaacaggaaccttggattatattttacaacgctgtcagttggctctgcagaatgtctgtgacgatgtggacaatgatgatgtctctttgaaatcctttgagcctgcagttttgaaacaaggagaagaaattcataatgaggtcgagtttgagtggctgaggcagttttggtttcaaggcaatcgatacagaaagtgcactgactggtggtgtcaacccatggctcaactggaagcactgtggaagaagatggagggtgtgacaaatgctgtgcttcatgaagttaaaagagaggggctccccgtggaacaaaggaatgaaatcttgactgccatccttgcctcgctcactgcacgccagaacctgaggagagaatggcatgccaggtgccagtcacgaattgcccgaacattacctgctgatcagaagccagaatgtcggccatactgggaaaaggatgatgcttcgatgcctctgccgtttgacctcacagacatcgtttcagaactcagaggtcagcttctggaagcaaaaccctaa

SEQ ID NO: 33 - DNA, 1263 bp, Ostreococcus lucimarinus
  source 1..1263: mol_type = other DNA
atgatgtcgccgtcatcctccgtgctcgaacccgaggacggcgaaccgttcgcgcgcgtccatcgagcgcactaccgcggtttcgtcgtggacgcgccctcggtgcttcccgcgtcgcttcacgacgacgtcgaacgcgcgttcgacgacatgcgcagccgaggcgaattcacgcacgacgtcgtttccgccggaaataaagtgtccacgacgtacgtgcggcgctgcctgctcggtgaggacggaatgacgtatcattatcagaagttacgcctgtttgcgcaaccgtggcgcggacgggaggcgtacgaagtggtgagacggttgaacgagacgctgacgaggagcgcgcgagagcgatgcgaaaagctcggcggagcgttcgcggagagtgagtgcgaatacaacgtgacgctgattaattacatggaaacagagggtgagagcgaaatagagctacggaatgaggagaagttcgatctcgggacgacgtcggtgagttggcacagcgattcgtcgttgcgagaaaactccaccgtggcggtgtatcacacatacgaggcgccagagagaaaagactggcgagtggcgctgagggcgttgaacgcggagtgcgaggtgctttgcgtgcctttggaggataaagcgacgtattacatgtgtggtgagttcaacgccactcaccatcacgccgtgctcaccggctcgagtgcgagatattcttcgacacatcgagtcgccgtggtggccaaggatacgttccagtacatcaaaaggcggtgtatcgatgcgctcgcaattgtaccagacttagagcgcgagaataaacctttggatgcgaaacagatacagttcttagccgacgtccatcgcgaggttgaatttcaatggattagaatgtttcacctgcaaggcgaagcacacgcggcgtggcacgatacgtattggacgagaaaaatcgccgagctcaccgaggcgtgggatcgcatggaggcttgctttcgagtgattttgtccaagctcaagcgttcatctcgctcgcccgagtcgccgcctcgagcgtacgcgatgctgctttacgcgctgaaaaccgtcaaggagttgcgagacgagtacacgaagcgaaccaaggcgtcggcgtacgcgtcgctaccgccgtcgcaacgtccagttgatctgcccgcgtacgacaatacttctccccttccgttcgagctcaaacccgtgatttactttctcgaggaggagcaaaacaaagtgtaa

SEQ ID NO: 34 - DNA, 1425 bp, Micromonas commoda
  source 1..1425: mol_type = other DNA
atgggcagcagccatcatcatcatcatcacagcagcggcctggtgccgcgcggcagccatatgccggcgtacaccccacgcgtccccaccggcgcgtacatcacccccgagcacgacgagtacgacgcgtgcgtggcgcgatcgtacgtcgggttcgtggtcgatccccccgacattttgcccgacgaactgcacgccgacgtcgccgccgcgtttaagacgatggcgaagcggggtctcttcacgcgcgacgtcctcgccgcgggaaagacggtgtcccccacgttcgtgtcgcggaccctcgtcggggagcgcgggatgacctaccactaccaaaagcttcgcatcttcgccttgccgtggtccgacgaggacacaccggaggggtcgccgctgcgcgtggttcgcagactcaacgacgcgatgaagcgccggtcaagggcgctgttgacgcgacatcgcgatgcgcgcgtccccggggacgtcaccggctcgtgcgagttcaacgtgacgctcatcaacttgatggaacccgagcgaaaggaatccgtcgcgctcaaggacgaggggcagttcgggatgggcaaggcttccgtgtcgtggcactcggactcctccctgcaggacacgagcacggtggcggtgtaccaccagacgtcgggcggcatcgacggcgacgacacgtcctggcacgtcgcgctcaagtgcctcgacggggtcaccccggcgctgcgggtgccgcttcgttcgcactccgcgtactacattatgcgagactttaacctccaccaccaccacgcggtgctcaacggcgacacccgccggttcagctccacgcatcgcgtcgccgtcaccgcgaaagacaccttcgattacgtcagaggtcggtgcgagcgcgcgctcgccgtgctgccctcgctgaaatggcgagcgggtagatggccgaaagggaccaaaccaccgagggacgcgagtagtacacagctgtccgacgcggtgcgggtgcttggggagacgcaccgcgaggtggagtttcagtggatacggatgttctggctgcagggcacgaggcacgcggcgcaacacgacgggtactggacgcgacggatcgaggagctgacgcaggcgtgggacgtgatggagctcggtttgcgtttcgcgatggatgcgtgcgcgagagccgcggatggaaaggcgaaggcggagcccagagctttcgagatgcttcggtaccttctggagacaatcgcggagctcagggaggagcacgacgctcgaacgcagtccggggcgtacgcgcaggtgcccgacgattgcaagccggtggacctgcccggctttgacgactcgtcgccgctgccgcggaacctgaggccggtgctcaacaccctcgagcgccatcgacggagggcgctcgaccgataa

SEQ ID NO: 35 - DNA, 1416 bp, Acanthaster planci
  source 1..1416: mol_type = other DNA
atgcccgatcatcctgacttcaagtcactgatgagaaaaagttacttcaactttgcacatgagccaagcaccggtattccagtatccttacatgaagaactgacatgggcattggacatacttcgttaccatggttacttctaccgggactgggtgaaagtcaaaggtcagactgttcagacgcccatctcacgtactctgattggtgagccagggacaacgtacaagtacctgggcttgcgcctgtttacaatcccatggtcctttgggcaccataagcctgaaagtgaggttgaacttgcaagtgagactattggaaagctcaacgagcattacaaggctgtctccaggagattactgcgcaagaaaacagcagaacagactcatttgaagagttctccctcacaagaagttgcaccctgctcctcaaaggacaggccacgagagaggatctgtgttgaagaaccaaagcaaatgacccatgggctgtccatcgaagaaggaaccaatttcaatgtggtgctcatcaattacatggacccagaggacccagatttgaggctgaagccggagccttattatgggatgggacccctggctgtcagctggcacatggatggtaacctcgttcggggctccacagtggcagtgtacaaccacacatgctcatcagatggaggagattcgaccacagactccgattggatggttggcttcaaggtatcatgggacattgagactccaggtgtcgccattcccctacaaactggggagtcctacttcatgttgggagatctgaatgacacacaccagcactgtgtggtggctggcagccaggctaggttcagcacaactcacagggttgctgattgccagcaggggactctctcatacatccagcagcgatgcctggaggctctaggtaacctaagtcagttggagaatggcgaatatgaattgaaatcatttcaagctggtcatcttgatttcactgaaactgttcacaatgaggtggagtttgaatggctgcgtcagttctggatgcaaggatcaaggcacgctgcagagagggggggctgggtaccagctatggagtgcctggaggcagactggaataagatggaacatatgacaaaacttgcagttgaagaagcaaatcgtcttggccctgaggaaggctccgtgattgctgaggccctcctcccacatttgaaggaaagacggaagttgcgacaggagtgtctcgacaagtttccacgtaggctcttagagaccattcatcgtgacttcctacccatccacaggccaaagtgggacaatgatgacccctctcaacctttgccctttgacctcaaaggcatcatccggaagatgagactgttgtgcaatggaaagcgaatcaagtctcccagttaa

SEQ ID NO: 36 - DNA, 1419 bp, Chaetoceros tenuissimus
  source 1..1419: mol_type = other DNA
atgacaccggatgatgaaggttttgaaagtctaaagagcgaatgttacaggggttttgtgtatgaaaatcctaaagatattgatgacgatatacatgaatgcttctcatcttcctttcatggtttggaccaatctggattatttctttatgacgtagttcaacccggcggtaaaaaacttagtctaacatttgtaacaagaactttggttggaaatgcagggtccacgtataagtaccttggtttgcgacttttttcccatccttggaatgatgttactggagatggtgatgatgatctgaatagtaataatgcatcacaaaagactaccacaggagaatcacttatcgagctggggtattcaaagaaatgcgcaaaagctctgatcaaattaggtataataaacaaaaaactgagaaagagaacagatcatgtgcttcaaagggagatagcaccacttgtgaaagataatttggtgggttcagcagattacacaattactttgatcaacaaaatggaacctacacgatccaaaaaagatttaaagaatgacaaggtgcatggtttgggaaaaacgagtgtatcttggcacaaggacagtggtctgaatgatttctcttcaattgctgtttatcaccaacttgagaatacaagtgatgacatcccatggaaagttgctttaagagttgcagatgcaaacgttaaaacaccagcattatcagtaccactcccaagtaaatctctatactacctcttggatgatttcaaccaccagcatgaacatgctgtgattgctggatctaattcactccgatattcatcaacccatcgtgtcgctagagatggagcaggaacatatcaatacattagagataaatgtcgcaaagtactatcatatactgtgtgcaagactattttgaaggcaatacagtcaaatacagacccttcctttgacgatagtgacaagcccaaggtggaaaaagagttgagagcatgcaatcagttgataacagaattagaatttgagtggattcgtcagtggtacattcaaggcaaattgcacgctcgattacatccctattgggaggataaaatatcttatcttgaagagtgctatttgaagcttcacagcattgtaaattatatcaatagcctattcgaagagaatagagataaatatggtatcataacagaagacctatttgacattttcattgagtatgccgaaagtagaaatgaattgaggcttgcgtggaaacaaaggcttaaagattctgtattcaagtctcttgacgtgcaagcacgaccttccgaatgctcaactttgaataactgtaaggaagctgggggaatagaattgagacaatggaaaagtaatttttgcagtaagggtactgccagcaaaaagaggaaacgataa

SEQ ID NO: 37 - DNA, 1359 bp, Nitzschia inconspicua
  source 1..1359: mol_type = other DNA
atgacttcaaacgataatggatttgcaaattacgtcaaagaccactaccccggctttgtacatttacctgtagttcaatacgaacagacgttgcaacaacgcgattttcatgcctacgccagacaagcgttggagcgtctgcgagatgccagctactatcaatacgacattgtcatggcgggaggcaaacattcatcgcggacatttgtcaagagaacacttgtaggagaacccggtattacctacaaatatttgggagtacgattgttcgctcatgcctggtcaggacctttggcatcacctgccatgaaagccattggagaagtgaatagaagaatgattgaaatcaccaaacgaatgcccaatcctggattatacaactacaacttgacacttatcaacttcatggagccgacgagtcatacatccattggattcaaggaagaagcaaactatggaatgggaaaagtcagtgtcagctggcacgctgattcatcgttagaaagtggaagttctatcggcgtgtatcattgctttccaacacaacgtgctgcccgttgggattggagaattgctttgcgaccttccccggactcgaacaaacccagcaatattgccccagttgtggtgaataccaaagatggagatttgtactttttgctgggagatttcaatgaaaactatcaacattgtgtacttgcaggaagtcaagccaatcgcattagtagtactcatcgcgttgctgtaacagcagaagacacctatgactatattttgaaacgagtcaagattgctcgaaaacgatttcgtttacaactcgagggaaatgaagacgagaagacattcaaatctccatctccgacaacgctcgatgccaagattattcgatattgccaacgagtgctgaccgaactggaaatggagtggattgctcaatattggttgcaaggggcagaacatgacaaaatgcacgtgtggtggcaacgacccatgaaaacgctcgaagcatattttagtgcgttggaggagtacacctataaactttgtcaattgtgtgtaaatatcgatacctcggatatggctcctgtcgatgttttaaaggtactgcttgcggaatttaaatcacgacaatcccttcggcggcattgggacgagcgacgtggcgacaagatttatcagcgtcgcatttccgctgaatacagacctgtagctcggccattgtttctctcaaaggaacaacaagacggtacaaaacttgatgaacgactacctaaagacttgtctagcactattcaccagctgtcgcaagttctaaagaaacgatctggaaatgttcataccaacgaacagaacggataa

SEQ ID NO: 38 - DNA, 1299 bp, Aphanomyces cochlioides
  source 1..1299: mol_type = other DNA
atgacgccttcgcatcctcagtttaacgagatcctgaagcgctcgtacgttggctttgtccatgaacccggaacaactctacctcgagatttccacgacggaatgcgctctgcctttgaaaagctccgagacaatgagtactttcagtacgatgtggtcatggctggaggaaagcacctctcgcggacgtttgttcgccgaacactagttggagactacggcatcacgtacaaatacctcggtcttcgcatatttgctcacgcttgggacaagccccactgtgctccagtctttaagagcctctaccagctcaatcaacgcatgatcgagcgtaccaagcgatcgatgaaaccaacgccgccactatgcgactacaatttgacactgatcaactacatggagcctgataatccgatgctcgggctcaaggccgatccaacgtatgacatgggcaaagtcagcgtgagttggcacgccgactcgtctcttcaagactactcttcaattggagtctaccactcgctcttgggcaacaagaagaagaaagctccctgtgactggcgtatcgccatgcgtctttcgcctgaagtgcctggagctgccacgacgccgccacttgtgctaccgaccgatgatggagacgtctactaccttaatggcgacttcaaccatttccaccagcacatggtgctagctggcactgctgtgcgcgtgagcagcacccatcgagtagctgtcacggacgaagacacactgcagtacatacaaggccgcgttgctcgagctttaaagagtagtgcgaatggaaactcccccttccgtgagggagaagttgatgctgttcgagaggaacaagcgactctgacggtggttgaaatggactggatacggcaatactggatccaaggcgtcaagcatgatgagctgcacaccgactggcaaggcccaatgcggaaactcgaagcaaattggctcgtgcttgaggaacgcaccaagactttggtggatgcactactcaaaacacaagacatcgagcatctcaaggtcctcaaagctgttttagaaggtttgcgggctcgtcaggccgggcgagatcaatttgttcatcgcatgagggataaggtgttcaagcgcatccacgccgacttccagccgacagaacgacccacgtggaagtctggcattctgccaaaggacctaacagatgtcattgccaagctgaatgcacacaaggaagagctcgagaaggagaagaagatgcagtggaaagccaaccataagaaacaacaaaagaagtaa

SEQ ID NO: 39 - protein, 689 aa, Ostreococcus tauri
MRRDASGAKEYTGAYATPMKKFYALEPTAVTDFFNNAFGVMLMWACMQAFYVAAKGSAGLKNRFVVANIIAAFSRATVLACHGSKDRASGGMVASEHKLGSFIAVLTMFLLAEAVRVDSNLVPLRHWIKDSKSLKQKIITIALLMEFYYMIILHFGDVTVWFSPKVKATDYLAVHWTATIGFMKWLQTMIMFTAASNSDVQTVAKFDALSTLTMLYVMRKHQAVFKAAEFSKHFTMTVAPLISLTVINNISPHLVWHQSRARPHCASPKMPDVLGPGDGDAYERVRSFHYDGFVVDDPEILPEALHDDVARAFDSLLREGEFTHDVLSAGNKVSRTFVTRTLLGDRGTTYHYQKLRLFAQPWDGEKRWEYATARRLNEALTRRTREILRGTRHGESECEYNVTLINRMDTREESEVALKDEALFDLGTTSVSWHSDSSLRENSTVAVYSTVDDPERADWSVALRALGKQCPALRVPLKDKATYYMCGDFNATHHHAVLTGSSARYSSTHRVGVVEKDTFQYIKRRCLEALALIPDLENKSASLDAKKIQLLAEIHREVEFQWIRMFHLQGDTHARQHKTYWTQRIAELTEAWDRMEACFRAILSKLKLSPESTPQPSRAYSMMLYAMKEVKELRDEHVKRISASAYAALPESQRPVDLPQYDDTSPLPFELKPVIYFVEAEQAKLKGES

SEQ ID NO: 40 - protein, 555 aa, Micromonas pusilla
TSRSRTPRPGMSKAHKRPRDGAARPAGARDSASKKARRKAKSAGGGARDEKRAPGDGSRPVEPPSAKIPSRPPPSYRLSTCDTDEASAASVLRPGDDEYEPCVRRAYAGFHVDPPESLPRETHDAAAGAFESMRAAGLFHRDVLAAGNVVSPTFVRRVLVGDRGMTYHYQKLRIFAHSWHDDDLKTRETTNESRDSSHLSPLRVVRELNETLKKRSARVLRQTRSPPNASSKSDGSCDFNVTLINLMDAKAAALKSDVPLKDESRYGMGPTSVSWHSDSSLQPHSTVAVYHVLFDETDSKRTNSCDDQSWHVALRTLDGRTPALRVPLPSESTYYMLRDFNAYHHHAVIAGNTRRYSSTHRVAVVEKDTFDYIKRVCVDAVRVLPELKTMAESARRKGRGEGAKNVDFSRLARLTQTVGDAHRETEFQWLRMFWLQGEEHAQAHSHYWRGKIEELTQAWDAMELGLRWVLECLRFCADASTPSLTPPTRNYDLALYVLTAVAEAREAYFKRASSAAYAKLPETQKPVRRPAFTETSPLPENLRPVVAALGSDRKS

SEQ ID NO: 41 - protein, 169 aa, Micromonas polaris
STKRYSSTHRVAVVAKDTFEYIKKRAVSAVGKSETLARYARDAKAGRNSTTSKRDSPTDENSLAKLTQFVGEVHRELEFQWIRMFWLQGQDHADRHAEYWNVRIGELTQAWDVCEFALRLILDTLRFASVADGAQSTAPTPPQRTYDLAVYVLQSISQARESHVERASS

SEQ ID NO: 42 - protein, 787 aa, Ectocarpus sp. CCAP 1310/34
MTMTTATTVATTSRLLSPPTSEATTPIQHSNSGMEVSTEKTSHKKKRKSKRKRQQQERDTSSIPDASRQSRGSSSSSTRPPPPPPPPPPTTTTTTTSKKHSRIKEKPARFVHPHLDAELRRLGRSWLSPGDPLFKPALDRSYRGVRHEPGGSLPQAVHRGFRQSFDSLHEAGLFLYDTVQAGGKRLSRTFVTRTLVGDAGITYKYLGLRLFAHPWGLGESDFRKGPGDSLADALNDPCLADLFSNDGSGFDDNDRVADTLRKVGDLSEWMRLRAGAMLKEEVGEAGGERGSCAFNLTLINRMECSGAKRDLKPDPLFSMGKFSVSWHADSCLQDFSTIGVYHCTDPVPAEPDWGIALRVSTEARVGGGGGGSGDGDGYGVSNVEKTRRKEAERGVKPAGPKAIASVAASAAGAVPEKQGEQVLTPPLLIPLQSGDAYFLLDDFNHFHEHAVVSGSSTLRYSSTHRVADTDGNTFSHVQKRCERALAGGGSAAAGGDYHILNLAQVRAEQALLTELESEWLNQFWVQGRRHASLHPWWRGPMALVLATWRELERRTALTVRRLMSLRGQGGGEGVGPGGGGTAAETAGAEPAAIAKAFETVLRALEVRRESRSRWGARFADSAYRSLAADLAPVVPALWPAEGSSGGDVGEEGSPLLLPQSLGGIVSRLRECYTVVYRAASGGKKAGSAKAIAAGAASNWELFRAASSSSSSGGGGAGKKHGSALGKQQQQQQQQQQQQRKMRGLAGQDGLEKTKKRAGGVVKGGGDGGGGRTLKGQGSRRKDRVRER

SEQ ID NO: 43 - protein, 1103 aa, Ectocarpus sp. CCAP 1310/34
MAKSNGDRGRGGRGARGRGRLSKGKGGRGSGSSSQQRHQQTPQHHPARISVKPPSFSGFRGPPDRFVEESHPQFQEIVSKCYAGFHVEAPEAFDAKVHEEARQAFDGLEKGGVFRADVTQPFGLGTKCAMTYVRRTLVGDPGTTYKYLGLRMFSHPWSTTAKEADPDRGHGSGVPSALRRVGLLNDKLKERSLRLLRARGGGKGKARREGGEGNEFGSCDFNVALINRMEPSDDRPDLKLEPTFGQDRCSVSWHADSCLEHYSSIAVYHVTDPEAKNKNANANGGDANPAVPADWRIALRVEPDAEGPSAGKLKLAATENESSRVDRAPAVAVALPSRSAYFLLDDFNHHHQHAVLAGQSHRWSSTHRVAKKQGHSFDYISGRCRTVLRDGHRRTLKQWKTEQLTLAELEFEWIRQFYVQGRAHHALHSWWHGPLQELLRFWSLLEAKTAVVVNTLQGAAESRFDHHHQQQQSGRGVVEAAGAGADDKREKKARQKRAKQRQAVESLGGRAAYSAIAEALGDRAEKRRQWAEREKDKVFERLSADSRPMPCPLSPSFSSANRALSTPLPRTPEALSALADAVLEWGKIFSGEDGVRGGGIVAAEEAGVGEGGRSVSGDCGENESGGTGEKPAGVADSGVADSGAGVHNDPDGQEPCESRTSGGSLPSPVPTSPAGAESARHAPPTPWETGRFALEVQAPWSRRLLDGEKTVETRGYPLPAGLVGRSIEVLESGRGLDGVSGVGDVVEAFSAGLSVVGRVVFGGTEAYPSREAWAADADRHLVPVPAEEGGSESGKGYGWKGPGSVHAWKVASVEAYPKPRAVRRMGRVFRSLFLVEQPNGKQSAGGVGGGGGGNDNGAKPEGSAGAGAADDRPEIGGDEGKRKRKPKKKRKRKGPSAGGVLGAADAAPGTAVSETGKLFSATSAAFETPNATAATSATAVSPPREAKGGSAEKAKTKRPRYAANGQQETVSGEGNVFDGAASGGEESLSNGSESVQDDGGVGGGGGDGGSGVEGSRSGRGGKGKRRRSGNGSEQQKGLTRKGGEEDMGDEGVVALAAAAAAARSAGGGFDVVVGDGWGAADAPAVKPGDGEMRNRKKRKKKRY

SEQ ID NO: 44 - protein, 502 aa, Thalassiosira oceanica
MGKSKKRKLSAEDAAAVSISSKLKKLRGVAKDTKSPPAFHCGKTCASLSPSESDGSLYEECLSTSYSGFRHETVSSALSCQFDTSLEGLDSLYQYDVVQPGKFRMTKTSVTRTLVGNPGSTYKYLGLRLFSHPWSDVDERGERQTLRDLGYSEDIASALLTIGRINTELVDRTNTLLKRHVEPLQPVILPANFSLTLINKMEPSSTKKDLKRETLLGGRKISVGWHRDSGLKDFSSICVYQTIKGQELSSSHDWGVALRTTDHGGPTPPLVVPLPSGSLYYMCSQEAVAVVIRLLTELHVKGKVRRANGTTSPDVRESKFRMQAPGSILETKLHPTSWIDKSMYNPELSRSQQSSSNEVMAILEEASESSSTNEGDRRVGAGCVLVTEDLFDLMIEIYSKREETYAIWQERYSDPIFKELPPNQRPRDCPVLDKEDAPRRLSDLVRQLRSRFVGSVVHDEQNGNTKSEKAKIRKKEKRSSGKYTKKEAKGRASNWLKLKADL

SEQ ID NO: 45 - protein, 869 aa, Fragilariopsis cylindrus
MGKSRSKRKSKDKLSEENISQPQQLPSTTMTASLSLTNGDNNNSVHLPQAMPQHLPRPFGDNFLKDESPYRKSFREALTTSYEGFVFDDAATLMSQPTAINNVKEDVVQNSLESMSRGGIFRTDVTQPFGLGTKCAKTYVTRCLVGSPGTTYKYLGLRMFAHPWTTSTTTGEDNNNNSNMKRNDNERRVCHTVVTNDAQTIQELALALTNRTKKHLRDLDESRRQRQPMFGTRGRPGFDICLINRMESSSDLKPYNFSGDSSTSSSNGKSGNKNGVKTTVSWHADSSLEHFSSIAVYQTILGSQKDESNNNSNDKRKRQRTDCQKQKADEEEEGQWLVALRVAHHSEGPQASQQRRRGTNTETATVEETPPIAVNLPSGSCYYLLDDFNHHHQHTVLTTGNTSTDWIDRGKSAIRQFHKKGSRIWRSEQLLLTEIESEWIRQFYIQGMGHHQLLWESYWKDPIQELLSIWSRLEHRTEQTIELLRAAAEGKCGVGMNTEKAADKPTKAERKARDRRKKSLASIRELVSRINETPEEDGATAFTELYQPMAELLEERAEMRSKWEKREKDHVFHELPLDYRPMKVPFKFERTIDENNIGNEYVATSPLPNSPDKLKEIAAQLLQLGRAYRNGDAKQLPPPWKKEKPKQDALAEGDSTVDDHSKPLDWSGWNACAQLFGLELQHPWAAAIIDGEKVIETRSYSLPPSLIGGTKVMIIESSSGKAGVSSLCNHVDFSTSSRKKGTGNSKVIGWCTFISVKTYTTKQEFQAEENLHLVTPDSGYGWKDDGSTEKVYGWIVGERYRFDESSTTDEENFLYDSGVRRFRSLFQLHKKKSKDAYPNTSNKKNINKRNLERENKQNSNGKNKKRGRY

SEQ ID NO: 46 - protein, 416 aa, Fragilariopsis cylindrus
LTPEDGVYFQKHLQDHYKGFVHLPVDRLEPRDFHEEFKNSLERLRDAGYYQYDVVMAGGKYSSRTFVKRTLVGNPGITYKYLGLRLFAHAWSGPGCTPLMKSIGDMNQSMIKMTEQFPENERCDYNLTLINYMEPTTHTKVGFKDEANYGMGKVSVSWHADSSLVDNSSIGVYHCLPTQRNSKWDWKIALRPLAIESKDNTGSTKRDNKSTTPKPVVVNTKDGDAYFLLGNFNMKHQHCVLAGSQANRISSTHRVAVTIEDTYDYILKRVKIARKRFRLQMETIPPSLQSPVVGLDAKVIRYCQRILTEVEMEWIAQYWLQGDQHDKMRVWWQKPMKTLEAYWCALEVYTYRLFEFLLSRIASNDTVPIDVVKVLIIEFKTRQSFREQWDERRSDKIYQRRVSEEFRPVARPIFEN

SEQ ID NO: 47 - protein, 535 aa, Fragilaria crotonensis
MGKNKTKRGQQDDVATRSHVKSLHSKHSPTFPLPFPAALKYPKNEFLRDEPPYEESFQQALETSYEGFCVDDPMVMKDFHQRVEAALLAMEGAGFFRVDVTQPFGLGTKCAKTYVTRCLVGEPGTTYNEALQTIGDLNAMLTQRTKKHLSDLDQKRIARGAEPTKGRLGFDVALINRMESTVGLKDEPYLGQGKCSVSWHADSSLENFSCIAVYHLINKEADTEGKWSVGLRVAHNSEGPQASRRGTDIYVETETPPLAVSLPSGATYCMLDDFNHHHQHTVIAEGKVPGRRFSSTHRLLRDSHNVAHIIARCKSACANFHKKGPKVWRSEQLLLTEMESEWLRQFFIQGQAHHDLLWKNNWQEPMEELLKYWSRLEERTKQTIEWLQFGAEGRCGMDDSSSTADGPAPPRAERKLREKRRKAREGIEELLGRGEVNSGEPKKYLYEPIALLLQERAVMRELWLAREHDGVFHDVSRDNRPIPLPVDFLVGGSKNSIERGQSPMPGSSQELQDMAARLLCWGKAYESGSRSDFAG

SEQ ID NO: 48 - protein, 338 aa, Fragilaria crotonensis
MGKLNRILVQKTKEQLQSLSEKGKVLGSCEFTLTLVNRMEPSSQKKDLKRDPILQNKTSVSWHKDSGLMDFSSIAVYHKLIGDDVARTKPWRVALRVASPSSKTPALEVPLPSGALYYLLDDFNHQHEHAVLSGATQLRYSSTHRVARGQTWESVKVRCQSILESNQPVASAWQRAKQVRAKQQLWTELEFEWIRQWCIQGARHAELHPYWHGPLKYLEECWKSLNTMTLTVLNDLKNGSPEEDLYDVMIESYQERIALREKWEVRLRDPLYATMDAEAKPMTCESCPTTLAELNSTLANLRTWKKRKFAGGKTKKEKRKVASNWEAMKKSEKKHKHS

SEQ ID NO: 49 - protein, 506 aa, Polarella glacialis
MDMDRFVREPADASSESFHEEFRRCLHGLYEQGIFQYDLIQPVGPGTPVVSTRVTRCLIGEPGITYKYLGLRMFAHPWKGEKTSESCSAMYQHNQELISKAEEQAVAVGCDAPPYSSCFNLTLINRMDPADSGAPNKLGAVSVSWHADSSLVDYSTITVYVAEHDATQDSFGLSPARPCKPCDDPWRVALRHTPNVQGPEKSCPWEVAAQETAKPVKRRAKAKVHTVPIHDGDIYHLIGDFNHHNQHAVLAGRDAVLAVRRGMPVLGGSGTIRYSSTHRVALTEGHSYRSVKQRCSTALSVALADMIPAAAQVHEEQQCSLELEFEWIRQFFIQGKAHREALPWWHSKMDELVFLWRQFEVLTAQRLTLLRRAAAGKVGTSELDPFCLALKNGLQKRAELREAWRQREHDPLLKKQPANRQPLQCLFDAPSDAPHGAGSEALPHNVWPLVEEISQIQLQLHETKKVGKPWYQFVLNVYQGCAFQGGEVTCLSWTLFSQRLPMKRRW

SEQ ID NO: 50 - protein, 918 aa, Polarella glacialis
MSLSLQLVPLQAGLGGPWLLPPVGSAPLTIGRKTSCELLMPEAHAYVSGEHCRVRSVVQESKFCVDLEDLSGNGTYVNGGKVGKGNHRNVCTGDEISLAKPSRRGGAIMFRLEVVSAAVSAAVPAAVPAAPPVAHPVVPAVSATKQATPLEGRSASGDLAAALAVSARQRQGENHRLQALFEAEKVRCASLQAELLEERRQLAEERRRRQESQAAKVLVHAQAAASRAGSSDALLKDNARKQGDCEELRESLRRISAEHAPLERAQLSAEEAAKRESRECVRLRAELQEELRCAERAALEKDQLKRQAEEAIGRQLELEAEVRREAAFNASLEEQCAAMCKAELSLAPSSRMGQVSGCNCFDQEALRAQPPKFRLVSKWCQFHRPKFRRSDQQQQDTMVMQLMPLWNKKHRLYESLGFPSGHLLSAAMDMDRFVREPADASSESFHEEFRRCLHGLYEQGIFQYDLIQPVGPGTPVVSTRVTRCLIGEPGITYKYLGLRMFAHPWKGEKTSESCSAMYQHNQELISKAEEQAVAVGCDAPPYSSCFNLTLINRMDPADSGAPNKLGAVSVSWHADSSLVDYSTITVYVAEHDATQDSFGLSPARPCKPCDDPWRVALRHTPNVQGPEKSCPWEVAAQETAKPVKRRAKAKVHTVPIHDGDIYHLIGDFNHHNQHAVLAGRDAVLAVRRGMPVLGGSGTIRYSSTHRVALTEGHSYRSVKQRCSTALSVALADMIPAAAQVHEEQQCSLELEFEWIRQFFIQGKAHREALPWWHSKMDELVFLWRQFEVLTAQRLTLLRRAAAGKVGTSELDPFCLALKNGLQKRAELREAWRQREHDPLLKKQPANRQPLQCLFDAPSDAPHGAGSEALPHNVWPLVEEISQIQLQLHETKKVGKPWYQFVLNVYQGCAFQGGEVTLV

SEQ ID NO: 51 - protein, 879 aa, Pseudo-nitzschia multistriata
MGKMGKSGSKRKQQRAAAATTTTVSLDQTVQTTKPNREQGGDIVLLPQEFPPHLPKPPRGSFLRDEAPYRDSFGEALSGPYEGFAVDDAKELSLPCEGDNTGIHEEIVLSSLETMEGEGIFRTDVTQPFGLGTKCAKTYVTRCLVGERGTTYKYLGLRMFAHPWTDGSEEAADDRGGKKRKRRKRAQGCDAKVDQLNQAFDEAKTTSTIQELGKSLTMRTANHLRDLDQRRRQRRAPATRGRPNFDICLINRMESSPDLKPYNFGVGEHNNKSSRSGKNEGNGTKTSVSWHADSSLEHYSTIAVYQTLLGGCRTGNDKSKSKATKKNSDEGKWWVALRVAHHSEGPQASQRRRGSTTEASIVEDTPPIAVSLPSGSAYYLLDDFNHHHQHTVLTTGDAATAGVRYSCTFRLLRDSHNVRDWIDRGNAAIRQFHKKGPKLWRSEQLLLTEIESEWIRQFYIQGTGHHKLLWEPYWKNPMKELLSIWSQLEARTKQTMDLLRAAAEGKCGLDAGNGTETKPTKAERKARDRRRKALGTIRDLVSRIHIHNDDETVTELYLPFAELLEARAAMRELWGKREKDHVFHEMPPDCRPMEVPLRFGDQTAAHRSSGAAERDGSTGNSPLPASPAELRELAKQLARCGKAYREGNAALLPPPWDTDERKPPSPTPSIEKKDGSPRDHHAKPMDWPGWDADGTTFGLELQQPWAGFVANGKKAIDVRSYALPPALLGKKVMIIESASGRAGVSALGNRLRLTGGGTTRVIGWCVFSSVKAYETGAAFRADERLHLVAPDNPYGWKDGTTKNAFGWVVGEHHRFGESSPAPSGAAAMGSGIRRFRSLFQLAAEEGVEPNPAREKKNNSKRSSATRKKKPNGRKKRKRF

SEQ ID NO: 52 - protein, 867 aa, Nitzschia inconspicua
MPKSRHRQRKNVDGTHNQAAAPPVPSSEMSSIIHLPPPFPSNLSKPSNGFLRDESPYRESFQAAIDGPYEGFVIDSVQELQQEKPTSDSLILQTDPVQDRYSRQTNNESEFDEQKSSGKNDQKKHKITESFVSFALNAMNDRGEFRQDVTQPFGLGTKCAKTYVTRCLVGEPGTSYKYLGLRMFAAPFECNDSTKAIRQLGALMSQRSAIHMHKLNERRRACGAPPCKGMNPSFNICLINRMVYTTDLKPTMALPSDSDDRHAKTCVSWHADSSLEHYSTIAVYQTLETPNSKKVKNKTNKESSLSKDWWVGLRVVHHAEGPNASSGRKGSSIEAALVPETPPVAVSLPSGSAYYLLDDFNHHHHQHVVFTTGNDDSTGVRYSCTFRKLRESHNVQYWIERATALKKSFNKKGAKIWKAENLVSQELEFEWIRQFYIQGKRHYDCLWAAHWKKPLQELLSLWSCLEHRAYQTIELLRAAAEAKCGMDLDNSACRPSKGERKERDRRRKALSTVEALLSRSDAEVGSLGEGASSQLYIPMAECLEEQAKKRELWEKRERDHVFREIHPDCRPLPVPFRFHRPVGEPVLPSCEWGSSPFPTAPTALKELARQLRQLGEAFDEGDTSKIALPSWKHAVSMEWGGWEEGTFGLELQEPWAGAVVDGSKSIETRLYPLPPALLGKRIYVLESKSGAAGVSGLGNFIDIGNHQSETADGESAVDSGRHVHIIGWCTISSVKKYNHRIEFEQDESRHLVTSDSGYGWTDNGTPIYGWVLGDYHRYEYEHRRNDAQQPSFSSGVRRFRSLFELSQNSRPVDVVDTNTNNNYSESFDASTSVSGKKKGKRNNKGHRHNHNHSNSNNAGKVKRRKRF

SEQ ID NO: 53 - protein, 106 aa, Ostreococcus mediterraneus
VSSTFVRRVLLGKRGATYHYQRLRLFAVPWEDEHTDRDSPHRVMRRLNEALIERSAKVLSGTRHAESKHEYNVTLINYMDTERASEVELKCETLYGLGTTSVSWHS

SEQ ID NO: 54 - protein, 211 aa, Sphaeroforma artica JP610
MTYHYQRLRIFALPWDDDGTGVYQADTAKCESGRPVSNLLALKQLNSTLTTNATELLLQYHKEGGDKHNQQIAQKGVVPRGSCKYSVSLINYMFCEKDSKVPLKNEAVYGMGPTSVSWHADSSLQNYSTIAVYQITGNGPIAGSQNQTKRSSKTSDTTKGSEDDWSVAMRVVADDVTPAVACRLKSGETYYMMDDFNHHHHHAGMQHSNGT

SEQ ID NO: 55 - protein, 540 aa, Schizochytrium aggregatum
AEAAAAGGRSDPKPRAMRSRWARGSFLKDGDALMEEALRVSYPGFAREPLQGKGLHEEMRDALHALLQEGYFHYDCVQYGARVNVTRVQRVLVGEPGITYLYQKLRLFALPWDDASVARGALVSIAKLNEQVKRQARKMLAQKGAEGGHVAASAGVGSCDFNLTLINAALTNKHAPPEEGVAGNRKTKKLKGATAADEESENRGPPRQPVLPFKPDTKFDAPIDLAVNWHRDSTLEPFSTIGVYHHTPDSSARDWEIAMCCDAGDLAPPLRTKLSDGECYFMLGNFNHHHQHAVIAGTSARYASTHRVALTQDATFDGIMERCAEVLCGKRFTRSASPESLEAARARAVGELAARLEFDWIRQFYCQGAVHAAQHSTWWAPRIDQLTEKWTELDTELRTMAEVASQQQVPLKALRMLIYLFESRLKGREEWEARLRDPTYKRIPAAYRPIRRPDCSTEGAFTAGMDGLLAALQARLAVRTNSKAAKVAKDTNVTKAAKATKATDITNSAKAAKGAKVAHATTAAKASRVGKAMKARANRS

SEQ ID NO: 56 - protein, 167 aa, archaeon
MTYKYLGLRMFAFPWTPQSLGTNSDLIAINQLNQSLIHRTSTLLNELNRESVGSCMYNITLINRCYSAVDVKLKKEPMFDKDLCTVSWHADSGLEHYSSIAVYHFEKTPLPSPPSPWKIGLRVLINAEGPHQNKPPLYSTPQSLPPKSTPTPSAAHTPASALIPPVC

SEQ ID NO: 57 - protein, 642 aa, Seminavis robusta
MTKKSSNARRRARKKAQQEAAQQPGTSKGKKRQLQEKIHSSSDPHPTSANKKKRKRNQDNLNHHKNKKNVKEDDDDLLAFAQAWASNTNNNNNNQRNEKEKNPVVNDVDRPLPPFPSRLPVPTDGFLRDEDPYRESFQAAVDTVHQGLVWETSLTSTTTSLSEDQIQRALVTMDDAKLFRTDVTQPAGLGAKLVPSYVTRCLLGDEGTTYRYLGLRMFAHPWNGKHTSTDQKLQSAVTTFGSLNQHLSRQTKKHLTAFHEKRGIDQSSKQDNNKKACFTVTLINKMTPHKRLREEPMFQNNKYSVGWHADSSLEHFSTIAVYHTILAAEATTKHSSKKQDDDEWAVALRVTRHAEGPKAKRLGDITVDDTGPPIAVALPSGSTYYMLHDYNHHHQHAVLAPPTHTNKSTRYSSTHRLLRPGHTVQEMIAKCQHTNMQHHGPKKWRSEQLLLNELENEWLRQFFIQGPEHQQLLWGYWKTPLTQLFDYWERLEARTLQVVTLLGNAAVARCHNDNDNNKKQYQEALAIVMQVQGDRKKKNNALETIYEPFAKLLLERAKSRELWAKREKDPVFAKMPKECRPIPFPVAYGKENKKDHDSMAHSSLPDGSPTFLTQLAVQVQAWGRAFDSRNAKHLPNDNVLHC

SEQ ID NO: 58 - protein, 718 aa, Seminavis robusta
MKRHSNDKTKPNKQKRRNQKKVEKKTDTSTSTPVQLNKGQVLPFPRGLSFPKDDFLRHEAPFQESFAAALETCYLGLAIDDPSTLARHIPVPLEQKKGNLDKFPLQETLEEMENRNFFQADITQPFGLGSKCAKTFVTRCLVGDTGTTYKYLGLRMFAYTWNSNSNQKNNDNDKLCQEIKRFKDDILVPRTKQHLKSLVHKRKQANANGHSNANDTDISRSRPDYNICLINRMDASNPQYKLEPSLQHDKIAVAWHADSILEHYSNIAVYQLITNNQNNQHIHKDSNGRQDWSLALRVVPNAEGPNIQADKITPDDSTPAVSLSLPSGSAYYMLDDFNHHHQHAVLRNSNQNESENENEIKSKKKARNNLKQQKPPQDSSPSSSSSSSSIRYSCTYRLLRPSHTVQEALEQCRKICAGFHKRGPKIWRSEQLCLTYIESEWLRQFYIQGQHHYDMLWHNNNWGPPLKQLWKYWAQLETRTQQTVQLLQWAAAQAQAQAQAQPNNNNAPTISRKQRDKQRKAFATVQELLARQSGTTTTNTNSNALQLLYEPLAELLRERATLRLQWQKREKDHVFTTVDPRYRPLPWPVQYENDSNSNNNHQVSTEIEIMASSPLPQDLRPVVDQLMAAGRTSYCYSDNQDSRQSSSGLAKPPVATTRDNDHDSMGGKAAGSEKSNRAMPTSGSQGHISKRNKKRNKTKNGKRNNDDASAPKKKKRRF

SEQ ID NO: 59 - protein, 862 aa, Nitzschia inconspicua
MPKSRHRQRKNVDGTHNQAAAPPVPSSEMSSIIHLPPPFPSNLSKPSNGFLRDESPYRESFQAAIDGPYEGFVVDSVQELQQEKPTSDSLILQPDPVQHRYSQQTNAESEFDEQKSSGKNDQKKLKVTEAFVSFALNAMNDRGEFRQDVTQPFGLGTKCATTYVTRCLVGEPGTSYKYLGLRMFAAPFECNDSTRAIRQLGALMSERSAIHMHKLNERRRACGAPPCKGMNPSFNICLINRMVYTTDLKPTMALPSDSDDRQAKTSVSWHADSSLEHYSTIAVYQTLETPNSKNFKNKTNKESSLSKDWWVGLRVVHHAEGPNASSGRKGSSIEAALVLETPPVAVSLPSGSAYYLLDDFNHHHQHVVFTTGNETSTGVRYSCTFRKLRESHNVQYWIERATAFKKSFNKKGAKIWKAENLVSQELEFEWIRQFYIQGKRHYDCLWVAHWKKPLQELLSLWSCLEHRAYQTIELLRAAAEAKCGMDLDNSACRPSKGERKERGRRRKALSTVEALLSRSDAEVGSGASSQFYIPMAECLEEQAKKRELWDKRERDHVFREMHPDCRPLPVPFRFYRPVGEPVLPSCEWGSSPFPKAPTALKELARQLRQLGEAFDEGDASKIVLPSWKHAVSTEWGGWEEGTFGLELQEPWAGAVVDGSKSIETRLYPLPPALLGKRIYVLESKSGAAGVSNLGNFIDIGSHQLETADGESAVDSGSHVDIIGWLTVSSIKKYNHRIEFEQDESKHLVTSDSGYGWKDDGTPIYGWVLGDYHRYDCEHRKNDDAQQPSFSSGVRRFRSLFELSQNSRPVDVVDTNTNNSNSESFDASTGVSGKNKGKRSNKGHHHHHSNSNNAGKVKRRKRF

SEQ ID NO: 60 - protein, 551 aa, Nitzschia inconspicua
MSPMASNERKKKRKRRKSASGSVAESSPAGTFSDHLESTARLSNNSSVILSPSSSQIPPVLSSRSFLTSNDNGFTNYVKDHYPGFVHLHVVQYEQTLQQHDFHAYARQALERLRDASYYQYDIVMAGGKHSSRTFVKRTLVGEPGITYKYLGVRLFAHAWSGPLASPAMKAIGEVNRRMIEITKQMPNPGSCNYNLTLINYMEPTSHTSIGFKEEANYGMGKVSVSWHADSSLESGSSIGVYHCFPTQRAARWDWRIALRPSPDSNKPGNIAPVVVNTKDGDLYFLLGDFNENYQHCVLAGSQANRISSTHRVAVTAEDTYDYILKRVKIARKRFRLQLEGNEDDKTFKSPSPTTLDAKIIRYCQRVLTELEMEWIAQYWLQGAEHDKMHVWWQRPMKTLEAYFSALEEYTYKLCQLCVNIDTSDMVPVDVLKVLLAEFKSRQSLRQHWDERRGDKIYQRRISAEYRPVARPSFLSKEQQDGTKLDERLPKDLSSTIHQLSQVLKKRSGNVHTNEQNGSLSTGCLTTGTKTNTSTGSKERIEKKKKKQRVH

SEQ ID NO: 61 - protein, 376 aa, Aphanomyces astaci
MSPHMNLGITYKYLGLRIFAHAWDVPSSAAVFRNLYALNQAIVARTKASASPAPPSCEYNLTLINYMEPDNPVLGLKDDLTYDMGKVSVSWHADSSLEDFSSIGVYHTLLPTKKSKKGPCDWRIAMRLSPDVPGAKTTPPLVVPTGDGDVYYLNGDFNHHHQHMVLSGTAVRVSSTHRVAVTAEDTLQYIQAKVTAALKSSAKDPQDKCRRGHADDVRHEQTTVTTLEMDWLRQYWIQGDKHNDLHVDWQQPMLALEASWRKLEARTKTLADALVQSHDVDDLKVLKAVLEGLRNRQVGRDQFVHRMKDKVFKRIAADFQPMERPVWTDWDDVHLLPKDLTATIAALHAHKLVLESEKKRQWKIHAGTRHNKINKA

SEQ ID NO: 62 - protein, 522 aa, Aphanomyces astaci
MDPKERKKLERQKKKAKALERKNAKKPNVASLKPVSQPTTSHKRPSEPSGSSAAPPAKKQQIEKAKPLPVPSNYTGPEKFLTPSHPDFHDILTRSYVGFVHEPAAAQPREFHAGMRAAFEHLRDKGYFQYDVVMAGGKHLSRTFVRRTLVGNFGITYKYLGLRIFAHAWDIPSSAAVFRNLYALNQAIVARTKASASPAPPSCEYNLTLINYMEPDNPVLGLKDDPTYDMGKVSVSWHADSSLEDFSSIGVYHTLLPTKKGKKGPCDWRIAMRLSPDVPGAKTTPPLVVPTGDGDVYYLNGDFNHHHQHMVLSGTAVRVSSTHRVAVTAEDTLQYIQAKVTAALKSSAKDPQDKCRRGHADDVRHEQTTVTTLEMDWLRQYWIQGDKHNDLHVDWQQPMLALEASWRKLEARTKTLADALVQSHDVDDLKVLKAVLEGLRNRQVGRDQFIHRMKDKVFKRIAADFQPMERPVWTDWDDVHLLPKDLTATIAALHAHKLVLESEKKRQWKIHAGTRHHKNNKA

SEQ ID NO: 63 - protein, 522 aa, Aphanomyces astaci
MDPKERKKLERQKKKAKALERKSAKKPNVASLKPVSHPTTSHKRPSEPSGSSAAPPAKKQQIEKAKPLPVPSNYTGPEKFLTPSHPDFHDILTRSYVGFVHEPAAAQPREFHAGMRAAFEHLRDKGYFQYDVVMAGGKHLSRTFVRRTLVGNFGITYKYLGLRIFAHAWDIPSSAAVFRNLYALNQAIVARTKASASPAPPSCEYNLTLINYMEPDNPVLGLKDDPTYDMGKVSVSWHADSSLEDFSSIGVYHTLLPTKKSKKGPCDWRIAMRLSPDVPGAKTTPPLVVPTGDGDVYYLNGDFNHHHQHMVLSGTAVRVSSTHRVAVTAEDTLQYIQAKVTASLKSSAKDPQDKCRRGHADDVRHEQTTVTTLEMDWLRQYWIQGDKHNDLHVDWQQPMLALEASWRKLEARTKTLADALVQSHDVDDLKVLKAVLEGLRNRQVGRDQFVHRMKDKVFKRIAADFQPMERPVWTDWDDVHLLPKDLTATIAALHAHKLVLESEKKRQWKIHAGTRHHKINKA

SEQ ID NO: 64 - protein, 522 aa, Aphanomyces astaci
MDPKERKKLERQKKKAKALERKSAKKPNVASLKPVSHPTTSHKRPSEPSGSSAAPPAKKQQIEKAKPLPVPSNYTGPEKFLTPSHPDFHDILTRSYVGFVHEPAAAQPREFHAGMRAAFEHLRDKGYFQYDVVMAGGKHLSRTFVRRTLVGNFGITYKYLGLRIFAHAWDIPSSAAVFRNLYALNQAIVARTKASASPAPPSCEYNLTLINYMEPDNPVLGLKDDPTYDMGKVSVSWHADSSLEDFSSIGVYHTLLPTKKSKKGPCDWRIAMRLSPDVPGAKTTPPLVVPTGDGDVYYLNGDFNHHHQHMVLSGTAVRVSSTHRVAVTAEDTLQYIQAKVTASLKSSAKDPEDKCRRGHADDVRHEQTTVTTLEMDWLRQYWIQGDKHNDLHVDWQQPMLALEASWRKLEARTKTLADALVQSHDVDDLKVLKAVLEGLRNRQVGRDQFVHRMKDKVFKRIAADFQPMERPVWTDWDDVHLLPKDLTATIAALHAHKLVLESEKKRQWKIHAGTRHHKINKA

SEQ ID NO: 65 - protein, 522 aa, Aphanomyces astaci
MDPKERKKLERQKKKAKALERKSAKKPNVASLKPVSQPTTSHKRPSEPSGSSAAPPAKKQQIEKAKHLPVPSNYTGPEKFLTPSHPDFHDILTRSYVGFVHEPAAAQPREFHAGMRAAFEHLRDKGYFQYDVVMAGGKHLSRTFVRRTLVGNFGITYKYLGLRIFAHAWDVPSSAAVFRNLYALNQAIVARTKASASPAPPSCEYNLTLINYMEPDNPVLGLKDDLTYDMGKVSVSWHADSSLEDFSSIGVYHTLLPTKKSKKGPCDWRIAMRLSPDVPGAKTTPPLVVPTGDGDVYYLNGDFNHHHQHMVLSGTAVRVSSTHRVAVTAEDTLQYIQAKVTAALKSSAKDPQDKCRRGHADDVRHEQTTVTTLEMDWLRQYWIQGDKHNDLHVDWQQPMLALEASWRKLEARTKTLADALVQSHDVDDLKVLKAVLEGLRNRQVGRDQFVHRMKDKVFKRIAADFQPMERPVWTDWDDVHLLPKDLTATIAALHAHKLVLESEKKRQWKIHAGTRHNKINKA

SEQ ID NO: 66 - protein, 399 aa, Aphanomyces invadans
MDPKERKKLERQKKKAKAQQRKSSKLSNAIPPMPVAKQATNAGNSTSMSQKRPSENAPQCAAAPPTKKQQVKQAKALPIASNYTGPEKFLTPSHPSFHDILTRSYVGFVHEPAAKHPRGFHTGMRTAFETLRDKGYFQYDVVMAGGKHLSRTFVRRTLVGNYGITYKYLGLRIFAHAWDRPNSAPVFRNIHALNAAFIARTKASMTPPPPSCEYNLTLINYMEPDNPLLGLKEDPTYGMGKVSVSWHADSSLEDYSSIGVYHTLLPSKPAKKGTKVGVCDWRIAMRLSPDVPGAKTTPPLVLPTGDGDVYYLNGDFNHFHQHMVLSGTAVRVSSTHRVAVTAEDTLQFIQAKVSAALQSSTVQSVPNDADVACRRGHADAVRAEQAVRPSLVHVVLEDV

SEQ ID NO: 67 - protein, 512 aa, Aphanomyces euteiches
MDAKERKKLERLKKKLKAQKKKEASAPKPASTAPSVPAKPAPKSLKRPAPEHPTAPATKKQDTTTKAFPVPSPYSGPEKFLTPSHPQFNEILKRSYVGFVHEPGTTLPRDFHDGMRSAFEKLRDNEYFQYDVIMAGGKHLSRTFVRRTLVGDYGITYKYLGLRIFAHAWDKPHCAPVFKSLYQLNQRMIERTKRSMKPTPPLCDYNLTLINYMEPDNPMLGLKADPTYDMGKVSVSWHADSSLQDYSSIGVYHSLLGNKKKKAPCDWRIAMRLSPEVPGAATTPPLVLPTDDGDVYYLNGDFNHFHQHMVLAGTAVRVSSTHRVAVTDEDTLQYIQGRVARALKSSANGNSPFRDGEVDAVREEQATLTVVEMDWIRQYWIQGVKHDELHTDWQGPMRKLEASWLVLEERTKTLVDALLKTQDIEHLKVLKAVLEGLRARQAGRDQFVHRMRDKVFKRIHADFQPTERLTWKSGILPKDLTDVIAKLNAHKEELEKEKKKQWKANHKKQQKK

SEQ ID NO: 68 - protein, 512 aa, Aphanomyces euteiches
MDTKERKKLERLKKKLKAQKKKEASAPKPASTAPSVPAKPAPNSLKRPAPEHPTAPATKKQDTTTKAFPVPSPYSGPEKFLTPSHPQFNEILKRSYVGFVHEPGTTLPRDFHEGMRSAFEKLRDNEYFQYDVVMAGGKHLSRTFVRRTLVGDYGITYKYLGLRIFAHVWDKPHCAPVFKSLYQLNQRMIERTKRSMKPTPPLCDYNLTLINYMEPDNPMLGLKADPTYDMGKVSVSWHADSSLQDYSSIGVYHSLLGNKKKKAPCDWRIAMRLSPEVPGAATTPPLVLPTDGGDVYYLNGDFNHFHQHMVLAGTAVRVSSTHRVAVTDEDTLQYIQGRVARALKSSANGNSPFRDGEVDAVREEQATLTVVEMDWIRQYWIQGVKHDELHTDWQGPMRKLEASWLVLEERTKTLVDALLKTQDIEHLKVLKAVLEGLRARQAGRDQFVHRMRDKVFKRIHADFQPTERPTWKSGILPKDLTDVIAKLNAHKEELEKEKKKQWKANHKKQQKK

SEQ ID NO: 69 - protein, 455 aa, Saccoglossus kowalevskii
KQKLLSAVKETQVKFLSPVDTDYKALVAENYDNFVVQPGTELSSELHTRLQQGFETLLTNGYLYQHRIRFRNKTVMTPVSRTLIGEPGMTYRYLNLRLFALPWTFDDKKKLSDVEKACNAFYELNTYFKDHVNKLITEKFASSTGRKIDETDDSLKTIIQRNTEFKATILNYMDPSEIRLKQEPYYNMGDIAVSWHKDESLVDGSTVAVYNHVPMEARQDCSKWKVGLKVAWDITTPAVCTPLNNKDCYYMLDTMNDTHQHCVIAGTLPRYSSTHRVVESSDSTLCYIQQRCQTALDNIGYKDQIPYLKSLETDVLQLIEEIHSEIEFEWLRQFWMQGTQHSQERQYWLKPMEQLQEDWKTMELMTKLMIDIVVKKKDQSHMIAKILLPSLVERNELRNEWMNRYSEKEFYHIPEEHRPIYEPCWTDANSEMPLPFDLSQHIDIIQECMDTNDSQ

SEQ ID NO: 70 - protein, 496 aa, Patiria miniata
MSKMSKKRQNEDQGKELKRKKLLSEVARGKLQFLMPDHPDFKSLEELPWALNILHHHGYFYRDWVKVKGQTVQTPVSRTLIGEPETTYKYLGLRLFAIPWCFGDHQVESEVELACGIIGKLNDHFKAVSRKLLLERAAEKTEPESCISQGAAQSPLHHSPNEETKSCSEQKPTTCSPSIEEGTDFNVVLINYMDPKDPDLRLKAEPYYGMGPLAVSWHMDSNLVQGSTVAVYNHTCSSDGDSSSDSSDWMVGLKVSWDIETPGVVVPLQTGESYFMLGDLNDTHQHCVVAGGQARFSTTHRVADCCQGTLPYIQRRCKEALSNLGETENGEYKLTSLAADHLELTETIHNEVEFEWLRQFWMQGSRHAVERKSWVPAMEHLEADWHKMEHMTKLAVEEANVLDDEEGSAIAEVLLPHLEERQKLRQDCLDKFSPKILETMHADFLPIHRPKWEDDDLSRPLPFDLSNIISNMRLVTKHGKKSCISDSESSDSDYHS

SEQ ID NO: 71 - protein, 329 aa, Patiria miniata
MSKMSKKRQNEDQGKELKRKKLLSEVARGKLQFLMPDHPDFKSLMREKYFNFAHEPSSCIPTSLQEELPWALNTLHHHGYFYRDWVKVKGQTVQTPVSRTLIGEPETTYKYLGLRLFAIPWCFGDHQVESEVEVACGIIGKLNDHFKAVSRKLLLERAGEKTKPESCNSQGAAQSPLHHSPSEETKSCSQXKTTTYSPSIQEGTDFNVVLINYMDPKDPDLRLKAEPYYGMGPLAVSWHMDSNLVRGSTVAVYNHTCSSDGDSSSDSSDWMVGLKVSWDIETPGVAVPLQTGESYFMLGDLNDTHQHCVVAGGQARFSTTHRVADVSFG